(19) United States
(12) Patent Application Publication
(10) Pub. No.: US 2024/0262910 A1
(43) Pub. Date: Aug. 8, 2024

(54) ANTI-CD33 ANTIBODIES AND USES THEREOF

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US); TRI-INSTITUTIONAL THERAPEUTICS DISCOVERY INSTITUTE, INC., New York, NY (US)

(72) Inventors: Anthony Daniyan, Somers, NY (US); Renier J. BRENTJENS, Short Hills, NJ (US); Ivo C. LORENZ, New York, NY (US); Abdul KHAN, Cedar Park, TX (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US); TRI-INSTITUTIONAL THERAPEUTICS DISCOVERY INSTITUTE, INC., New York, NY (US)

(21) Appl. No.: 18/592,427

(22) Filed: Feb. 29, 2024

Related U.S. Application Data
(63) Continuation of application No. PCT/US22/42448, filed on Sep. 2, 2022.
(60) Provisional application No. 63/240,220, filed on Sep. 2, 2021.

(51) Int. Cl. C07K 16/28; A61K 39/00; A61K 47/68; G01N 33/58; G01N 33/68

(52) U.S. Cl. CPC C07K 16/2803; A61K 47/6849; G01N 33/58; G01N 33/6854; A61K 2039/505; C07K 2317/24; C07K 2317/565; C07K 2317/92; G01N 2333/70596

(57) ABSTRACT

The presently disclosed subject matter provides antibodies or antigen-binding fragments thereof that bind to CD33 and methods of using such antibodies or antigen-binding fragments thereof same.

### CROSS REFERENCE TO RELATED APPLICATIONS

The present application is a Continuation Application of International Patent Application No. PCT/US2022/042448, filed on Sep. 2, 2022, which claims priority to U.S. Provisional Patent Application No. 63/240,220, filed on Sep. 2, 2021, the contents of each of which are incorporated by reference in its entireties, and to each of which priority is claimed.

### SEQUENCE LISTING

The present application contains a Sequence Listing which has been electronically submitted and is hereby incorporated by reference in its entirety. Said Sequence Listing, created on Feb. 29, 2024, is named 072734_1557. xml and is 102,313 bytes in size.

### 1. FIELD OF THE INVENTION

The presently disclosed subject matter relates to antibodies that bind to CD33, and methods of using such antibodies.

### 2. BACKGROUND OF THE INVENTION

Acute myeloid leukemia (AML) is the most common and most lethal form of acute leukemia in the United States. For the last four decades, the standard of care has been chemotherapy with the recent addition of a CD33 antibody-drug conjugate (ADC) and targeted small molecules to the armamentarium. Despite these new additions, AML continues to be a devastating disease with a 5-year survival of less than 30%. Hence, there is a critical need for novel AML interventions.

Given the significant role for CD33 in diseases (e.g., AML), antibodies that bind to CD33 and methods of using such agents, are desired.

### 3. SUMMARY OF THE INVENTION

The presently disclosed subject matter provides antibodies or antigen-binding fragments thereof that specifically bind to CD33, and methods of using the antibodies or antigen-binding fragments thereof.

In certain embodiments, the CD33 antibody or an antigen-binding fragment thereof comprises a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, SEQ ID NO: 18, SEQ ID NO: 28, SEQ ID NO: 35, SEQ ID NO: 43, SEQ ID NO: 52, SEQ ID NO: 62, SEQ ID NO: 72, SEQ ID NO: 81, or SEQ ID NO: 88.

In certain embodiments, the anti-CD33 antibody or an antigen-binding fragment thereof comprises a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, SEQ ID NO: 19, SEQ ID NO: 29, SEQ ID NO: 44, SEQ ID NO: 53, SEQ ID NO: 63, SEQ ID NO: 73, SEQ ID NO: 82, SEQ ID NO: 89, or SEQ ID NO: 92.

In certain embodiments, the anti-CD33 antibody or an antigen-binding fragment thereof comprises (a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, SEQ ID NO: 18, SEQ ID NO: 28, SEQ ID NO: 35, SEQ ID NO: 43, SEQ ID NO: 52, SEQ ID NO: 62, SEQ ID NO: 72, SEQ ID NO: 81, or SEQ ID NO: 88; and (b) a light chain variable region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 9, SEQ ID NO: 19, SEQ ID NO: 29, SEQ ID NO: 44, SEQ ID NO: 53, SEQ ID NO: 63, SEQ ID NO: 73, SEQ ID NO: 82, SEQ ID NO: 89, or SEQ ID NO: 92.

In certain embodiments, the heavy chain variable region and the light chain variable region of the anti-CD33 antibody or antigen-binding fragment thereof are selected from the group consisting of:
(a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9;
(b) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 18, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 19;
(c) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 28, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 29;
(d) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 35, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 29;
(e) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 43, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 44;
(f) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 52, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 53;
(g) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 62, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 63;
(h) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 72, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 73;
(i) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 81, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 81;
(j) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 88, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 89; and
(k) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 92.

In certain embodiments, the anti-CD33 antibody or antigen-binding fragment thereof comprises a heavy chain variable region comprising the amino acid sequence set forth in SEQ ID SEQ ID NO: 8, SEQ ID NO: 18, SEQ ID NO: 28, SEQ ID NO: 35, SEQ ID NO: 43, SEQ ID NO: 52, SEQ ID NO: 62, SEQ ID NO: 72, SEQ ID NO: 81, or SEQ ID NO: 88.

In certain embodiments, the anti-CD33 antibody or antigen-binding fragment thereof, comprises a light chain variable region comprising the amino acid sequence set forth in SEQ ID NO: 9, SEQ ID NO: 19, SEQ ID NO: 29, SEQ ID NO: 44, SEQ ID NO: 53, SEQ ID NO: 63, SEQ ID NO:

73, SEQ ID NO: 82, SEQ ID NO: 89, or SEQ ID NO: 92.

In certain embodiments, the CD33 antibody or antigen-binding fragment thereof comprises a heavy chain variable region comprising the amino acid sequence set forth in SEQ ID NO: 8, SEQ ID NO: 18, SEQ ID NO: 28, SEQ ID NO: 35, SEQ ID NO: 43, SEQ ID NO: 52, SEQ ID NO: 62, SEQ ID NO: 72, SEQ ID NO: 81, and SEQ ID NO: 88; and a light chain variable region comprising the amino acid sequence set forth in SEQ ID NO: 9, SEQ ID NO: 19, SEQ ID NO: 29, SEQ ID NO: 44, SEQ ID NO: 53, SEQ ID NO: 63, SEQ ID NO: 73, SEQ ID NO: 82, SEQ ID NO: 89, or SEQ ID NO: 92.

In certain embodiments, the anti-CD33 antibody or antigen-binding fragment thereof comprises a heavy chain variable region and a light chain variable region, wherein the heavy chain variable region and the light chain variable region are selected from the group consisting of:
(a) a heavy chain variable region comprising the amino acid sequence set forth in SEQ ID NO: 8, and a light chain variable region comprising the amino acid sequence set forth in SEQ ID NO: 9;
(b) a heavy chain variable region comprising the amino acid sequence set forth in SEQ ID NO: 18, and a light chain variable region comprising the amino acid sequence set forth in SEQ ID NO: 19;
(c) a heavy chain variable region comprising the amino acid sequence set forth in SEQ ID NO: 28, and a light chain variable region comprising the amino acid sequence set forth in SEQ ID NO: 29;
(d) a heavy chain variable region comprising the amino acid sequence set forth in SEQ ID NO: 35, and a light chain variable region comprising the amino acid sequence set forth in SEQ ID NO: 29;
(e) a heavy chain variable region comprising the amino acid sequence set forth in SEQ ID NO: 43, and a light chain variable region comprising the amino acid sequence set forth in SEQ ID NO: 44;
(f) a heavy chain variable region comprising the amino acid sequence set forth in SEQ ID NO: 52, and a light chain variable region comprising the amino acid sequence set forth in SEQ ID NO: 53;
(g) a heavy chain variable region comprising the amino acid sequence set forth in SEQ ID NO: 62, and a light chain variable region comprising the amino acid sequence set forth in SEQ ID NO: 63;
(h) a heavy chain variable region comprising the amino acid sequence set forth in SEQ ID NO: 72, and a light chain variable region comprising the amino acid sequence set forth in SEQ ID NO: 73;
(i) a heavy chain variable region comprising the amino acid sequence set forth in SEQ ID NO: 81, and a light chain variable region comprising the amino acid sequence set forth in SEQ ID NO: 82;
(j) a heavy chain variable region comprising the amino acid sequence set forth in SEQ ID NO: 88, and a light chain variable region comprising the amino acid sequence set forth in SEQ ID NO: 89; and
(k) a heavy chain variable region comprising the amino acid sequence set forth in SEQ ID NO: 8, and a light chain variable region comprising the amino acid sequence set forth in SEQ ID NO: 92.

In certain embodiments, the anti-CD33 antibody or antigen-binding fragment thereof comprises a heavy chain constant region and/or a light chain constant region. In certain embodiments, the heavy chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 95. In certain embodiments, the heavy chain constant region comprises the amino acid sequence set forth in SEQ ID NO: 95.

In certain embodiments, the light chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 96 or SEQ ID NO: 99. In certain embodiments, the light chain constant region comprises the amino acid sequence set forth in SEQ ID NO: 96 or SEQ ID NO: 99.

In certain embodiments, the anti-CD33 antibody or an antigen-binding fragment thereof comprises (a) a heavy chain constant region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 95; and (b) a light chain constant region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 96 or SEQ ID NO: 99.

In certain embodiments, the anti-CD33 antibody or antigen-binding fragment thereof comprises:
(a) a heavy chain constant region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 95, and a light chain constant region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 96; or
(b) a heavy chain constant region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 95, and a light chain constant region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 99.

In certain embodiments, the antibody or antigen-binding fragment thereof comprises a heavy chain constant region comprising the amino acid sequence set forth in SEQ ID NO: 95.

In certain embodiments, the antibody or antigen-binding fragment thereof, comprises a light chain constant region comprising the amino acid sequence set forth in SEQ ID NO: 96 or SEQ ID NO: 99.

In certain embodiments, the antibody or antigen-binding fragment thereof comprises a heavy chain constant region comprising the amino acid sequence set forth in SEQ ID NO: 95; and a light chain constant region comprising the amino acid sequence set forth in SEQ ID NO: 96 or SEQ ID NO: 99.

In certain embodiments, the antibody or antigen-binding fragment thereof comprises:
(a) a heavy chain constant region comprising the amino acid sequence set forth in SEQ ID NO: 95, and a light chain constant region comprising the amino acid sequence set forth in SEQ ID NO: 96; or
(b) a heavy chain constant region comprising the amino acid sequence set forth in SEQ ID NO: 95, and a light chain constant region comprising the amino acid sequence set forth in SEQ ID NO: 99.

In certain embodiments, the antibody or antigen-binding fragment thereof comprises a heavy chain variable region that comprises CDR1, CDR2, and CDR3 domains; and a light chain variable region that comprises CDR1, CDR2, and CDR3 domains, wherein the heavy chain variable region and light chain variable region CDR3 domains are selected from the group consisting of:
(a) a heavy chain variable region CDR3 comprising the amino acid sequence set forth in SEQ ID NO: 4 or a conservative modification thereof; and a light chain variable region CDR3 comprising the amino acid sequence set forth in SEQ ID NO: 7 or a conservative modification thereof;
(b) a heavy chain variable region CDR3 comprising the amino acid sequence set forth in SEQ ID NO: 14 or a conservative modification thereof; and a light chain variable region CDR3 comprising the amino acid sequence set forth in SEQ ID NO: 17 or a conservative modification thereof;
(c) a heavy chain variable region CDR3 comprising the amino acid sequence set forth in SEQ ID NO: 24 or a conservative modification thereof; and a light chain variable region CDR3 comprising the amino acid sequence set forth in SEQ ID NO: 27 or a conservative modification thereof;
(d) a heavy chain variable region CDR3 comprising the amino acid sequence set forth in SEQ ID NO: 34 or a conservative modification thereof; and a light chain variable region CDR3 comprising the amino acid sequence set forth in SEQ ID NO: 27 or a conservative modification thereof;
(e) a heavy chain variable region CDR3 comprising the amino acid sequence set forth in SEQ ID NO: 39 or a conservative modification thereof; and a light chain variable region CDR3 comprising the amino acid sequence set forth in SEQ ID NO: 42 or a conservative modification thereof;
(f) a heavy chain variable region CDR3 comprising the amino acid sequence set forth in SEQ ID NO: 48 or a conservative modification thereof; and a light chain variable region CDR3 comprising the amino acid sequence set forth in SEQ ID NO: 51 or a conservative modification thereof;
(g) a heavy chain variable region CDR3 comprising the amino acid sequence set forth in SEQ ID NO: 58 or a conservative modification thereof; and a light chain variable region CDR3 comprising the amino acid sequence set forth in SEQ ID NO: 61 or a conservative modification thereof;
(h) a heavy chain variable region CDR3 comprising the amino acid sequence set forth in SEQ ID NO: 68 or a conservative modification thereof; and a light chain variable region CDR3 comprising the amino acid sequence set forth in SEQ ID NO: 71 or a conservative modification thereof;
(i) a heavy chain variable region CDR3 comprising the amino acid sequence set forth in SEQ ID NO: 77 or a conservative modification thereof; and a light chain variable region CDR3 comprising the amino acid sequence set forth in SEQ ID NO: 80 or a conservative modification thereof; and
(j) a heavy chain variable region CDR3 comprising the amino acid sequence set forth in SEQ ID NO: 87 or a conservative modification thereof; and a light chain variable region CDR3 comprising the amino acid sequence set forth in SEQ ID NO: 42 or a conservative modification thereof.

In certain embodiments, the heavy chain variable region and light chain variable region CDR2 domains of the antibody or antigen-binding fragment thereof are selected from the group consisting of:
(a) a heavy chain variable region CDR2 comprising the amino acid sequence set forth in SEQ ID NO: 3 and a conservative modification thereof; and a light chain variable region CDR2 comprising the amino acid sequence set forth in SEQ ID NO: 6 and a conservative modification thereof;
(b) a heavy chain variable region CDR2 comprising the amino acid sequence set forth in SEQ ID NO: 13 and a conservative modification thereof; and a light chain variable region CDR2 comprising the amino acid sequence set forth in SEQ ID NO: 16 and a conservative modification thereof;
(c) a heavy chain variable region CDR2 comprising the amino acid sequence set forth in SEQ ID NO: 23 and a conservative modification thereof; and a light chain variable region CDR2 comprising the amino acid sequence set forth in SEQ ID NO: 26 and a conservative modification thereof;
(d) a heavy chain variable region CDR2 comprising the amino acid sequence set forth in SEQ ID NO: 33 and a conservative modification thereof; and a light chain variable region CDR2 comprising the amino acid sequence set forth in SEQ ID NO: 26 and a conservative modification thereof;
(e) a heavy chain variable region CDR2 comprising the amino acid sequence set forth in SEQ ID NO: 38 and a conservative modification thereof; and a light chain variable region CDR2 comprising the amino acid sequence set forth in SEQ ID NO: 41 and a conservative modification thereof;
(f) a heavy chain variable region CDR2 comprising the amino acid sequence set forth in SEQ ID NO: 38 and a conservative modification thereof; and a light chain variable region CDR2 comprising the amino acid sequence set forth in SEQ ID NO: 50 and a conservative modification thereof;
(g) a heavy chain variable region CDR2 comprising the amino acid sequence set forth in SEQ ID NO: 57 and a conservative modification thereof; and a light chain variable region CDR2 comprising the amino acid sequence set forth in SEQ ID NO: 60 and a conservative modification thereof;
(h) a heavy chain variable region CDR2 comprising the amino acid sequence set forth in SEQ ID NO: 67 and a conservative modification thereof; and a light chain variable region CDR2 comprising the amino acid sequence set forth in SEQ ID NO: 70 and a conservative modification thereof;
(i) a heavy chain variable region CDR2 comprising the amino acid sequence set forth in SEQ ID NO: 57 and a conservative modification thereof; and a light chain variable region CDR2 comprising the amino acid sequence set forth in SEQ ID NO: 79 and a conservative modification thereof; and
(j) a heavy chain variable region CDR2 comprising the amino acid sequence set forth in SEQ ID NO: 86 and a conservative modification thereof; and a light chain variable region CDR2 comprising the amino acid sequence set forth in SEQ ID NO: 41 and a conservative modification thereof.

In certain embodiments, the heavy chain variable region and light chain variable region CDR1 domains of the antibody or antigen-binding fragment thereof are selected from the group consisting of:
(a) a heavy chain variable region CDR1 comprising the amino acid sequence set forth in SEQ ID NO: 2 and a conservative modification thereof; and a light chain variable region CDR1 comprising the amino acid sequence set forth in SEQ ID NO: 5 and a conservative modification thereof;
(b) a heavy chain variable region CDR1 comprising the amino acid sequence set forth in SEQ ID NO: 12 and a conservative modification thereof; and a light chain variable region CDR1 comprising the amino acid sequence set forth in SEQ ID NO: 15 and a conservative modification thereof;
(c) a heavy chain variable region CDR1 comprising the amino acid sequence set forth in SEQ ID NO: 22 and a conservative modification thereof; and a light chain variable region CDR1 comprising the amino acid sequence set forth in SEQ ID NO: 25 and a conservative modification thereof;
(d) a heavy chain variable region CDR1 comprising the amino acid sequence set forth in SEQ ID NO: 32 and a conservative modification thereof; and a light chain variable region CDR1 comprising the amino acid sequence set forth in SEQ ID NO: 25 and a conservative modification thereof;
(e) a heavy chain variable region CDR1 comprising the amino acid sequence set forth in SEQ ID NO: 37 and a conservative modification thereof; and a light chain variable region CDR1 comprising the amino acid sequence set forth in SEQ ID NO: 40 and a conservative modification thereof;
(f) a heavy chain variable region CDR1 comprising the amino acid sequence set forth in SEQ ID NO: 47 and a conservative modification thereof; and a light chain variable region CDR1 comprising the amino acid sequence set forth in SEQ ID NO: 49 and a conservative modification thereof;
(g) a heavy chain variable region CDR1 comprising the amino acid sequence set forth in SEQ ID NO: 56 and a conservative modification thereof; and a light chain variable region CDR1 comprising the amino acid sequence set forth in SEQ ID NO: 59 and a conservative modification thereof;
(h) a heavy chain variable region CDR1 comprising the amino acid sequence set forth in SEQ ID NO: 66 and a conservative modification thereof; and a light chain variable region CDR1 comprising the amino acid sequence set forth in SEQ ID NO: 69 and a conservative modification thereof;
(i) a heavy chain variable region CDR1 comprising the amino acid sequence set forth in SEQ ID NO: 76 and a conservative modification thereof; and a light chain variable region CDR1 comprising the amino acid sequence set forth in SEQ ID NO: 78 and a conservative modification thereof; and
(j) a heavy chain variable region CDR1 comprising the amino acid sequence set forth in SEQ ID NO: 86 and a conservative modification thereof; and a light chain variable region CDR1 comprising the amino acid sequence set forth in SEQ ID NO: 5 and a conservative modification thereof.

In certain embodiments, one or more of the CDR sequences have up to about 5 amino acid substitutions. In certain embodiments, one or more of the CDR sequences have up to about 3 amino acid substitutions.

In certain embodiments, the anti-CD33 antibody or antigen-binding fragment thereof comprises:
(a) a heavy chain variable region comprising a CDR1 comprising the amino acid sequence set forth in SEQ ID NO: 2, a CDR2 comprising the amino acid sequence set forth in SEQ ID NO: 3, and a CDR3 comprising the amino acid sequence set forth in SEQ ID NO: 4;
(b) a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14;
(c) a heavy chain variable region comprising a CDR1 comprising the amino acid sequence set forth in SEQ ID NO: 22, a CDR2 comprising the amino acid sequence set forth in SEQ ID NO: 23, and a CDR3 comprising the amino acid sequence set forth in SEQ ID NO: 24;
(d) a heavy chain variable region comprising a CDR1 comprising the amino acid sequence set forth in SEQ ID NO: 32, a CDR2 comprising the amino acid sequence set forth in SEQ ID NO: 33, and a CDR3 comprising the amino acid sequence set forth in SEQ ID NO: 34;
(e) a heavy chain variable region comprising a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39;
(f) a heavy chain variable region comprising a CDR1 comprising the amino acid sequence set forth in SEQ ID NO: 47, a CDR2 comprising the amino acid sequence set forth in SEQ ID NO: 38, and a CDR3 comprising the amino acid sequence set forth in SEQ ID NO: 48;
(g) a heavy chain variable region comprising a CDR1 comprising the amino acid sequence set forth in SEQ ID NO: 56, a CDR2 comprising the amino acid sequence set forth in SEQ ID NO:57, and a CDR3 comprising the amino acid sequence set forth in SEQ ID NO: 58;
(h) a heavy chain variable region comprising a CDR1 comprising the amino acid sequence set forth in SEQ ID NO: 66, a CDR2 comprising the amino acid sequence set forth in SEQ ID NO: 67, and a CDR3 comprising the amino acid sequence set forth in SEQ ID NO: 68;
(i) a heavy chain variable region comprising a CDR1 comprising the amino acid sequence set forth in SEQ ID NO: 76, a CDR2 comprising the amino acid sequence set forth in SEQ ID NO: 57, and a CDR3 comprising the amino acid sequence set forth in SEQ ID NO: 77; or
(j) a heavy chain variable region comprising a CDR1 comprising the amino acid sequence set forth in SEQ ID NO: 85, a CDR2 comprising the amino acid sequence set forth in SEQ ID NO: 86, and a CDR3 comprising the amino acid sequence set forth in SEQ ID NO: 87.

In certain embodiments, the anti-CD33 antibody or antigen-binding fragment thereof comprises:
(a) a light chain variable region comprising a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7;
(b) a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17;
(c) a light chain variable region comprising a CDR1 comprising the amino acid sequence set forth in SEQ ID NO: 25, a CDR2 comprising the amino acid sequence set forth in SEQ ID NO: 26, and a CDR3 comprising the amino acid sequence set forth in SEQ ID NO: 27;
(d) a light chain variable region comprising a CDR1 comprising the amino acid sequence set forth in SEQ ID NO: 40, a CDR2 comprising the amino acid sequence set forth in SEQ ID NO: 41, and a CDR3 comprising the amino acid sequence set forth in SEQ ID NO: 42;
(e) a light chain variable region comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51;
(f) a light chain variable region comprising a CDR1 comprising the amino acid sequence set forth in SEQ ID NO: 59, a CDR2 comprising the amino acid sequence set forth in SEQ ID NO: 60, and a CDR3 comprising the amino acid sequence set forth in SEQ ID NO: 61;
(g) a light chain variable region comprising a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71;
(h) a light chain variable region comprising a CDR1 comprising the amino acid sequence set forth in SEQ ID NO: 78, a CDR2 comprising the amino acid sequence set forth in SEQ ID NO: 79, and a CDR3 comprising the amino acid sequence set forth in SEQ ID NO: 80; or
(i) a light chain variable region comprising a CDR1 comprising the amino acid sequence set forth in SEQ ID NO: 5, a CDR2 comprising the amino acid sequence set forth in SEQ ID NO: 41, and a CDR3 comprising the amino acid sequence set forth in SEQ ID NO: 42.

In certain embodiments, the anti-CD33 antibody or antigen-binding fragment thereof comprises:
(a) a heavy chain variable region comprising a CDR1 comprising the amino acid sequence set forth in SEQ ID NO: 2, a CDR2 comprising the amino acid sequence set forth in SEQ ID NO: 3, and a CDR3 comprising the amino acid sequence set forth in SEQ ID NO: 4; and a light chain variable region comprising a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7;
(b) a heavy chain variable region comprising a CDR1 comprising the amino acid sequence set forth in SEQ ID NO: 12, a CDR2 comprising an amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17;
(c) a heavy chain variable region comprising a CDR1 comprising the amino acid sequence set forth in SEQ ID NO: 22, a CDR2 comprising the amino acid sequence set forth in SEQ ID NO: 23, and a CDR3 comprising the amino acid sequence set forth in SEQ ID NO: 24; and a light chain variable region comprising a CDR1 comprising the amino acid sequence set forth in SEQ ID NO: 25, a CDR2 comprising the amino acid sequence set forth in SEQ ID NO: 26, and a CDR3 comprising the amino acid sequence set forth in SEQ ID NO: 27;
(d) a heavy chain variable region comprising a CDR1 comprising the amino acid sequence set forth in SEQ ID NO: 32, a CDR2 comprising the amino acid sequence set forth in SEQ ID NO: 33, and a CDR3 comprising the amino acid sequence set forth in SEQ ID NO: 34; and a light chain variable region comprising a CDR1 comprising the amino acid sequence set forth in SEQ ID NO: 25, a CDR2 comprising the amino acid sequence set forth in SEQ ID NO: 26, and a CDR3 comprising the amino acid sequence set forth in SEQ ID NO: 27;
(e) a heavy chain variable region comprising a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39; and a light chain variable region comprising a CDR1 comprising the amino acid sequence set forth in SEQ ID NO: 40, a CDR2 comprising the amino acid sequence set forth in SEQ ID NO: 41, and a CDR3 comprising the amino acid sequence set forth in SEQ ID NO: 42;
(f) a heavy chain variable region comprising a CDR1 comprising the amino acid sequence set forth in SEQ ID NO: 47, a CDR2 comprising the amino acid sequence set forth in SEQ ID NO: 38, and a CDR3 comprising the amino acid sequence set forth in SEQ ID NO: 48; and a light chain variable region comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51;
(g) a heavy chain variable region comprising a CDR1 comprising the amino acid sequence set forth in SEQ ID NO: 56, a CDR2 comprising the amino acid sequence set forth in SEQ ID NO: 57, and a CDR3 comprising the amino acid sequence set forth in SEQ ID NO: 58; and a light chain variable region comprising a CDR1 comprising the amino acid sequence set forth in SEQ ID NO: 59, a CDR2 comprising the amino acid sequence set forth in SEQ ID NO: 60, and a CDR3 comprising the amino acid sequence set forth in SEQ ID NO: 61;
(h) a heavy chain variable region comprising a CDR1 comprising the amino acid sequence set forth in SEQ ID NO: 66, a CDR2 comprising the amino acid sequence set forth in SEQ ID NO: 67, and a CDR3 comprising the amino acid sequence set forth in SEQ ID NO: 684; and a light chain variable region comprising a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71;
(i) a heavy chain variable region comprising a CDR1 comprising the amino acid sequence set forth in SEQ ID NO: 76, a CDR2 comprising the amino acid sequence set forth in SEQ ID NO: 57, and a CDR3 comprising the amino acid sequence set forth in SEQ ID NO: 77; and a light chain variable region comprising a CDR1 comprising the amino acid sequence set forth in SEQ ID NO: 78, a CDR2 comprising the amino acid sequence set forth in SEQ ID NO: 79, and a CDR3 comprising the amino acid sequence set forth in SEQ ID NO: 80; or
(j) a heavy chain variable region comprising a CDR1 comprising the amino acid sequence set forth in SEQ ID NO: 85, a CDR2 comprising the amino acid sequence set forth in SEQ ID NO: 86, and a CDR3 comprising the amino acid sequence set forth in SEQ ID NO: 87; and a light chain variable region comprising a CDR1 comprising the amino acid sequence set forth in SEQ ID NO: 5, a CDR2 comprising the amino acid sequence set forth in SEQ ID NO: 41, and a CDR3 comprising the amino acid sequence set forth in SEQ ID NO: 42.

In certain embodiments, the sequence of the antibody is in a heavy-light variable chain orientation (V_{H}-V_{L}). In certain embodiments, the antibody or antigen-binding fragment thereof comprises a human variable region framework region.

In certain embodiments, the antibody or antigen-binding fragment thereof is a fully human or an antigen-binding fragment thereof. In certain embodiments, the antibody or antigen-binding fragment thereof is a chimeric antibody or an antigen-binding fragment thereof. In certain embodiments, the antibody or antigen-binding fragment thereof is a humanized antibody or an antigen-binding fragment thereof. In certain embodiments, the antigen-binding fragment of the antibody is a Fab, Fab′, F(ab′)₂, variable fragment (Fv) or a single chain variable fragment (scFv).

In certain embodiments, the antigen-binding fragment of the antibody or antigen-binding fragment thereof is an scFv.

In addition, the presently disclosed subject matter provides antibodies or antigen-binding fragments thereof, which cross-compete for binding to CD33 with any of the above-described antibody or antigen-binding fragment thereof.

The presently disclosed subject matter further provides antibodies or antigen-binding fragments thereof, which binds to the same epitope region on CD33 with any of the above-described antibody or antigen-binding fragment thereof.

The presently disclosed subject matter also provides immunoconjugates comprising the antibody or antigen-binding fragment thereof disclosed herein, linked to a therapeutic agent. In certain embodiments, the therapeutic agent is a drug, a cytotoxin, or a radioactive isotope.

Furthermore, the presently disclosed subject matter provides multi-specific molecules comprising the antibody or antigen-binding fragment thereof disclosed herein, linked to one or more functional moieties. In certain embodiments, the one or more functional moieties have a different binding specificity than the antibody or antigen binding fragment thereof.

Additionally, the presently disclosed subject matter provides compositions comprising the antibody or antigen-binding fragment thereof disclosed herein, the immunoconjugate disclosed herein, or the multi-specific molecule disclosed herein. In certain embodiments, the composition is a pharmaceutical composition that further comprises a pharmaceutically acceptable carrier.

The presently disclosed subject matter provides nucleic acids encoding the antibody or antigen-binding fragment thereof disclosed herein. In addition, the presently disclosed subject matter provides nucleic acids that encodes the heavy chain variable regions of the antibody or antigen-binding fragment thereof disclosed herein. In certain embodiments, the nucleic acid comprises a nucleotide sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the nucleotide sequence set forth in SEQ ID NO: 10, SEQ ID NO: 20, SEQ ID NO: 30, SEQ ID NO: 36, SEQ ID NO: 45, SEQ ID NO: 54, SEQ ID NO: 64, SEQ ID NO: 74, SEQ ID NO: 83, SEQ ID NO: 90, or SEQ ID NO: 93. In certain embodiments, the nucleic acid comprises the nucleotide sequence set forth in SEQ ID NO: 10, SEQ ID NO: 20, SEQ ID NO: 30, SEQ ID NO: 36, SEQ ID NO: 45, SEQ ID NO: 54, SEQ ID NO: 64, SEQ ID NO: 74, SEQ ID NO: 83, SEQ ID NO: 90, or SEQ ID NO: 93.

Furthermore, the presently disclosed subject matter provides nucleic acids that encodes the light chain variable regions of the antibody or antigen-binding fragment thereof disclosed herein. In certain embodiments, the nucleic acid comprises a nucleotide sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the nucleotide sequence set forth in SEQ ID NO: 11, SEQ ID NO: 21, SEQ ID NO: 31, SEQ ID NO: 46, SEQ ID NO: 55, SEQ ID NO: 65, SEQ ID NO: 75, SEQ ID NO: 84, SEQ ID NO: 91, or SEQ ID NO: 94. In certain embodiments, the nucleic acid comprises the nucleotide sequence set forth in SEQ ID NO: 11, SEQ ID NO: 21, SEQ ID NO: 31, SEQ ID NO: 46, SEQ ID NO: 55, SEQ ID NO: 65, SEQ ID NO: 75, SEQ ID NO: 84, SEQ ID NO: 91, or SEQ ID NO: 94.

Furthermore, the presently disclosed subject matter provides vectors comprising such nucleic acid molecules, and host cells comprising such vectors.

The presently disclosed subject matter provides methods for detecting CD33 in a cell, a tissue, or a blood sample. In certain embodiments, the method comprises: contacting a cell, a tissue, or a blood sample with the antibody or antigen-binding fragment thereof disclosed herein, wherein the antibody or antigen-binding fragment thereof comprises a detectable label; and determining the amount of the labeled antibody or antigen-binding fragment thereof bound to the cell, tissue, blood sample by measuring the amount of detectable label associated with the cell, tissue, or blood sample, wherein the amount of bound antibody or antigen-binding fragment thereof indicates the amount of CD33 in the cell, tissue, or blood sample.

Furthermore, the presently disclosed subject matter provides various methods of using the antibodies and antigen-binding fragments thereof, immunoconjugates, multi-specific molecules, or compositions disclosed herein. The presently disclosed subject matter provides methods of treating or ameliorating a disease or disorder associated with CD33 in a subject. In certain embodiments, the method comprises administering to the subject the antibody or antigen-binding fragment thereof, the immunoconjugate, multi-specific molecule, or composition disclosed herein. In certain embodiments, the disease or disorder expresses CD33. In certain embodiments, the disease or disorder is associated with overexpression of CD33. In certain embodiments, the disease or disorder is tumor.

The presently disclosed subject matter also provides methods of reducing tumor burden in a subject. In certain embodiments, the method comprises administering to the subject the antibody or antigen-binding fragment thereof, the immunoconjugate, multi-specific molecule, or composition disclosed herein. In certain embodiments, the method reduces the number of the tumor cells, reduces the tumor size, and/or eradicates the tumor in the subject.

In addition, the presently disclosed subject matter also provides methods of treating and/or preventing a tumor in a subject. In certain embodiments, the method comprises administering to the subject the antibody or antigen-binding fragment thereof, the immunoconjugate, multi-specific molecule, or composition disclosed herein.

In addition, the presently disclosed subject matter also provides methods of increasing or lengthening survival of a subject having a tumor. In certain embodiments, the method comprises administering to the subject the antibody or antigen-binding fragment thereof, the immunoconjugate, multi-specific molecule, or composition disclosed herein. In certain embodiments, the method reduces or eradicates tumor burden in the subject.

In certain embodiments, the tumor is cancer. In certain embodiments, the tumor is hematological cancer, or solid tissue cancer. In certain embodiments, the hematological cancer is selected from the group consisting of acute myeloid leukemia (AML), myelodysplastic syndromes (MDS), acute lymphoblastic leukemia (ALL), chronic lymphocytic leukemia (CLL), myeloproliferative neoplasms (MPNs), and chronic myeloid neoplasms. In certain embodiments, the hematological cancer is acute myeloid leukemia (AML). In certain embodiments, the subject is a human.

Furthermore, the presently disclosed subject matter provides kits for treating or ameliorating a disease or disorder in a subject, treating or ameliorating a disease or disorder in a subject, reducing tumor burden in a subject, treating and/or preventing a tumor in a subject, and/or increasing or lengthening survival of a subject having a tumor, comprising the antibody or antigen-binding fragment thereof, the immunoconjugate thereof, the multi-specific molecule thereof, or the composition disclosed herein. In certain embodiments, the kit further comprises written instructions for using the antibody or antigen-binding fragment thereof, the immunoconjugate thereof, the multi-specific molecule thereof, or the composition thereof disclosed herein for treating or ameliorating a disease or disorder in a subject, treating or ameliorating a disease or disorder in a subject, reducing tumor burden in a subject, treating and/or preventing a tumor in a subject, and/or increasing or lengthening survival of a subject having a tumor.

### 4. BRIEF DESCRIPTION OF THE FIGURES

The following Detailed Description, given by way of example, but not intended to limit the invention to specific embodiments described, may be understood in conjunction with the accompanying drawings.

FIGS. 1A and 1B depict CD33 domain architecture. FIG. 1A depicts a schematic of full-length CD33 (CD33M) consisting of two Ig-like domains (IgV and IgC2) in the extracellular region, a single transmembrane segment, an immunoreceptor tyrosine-based inhibitory motif (ITIM) and an ITIM-like motif in the cytosolic region. In a common CD33 variant (CD33m), the IgV domain is missing due to alternate splicing. FIG. 1B depicts the CD33 gene structure with and without alternate splicing. Removal of exon 2 results in the loss of the IgV domain. Several antibodies that have been advanced to the clinic with various formats (CAR, BITE, radiotherapy) target the IgV domain. GO, gemtuzumab ozogamicin.

FIG. 2 depicts candidate antibodies binding to CD33 protein. Binding of 10 selected clones tested by ELISA on mouse (moCD33) and human (huCD33) proteins. All 10 clonal mAbs showed binding to human CD33 but not mouse CD33. Anti-His, anti-CD33 and irrelevant protein with His were used as controls.

FIG. 3 depicts the binding of candidate antibodies to recombinant CD33 protein. Recombinant human, cynomolgus and mouse CD33-ECD-His were captured by Ni-plates and antibodies added at 10 μg/mL concentration in triplicates. HRP conjugated anti-human Fc secondary antibody was used for detection.

FIGS. 4A and 4B depict the binding of exemplified monoclonal anti-CD33 antibodies disclosed herein to CD33 expressed on cell surface. FIG. 4A depicts the binding of monoclonal antibodies TDI-Y-006, TDI-Y-007 and 1J19 on CD33-overexpressing 3T3 cells. FIG. 4B depicts the binding of monoclonal antibodies TDI-Y-006, TDI-Y-007 and 1J19 to U937, an AML line with endogenous CD33 expression. Binding was tested at increasing concentration by flow cytometry. Human IgG1 (HuIgG1) isotype was included as a control.

### 5. DETAILED DESCRIPTION OF EMBODIMENTS OF THE INVENTION

The presently disclosed subject matter provides anti-CD33 antibodies. Non-limiting embodiments of the present disclosure are described by the present specification and Examples.

For purposes of clarity of disclosure and not by way of limitation, the detailed description is divided into the following subsections:
5.1. Definitions;
5.2. CD33;
5.3. Anti-CD33 Antibodies;
5.4. Nucleic Acids encoding the Antibodies or Antigen-binding Fragments;
5.5. Pharmaceutical Compositions and Methods of Treatment;
5.6. Diagnostic and Prognostic Methods;
5.7. Kits; and
5.8. Exemplary Embodiments

### 5.1. Definitions

In the description that follows, certain conventions will be followed as regards the usage of terminology. Generally, terms used herein are intended to be interpreted consistently with the meaning of those terms as they are known to those of skill in the art.

“Antibody” and “antibodies” as those terms are known in the art refer to antigen binding proteins of the immune system. The term “antibody” as referred to herein includes whole, full length antibodies having an antigen-binding region, and any fragment thereof in which the “antigen-binding fragment” or “antigen-binding region” is retained, or single chains, for example, single chain variable fragment (scFv), thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (CH) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the presently disclosed subject matter may include amino acid residues not encoded by human germline immunoglobulin sequences (e.g., mutations introduced by random or site-specific mutagenesis in vitro or by somatic mutation in vivo).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed subject matter may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to CD33” is intended to refer to an antibody that binds to CD33 (e.g., human CD33, e.g., soluble human CD33) with a dissociation constant (K_{D}) of about 1×10⁻⁸ M or less, about 5×10⁻⁹ M or less, about 1×10⁻⁹ M or less, about 5×10⁻¹⁰ M or less, about 1×10⁻¹⁰ M or less, about 5×10⁻¹¹ M or less, or about 1×10⁻¹¹ M or less.

An “antibody that competes for binding” or “antibody that cross-competes for binding” with a reference antibody for binding to an antigen, e.g., CD33, refers to an antibody that blocks binding of the reference antibody to the antigen (e.g., CD33) in a competition assay by 50% or more, and conversely, the reference antibody blocks binding of the antibody to the antigen (e.g., CD33) in a competition assay by 50% or more. An exemplary competition assay is described in “Antibodies”, Harlow and Lane (Cold Spring Harbor Press, Cold Spring Harbor, NY).

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term” an antibody which binds specifically to an antigen (e.g., a CD33 polypeptide).”

The term “antigen-binding fragment” or “antigen-binding region” of an antibody, as used herein, refers to that region or fragment of the antibody that binds to the antigen and which confers antigen specificity to the antibody; fragments of antigen-binding proteins, for example, antibodies includes one or more fragments of an antibody that retain the ability to specifically bind to an antigen (e.g., a CD33 polypeptide). It has been shown that the antigen-binding function of an antibody can be performed by fragments of a full-length antibody. Examples of antigen-binding fragments encompassed within the term “antibody fragments” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., Nature 1989; 341:544-546), which consists of a V_{H} domain; and an isolated complementarity determining region (CDR).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules. These are known as single chain Fv (scFv); see e.g., Bird et al., Science (1988); 242:423-426; and Huston et al., Proc Natl Acad Sci (1998); 85:5879-5883. These antibody fragments are obtained using conventional techniques known to those of skill in the art, and the fragments are screened for utility in the same manner as are intact antibodies.

An “antibody” or “antigen-binding protein” is one which has been identified and separated and/or recovered from a component of its natural environment. “Synthetic antibodies” or “recombinant antibodies” are generally generated using recombinant technology or using peptide synthetic techniques known to those of skill in the art.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::V_{L} heterodimer. The heavy (V_{H}) and light chains (V_{L}) are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain.

Non-limiting examples of linkers are disclosed in Shen et al., Anal Chem (2008); 80(6):1910-1917 and WO 2014/087010, the contents of which are hereby incorporated by reference in their entireties. In certain embodiments, the linker is a G4S linker. In certain embodiments, the linker comprises or consists of the amino acid sequence set forth in SEQ ID NO: 104, which is provided below:

[SEQ ID NO: 104]
GGGGSGGGGSGGGSGGGGS

In certain embodiments, the linker comprises or consists of the amino acid sequence set forth in SEQ ID NO: 105, which is provided below:

[SEQ ID NO: 105]
GGGGSGGGGSGGGGS

In certain embodiments, the linker comprises or consists of the amino acid sequence set forth in SEQ ID NO: 106, which is provided below:

[SEQ ID NO: 106]
GGGGSGGGGSGGGGSGGGSGGGGS

In certain embodiments, the linker comprises or consists of the amino acid sequence set forth in SEQ ID NO: 107, which is provided below:

[SEQ ID NO: 107]
GGGGSGGGGSGGGGSGGGGSGGGSGGGGS

In certain embodiments, the linker comprises or consists of the amino acid sequence set forth in SEQ ID NO: 108, which is provided below:

[SEQ ID NO: 108]
GGGGS

In certain embodiments, the linker comprises or consists of the amino acid sequence set forth in SEQ ID NO: 109, which is provided below:

[SEQ ID NO: 109]
GGGGSGGGGS

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. (Proc. Nat. Acad. Sci. USA, 1988; 85:5879-5883). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008; 27(6):455-51; Peter et al., J Cachexia Sarcopenia Muscle 2012 Aug. 12; Shieh et al., J Imunol 2009; 183(4):2277-85; Giomarelli et al., Thromb Hacmost 2007; 97(6):955-63; Fife eta., J Clin Invst 2006; 116(8):2252-61; Brocks et al., Immunotechnology 1997; 3(3): 173-84; Moosmayer et al., Ther Immunol 1995; 2 (10:31-40). Agonistic scFvs having stimulatory activity have been described (see, e.g., Peter et al., J Biol Chem 2003; 25278(38):36740-7; Xie et al., Nat Biotech 1997; 15(8): 768-71; Ledbetter et al., Crit Rev Immunol 1997; 17 (5-6):427-55; Ho et al., BioChim Biophys Acta 2003; 1638(3):257-66).

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)₂” fragment can be split into two individual Fab′ fragments.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

“CDRs” are defined as the complementarity determining region amino acid sequences of an antibody which are the hypervariable regions of immunoglobulin heavy and light chains. Sec, e.g., Kabat et al., Sequences of Proteins of Immunological Interest, 4th U. S. Department of Health and Human Services, National Institutes of Health (1987), or IMGT numbering system (Lefranc, The Immunologist (1999); 7:132-136; Lefranc et al., Dev. Comp. Immunol. (2003); 27:55-77). The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise three heavy chain and three light chain CDRs or CDR regions in the variable region. CDRs provide the majority of contact residues for the binding of the antibody to the antigen or epitope region. In certain embodiments, the CDRs are identified according to the IMGT system. In certain embodiments, the CDRs are identified using the IMGT numbering system accessible at http://www.imgt.org/IMGT_vquest/input.

The terms “isolated” denotes a degree of separation from original source or surroundings. An “isolated antibody” is one which has been separated from a component of its natural environment. In certain embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr (2007); B 848:79-87.

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

An “isolated nucleic acid encoding an antibody” (including references to a specific antibody, e.g. an anti-KLB antibody) refers to one or more nucleic acid molecules encoding antibody heavy and light chains (or fragments thereof), including such nucleic acid molecule(s) in a single vector separate vectors, and such nucleic acid molecule(s) present at one or more locations in a host cell.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

An “immunoconjugate” is an antibody conjugated to one or more heterologous molecule(s), including, but not limited to, a cytotoxic agent.

As used herein, the term “derivative” refers to a compound that is derived from some other compound and maintains its general structure. For example, but without any limitation, trichloromethane (chloroform) is a derivative of methane.

An “effective amount” (or, “therapeutically effective amount”) is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition, and the form and effective concentration of the cells administered.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters; guinea pigs; rabbits; dogs; cats; sheep; pigs; goats; cattle; horses; and non-human primates such as apes and monkeys.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In certain embodiments, antibodies of the presently disclosed subject matter are used to delay development of a disease or to slow the progression of a disease, e.g., a tumor, e.g., a tumor associated with CD33.

The terms “comprises”, “comprising”, and are intended to have the broad meaning ascribed to them in U.S. Patent Law and can mean “includes”, “including” and the like.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As described herein, any concentration range, percentage range, ratio range or integer range is to be understood to include the value of any integer within the recited range and, when appropriate, fractions thereof (such as one tenth and one hundredth of an integer), unless otherwise indicated.

Other aspects of the presently disclosed subject matter are described in the following disclosure and are within the ambit of the presently disclosed subject matter.

### 5.2. CD33

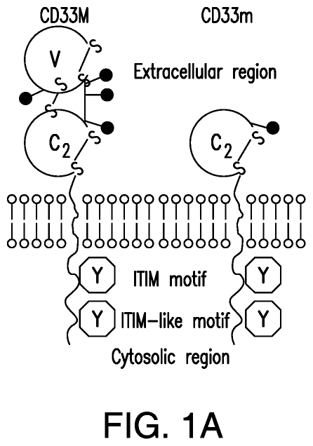

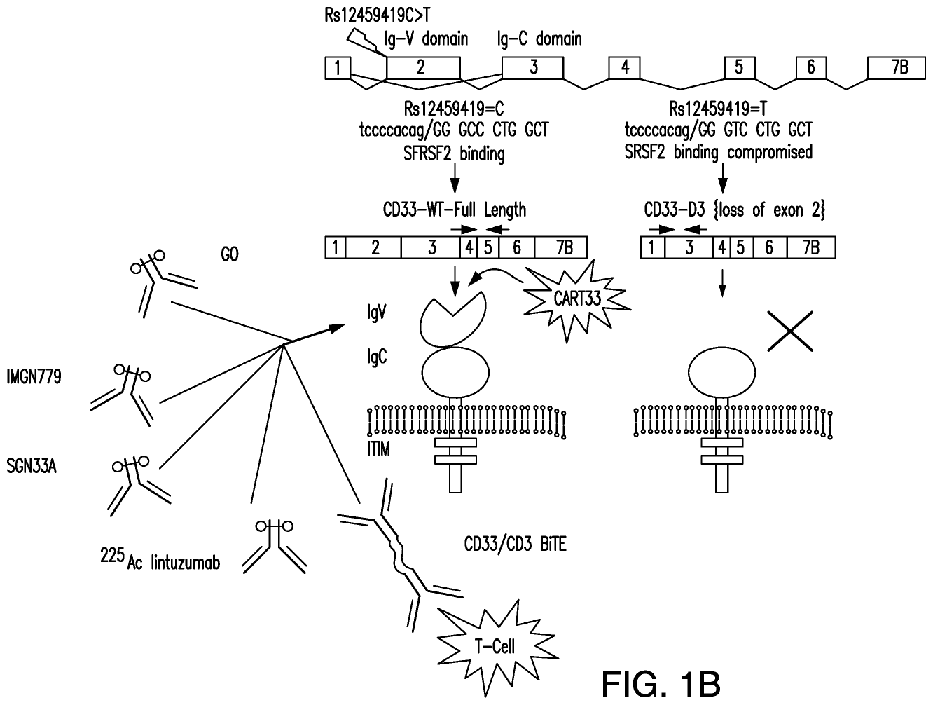

CD33 is a single pass transmembrane molecule and a member of the sialic acid-binding immunoglobulin (Ig)-like lectin (Siglec) family. CD33 consists of two extracellular domains with immunoglobulin-like folds, IgV and IgC2 (see FIG. 1A). CD33 has 3 isoforms produced via alternate splicing, with isoform 3 missing the IgV domain (Ehninger et al., Blood Cancer J 4, e218 (2014); Sanford et al., Leuk Lymphoma 57, 1965-1968 (2016); and Haubner et al., Leukemia 33, 64-74 (2019)) Recent studies showed that about 50% of AML patients have a CD33 single-nucleotide polymorphism (SNP) (rs12459419 C>T) that leads to the expression of an alternatively spliced CD33 isoform lacking exon 2, resulting in the elimination of the IgV domain (Bakker et al., Cancer Res 64, 8443-8450 (2004)). In these patients, gemtuzumab ozogamicin (GO), an antibody-drug conjugate (ADC) targeting CD33, had no impact and increased relapse risk likely due to the inability of this ADC to kill AML cells expressing this IgV-lacking CD33 isoform. This issue is bound to be encountered by all the currently clinically available CD33-targeting products given their epitopes in the IgV domain (see FIG. 1B) (Perna et al., Cancer Cell 32, 506-519 e505 (2017)).

In certain embodiments, the presently disclosed anti-CD33 antibodies or antigen-binding fragments thereof bind to human CD33. In certain embodiments, the human CD33 comprises or consists of the amino acid sequence with a UniProt Reference No: P20138-1 (SEQ ID NO: 1) or a fragment thereof. SEQ ID NO: 1 is provided below. In certain embodiments, the human CD33 comprises an extracellular domain, a transmembrane domain, and a cytoplasmic domain. In certain embodiments, the extracellular domain comprises or consists of amino acids 18 to 259 of SEQ ID NO: 1. In certain embodiments, the transmembrane domain comprises or consists of amino acids 260 to 282 of SEQ ID NO: 1. In certain embodiments, the cytoplasmic domain comprises or consists of amino acids 283 to 364 of SEQ ID NO: 1.

[SEQ ID NO: 1]
MPLLLLLPLL WAGALAMDPN FWLQVQESVT VQEGLCVLVP
CTFFHPIPYY DKNSPVHGYW FREGAIISRD SPVATNKLDQ
EVQEETQGRF RLLGDPSRNN CSLSIVDARR RDNGSYFFRM
ERGSTKYSYK SPQLSVHVTD LTHRPKILIP GTLEPGHSKN
LTCSVSWACE QGTPPIFSWL SAAPTSLGPR TTHSSVLIIT
PRPQDHGTNL TCQVKFAGAG VTTERTIQLN VTYVPQNPTT
GIFPGDGSGK QETRAGVVHG AIGGAGVTAL LALCLCLIFF
IVKTHRRKAA RTAVGRNDTH PTTGSASPKH QKKSKLHGPT
ETSSCSGAAP TVEMDEELHY ASLNFHGMNP SKDTSTEYSE VRTQ

In certain embodiments, the CD33 comprises or consists of an amino acid sequence that is at least about 80%, at least about 85%, at least about 90%, at least about 95%, at least about 96%, at least about 97%, at least about 98%, or at least about 99%, at least about 100% identical to the amino acid sequence set forth in SEQ ID NO: 1 or a fragment thereof.

In certain embodiments, the anti-CD33 antibodies or antigen-binding fragments thereof bind to a portion of human CD33. In certain embodiments, the anti-CD33 antibodies or antigen-binding fragments thereof bind to the extracellular domain of CD33. In certain embodiments, the anti-CD33 antibodies or antigen-binding fragments thereof bind to amino acids 18 to 259 of SEQ ID NO: 1.

### 5.3. Anti-CD33 Antibodies

The antibodies of the presently disclosed subject matter are characterized by particular functional features or properties of the antibodies. For example, the antibodies bind specifically to CD33 (e.g., bind to human CD33).

In certain embodiments, a presently disclosed antibody or antigen-binding fragment binds to CD33 (e.g., human CD33) with a binding affinity, for example with a dissociation constant (K_{D}) of 1×10⁻⁸ M or less, e.g., about 1×10⁻⁸ M or less, about 5×10⁻⁹ M or less, about 1×10⁻⁹ M or less, about 5×10⁻¹⁰ M or less, about 1×10⁻¹⁰ M or less, or about 1×10⁻¹¹ M or less. In certain embodiments, the presently disclosed anti-CD33 antibody or antigen-binding fragment thereof binds to CD33 (e.g., human CD33, e.g. soluble human CD33) with a dissociation constant (K_{D}) of about 5×10⁻⁹ M or less. In certain embodiments, the presently disclosed anti-CD33 antibody or antigen-binding fragment thereof binds to CD33 (e.g., human CD33, e.g. soluble human CD33) with a dissociation constant (K_{D}) of about 5×10⁻⁹ M. In certain embodiments, the presently disclosed anti-CD33 antibody or antigen-binding fragment thereof binds to CD33 (e.g., human CD33, e.g. soluble human CD33) with a dissociation constant (K_{D}) of about 1×10⁻⁹ M. In certain embodiments, the presently disclosed anti-CD33 antibody or antigen-binding fragment thereof binds to CD33 (e.g., human CD33, e.g. soluble human CD33) with a dissociation constant (K_{D}) of between about 1×10⁻⁹ M and about 5×10⁻⁹ M. In certain embodiments, the presently disclosed anti-CD33 antibody or antigen-binding fragment thereof binds to CD33 (e.g., human CD33, e.g. soluble human CD33) with a dissociation constant (K_{D}) of between about 1×10⁻⁹ M and about 2×10⁻⁹ M. In certain embodiments, the presently disclosed anti-CD33 antibody or antigen-binding fragment thereof binds to CD33 (e.g., human CD33, e.g. soluble human CD33) with a dissociation constant (K_{D}) of about 5×10⁻⁹ M. In certain embodiments, the presently disclosed anti-CD33 antibody or antigen-binding fragment thereof binds to CD33 (e.g., human CD33, e.g. soluble human CD33) with a dissociation constant (K_{D}) of about 1×10⁻⁹ M.

In certain embodiments, a presently disclosed antibody or antigen-binding fragment binds to a cell expressing CD33 (e.g., an AML cell expressing CD33) with a Half maximal Effective Concentration (EC₅₀) value of from about 1 nM to about 50 nM, from about 5 nM to about 50 nM, from about 10 nM to about 50 nM, from about 20 nM to about 50 nM, from about 30 nM to about 50 nM, from about 40 nM to about 50 nM, or greater than about 50 nM. In certain embodiments, a presently disclosed antibody or antigen-binding fragment binds to a cell expressing CD33 (e.g., an AML cell expressing CD33) with an EC₅₀ value from about 1 nM to about 5 nM. In certain embodiments, a presently disclosed antibody or antigen-binding fragment binds to a cell expressing CD33 (e.g., an AML cell expressing CD33) with an EC₅₀ value of about 2 nM. In certain embodiments, a presently disclosed antibody or antigen-binding fragment binds to a cell expressing CD33 (e.g., an AML cell expressing CD33) with an EC₅₀ value of about 2.16 nM. In certain embodiments, a presently disclosed antibody or antigen-binding fragment binds to a cell expressing CD33 (e.g., an AML cell expressing CD33) with an EC₅₀ value from about 5 nM to about 10 nM. In certain embodiments, a presently disclosed antibody or antigen-binding fragment binds to a cell expressing CD33 (e.g., an AML cell expressing CD33) with an EC₅₀ value of about 10 nM. In certain embodiments, a presently disclosed antibody or antigen-binding fragment binds to a cell expressing CD33 (e.g., an AML cell expressing CD33) with an EC₅₀ value of about 8.5 nM. In certain embodiments, a presently disclosed antibody or antigen-binding fragment binds to a cell expressing CD33 (e.g., an AML cell expressing CD33) with an EC₅₀ value from about 40 nM to about 50 nM. In certain embodiments, a presently disclosed antibody or antigen-binding fragment binds to a cell expressing CD33 (e.g., an AML cell expressing CD33) with an EC₅₀ value of about 45 nM. In certain embodiments, a presently disclosed antibody or antigen-binding fragment binds to a cell expressing CD33 (e.g., an AML cell expressing CD33) with an EC₅₀ value of about 45 nM.

The heavy and light chains of a presently disclosed antibody or antigen-binding fragment can be full-length (e.g., an antibody can include at least one (e.g., one or two) complete heavy chains, and at least one (e.g., one or two) complete light chains) or can include an antigen-binding fragment (a Fab, F(ab′)2, Fv or a single chain Fv fragment (“scFv”)). In certain embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4. In certain embodiments, the immunoglobulin isotype is IgG1 (e.g., human IgG1). The choice of antibody isotype can depend on the immune effector function that the antibody is designed to elicit. In certain embodiments, the antibody light chain constant region is chosen from, e.g., kappa or lambda, particularly kappa.

In constructing a recombinant immunoglobulin, appropriate amino acid sequences for constant regions of various immunoglobulin isotypes and methods for the production of a wide array of antibodies are known to those of skill in the art.

5.3.1. Single-Chain Variable Fragments (scFvs)

In certain embodiments, the presently disclosed subject matter includes antibodies or antigen-binding fragments thereof that have the scFv sequence fused to one or more constant domains to form an antibody with an Fc region of a human immunoglobulin to yield a bivalent protein, increasing the overall avidity and stability of the antibody. In addition, the Fc portion allows the direct conjugation of other molecules, including but not limited to fluorescent dyes, cytotoxins, radioisotopes etc. to the antibody for example, for use in antigen quantitation studies, to immobilize the antibody for affinity measurements, for targeted delivery of a therapeutic agent, to test for Fc-mediated cytotoxicity using immune effector cells and many other applications.

The results presented here highlight the specificity, sensitivity and utility of the presently disclosed antibodies or antigen-binding fragments in targeting a CD33 polypeptide (e.g., a human CD33 polypeptide).

In certain embodiments, the anti-CD33 scFv is an scFv-Fc fusion protein or a full-length human IgG with V_{H} and V_{L} regions or CDRs selected from Table 1. In certain embodiments, the anti-CD33 scFv comprises a V_{H} comprising the amino acid sequence set forth in SEQ ID NO: 8. An exemplary nucleotide sequence encoding the amino acid sequence of SEQ ID NO: 8 is set forth in SEQ ID NO: 10. In certain embodiments, the anti-CD33 scFv comprises a V_{L} comprising the amino acid sequence set forth in SEQ ID NO: 9. An exemplary nucleotide sequence encoding the amino acid sequence of SEQ ID NO: 9 is set forth in SEQ ID NO: 11. SEQ ID NO: 8-11 are provided in Table 1. In certain embodiments, the scFv is designated as “3-P14”.

In certain embodiments, the anti-CD33 scFv comprises a V_{H} comprising the amino acid sequence set forth in SEQ ID NO: 8 and a V_{L} comprising the amino acid sequence set forth in SEQ ID NO: 9.

In certain embodiments, the anti-CD33 scFv comprises a V_{H} comprising a CDR1 comprising the amino acid sequence set forth in SEQ ID NO: 2 or a conservative modification thereof, a CDR2 comprising the amino acid sequence set forth in SEQ ID NO: 3 or a conservative modification thereof, and a CDR3 comprising the amino acid sequence set forth in SEQ ID NO: 4 or a conservative modification thereof. SEQ ID NOs: 2-4 are provided in Table 1.

In certain embodiments, the anti-CD33 scFv comprises a V_{L} comprising a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 7 or a conservative modification thereof. SEQ ID NOs: 4-6 are provided in Table 1.

In certain embodiments, the anti-CD33 scFv comprises a V_{H} comprising a CDR1 comprising the amino acid sequence set forth in SEQ ID NO: 2 or a conservative modification thereof, a CDR2 comprising the amino acid sequence set forth in SEQ ID NO: 3 or a conservative modification thereof, and a CDR3 comprising the amino acid sequence set forth in SEQ ID NO: 4 or a conservative modification thereof; and a V_{L} comprising a CDR1 comprising the amino acid sequence set forth in SEQ ID NO: 5 or a conservative modification thereof, a CDR2 comprising the amino acid sequence set forth in SEQ ID NO: 6 or a conservative modification, and a CDR3 comprising the amino acid sequence set forth in SEQ ID NO: 7 or a conservative modification thereof.

In certain embodiments, the anti-CD33 scFv comprises a V_{H} comprising a CDR1 comprising the amino acid sequence set forth in SEQ ID NO: 2, a CDR2 comprising the amino acid sequence set forth in SEQ ID NO: 3, and a CDR3 comprising the amino acid sequence set forth in SEQ ID NO: 4; and a V_{L} comprising a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7.

In certain embodiments, the anti-CD33 scFv comprises a V_{H} comprising the amino acid sequence set forth in SEQ ID NO: 8, and a V_{L} comprising the amino acid sequence set forth in SEQ ID NO: 9. In certain embodiments, the V_{H} and V_{L} are linked via a linker. In certain embodiments, the linker comprises the amino acid sequence set forth in SEQ ID NO: 105.

In certain embodiments, the variable regions are linked one after another such that a heavy chain variable region (V_{H}) is position at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 1

| CDRs | 1 | 2 | 3 |
|---|---|---|---|
| V_{H} | GFTFSTYA | ISGRGGST | AGRGDYYYYYGMDV |
| | [SEQ ID NO: 2] | [SEQ ID NO: 3] | [SEQ ID NO: 4] |
| V_{L} | QSLVYSDGNTY | KIS | MQSTQFPHT |
| | [SEQ ID NO: 5] | [SEQ ID NO: 6] | [SEQ ID NO: 7] |

| | |
|---|---|
| Full V_{H} | EVQLLESGGGLVQPGGSLRLSCAASGFTFSTYAMSWVRQAPGKGLEWVS |
| | AISGRGGSTYYTDSVKGRFTISRDNSKNTVSLQMNSLRAEDTAVYYCAG |
| | RGDYYYYYGMDVWGQGTTVTVSA [SEQ ID NO: 8] |
| Full V_{L} | DIVMTQSPLSSPVTLGQPASFSCRSSQSLVYSDGNTYLSWLQQRPGQPP |
| | RLLIYKISNRFSGVPDRFSGSGAGTDFTLKISRVEAEDVGVYYCMQSTQ |
| | FPHTFGQGTKLEIK |
| | [SEQ ID NO: 9] |
| DNA for | GAGGTGCAGCTGTTGGAGTCTGGGGGAGGCTTGGTCCAGCCTGGGGGGT |
| Full V_{H} | CCCTGAGACTCTCCTGTGCAGCCTCTGGATTCACCTTTAGCACCTATGC |
| | CATGAGCTGGGTCCGCCAGGCTCCAGGGAAGGGGCTGGAGTGGGTCTCA |
| | GCTATTAGTGGTCGTGGTGGTAGCACATACTACACAGACTCCGTGAAGG |
| | GCCGGTTCACCATCTCCAGAGACAATTCCAAGAACACGGTGTCTCTGCA |
| | AATGAACAGCCTGAGAGCCGAGGACACGGCCGTATATTACTGTGCGGGC |
| | CGGGGAGATTACTACTACTACTACGGTATGGACGTCTGGGGCCAAGGGA |
| | CCACGGTCACCGTCTCCGCA [SEQ ID NO: 10] |
| DNA for | GATATTGTGATGACCCAGAGTCCACTCTCCTCACCTGTCACCCTTGGAC |
| Full V_{L} | AGCCGGCCTCCTTCTCCTGCAGGTCTAGTCAAAGCCTCGTATACAGTGA |
| | TGGAAACACCTACTTGAGTTGGCTTCAGCAGAGGCCAGGCCAGCCTCCA |
| | AGACTCCTAATTTATAAGATTTCTAACCGGTTCTCTGGGGTCCCAGACA |
| | GATTCAGTGGCAGTGGGGCAGGGACAGATTTCACACTGAAAATCAGCAG |
| | GGTGGAAGCTGAGGATGTCGGGGTTTATTACTGCATGCAATCTACACAA |
| | TTTCCTCACACTTTTGGCCAGGGGACCAAGCTGGAGATCAAA [SEQ |
| | ID NO: 11] |

In certain embodiments, the anti-CD33 scFv is an scFv-Fc fusion protein or a full-length human IgG with V_{H} and V_{L} regions or CDRs selected from Table 2. In certain embodiments, the anti-CD33 scFv comprises a V_{H} comprising the amino acid sequence set forth in SEQ ID NO: 18, as shown in Table 2. An exemplary nucleotide sequence encoding the amino acid sequence of SEQ ID NO: 18 is set forth in SEQ ID NO: 20. In certain embodiments, the anti-CD33 scFv comprises a V_{L} comprising the amino acid sequence set forth in SEQ ID NO: 19. An exemplary nucleotide sequence encoding the amino acid sequence of SEQ ID NO: 19 is set forth in SEQ ID NO: 21. In certain embodiments, the anti-CD33 scFv comprises a V_{H} comprising the amino acid sequence set forth in SEQ ID NO: 18 and a V_{L} comprising the amino acid sequence set forth in SEQ ID NO: 19. SEQ ID NO: 18-21 are provided in Table 2. In certain embodiments, the scFv is designated as “4-B2”.

In certain embodiments, the anti-CD33 scFv comprises a V_{H} comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 2.

In certain embodiments, the anti-CD33 scFv comprises a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 2.

In certain embodiments, the anti-CD33 scFv comprises a V_{H} comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof; and a V_{L} comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification.

In certain embodiments, the anti-CD33 scFv comprises a V_{H} C comprising a DR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the anti-CD33 scFv comprises a V_{H} comprising the amino acid sequence set forth in SEQ ID NO: 18, and a V_{L} comprising the amino acid sequence set forth in SEQ ID NO: 19. In certain embodiments, the V_{H} and VI are linked via a linker. In certain embodiments, the linker comprises the amino acid sequence set forth in SEQ ID NO: 105.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 2

| CDRs | 1 | 2 | 3 |
|---|---|---|---|
| V_{H} | GFIFSSNA | ISGYGGNT | AKWGTYIVGATGDY |
| | [SEQ ID NO: 12] | [SEQ ID NO: 13] | [SEQ ID NO: 14] |
| V_{L} | SNDVGGYNY | EVS | SSYAGSNNWV |
| | [SEQ ID NO: 15] | [SEQ ID NO: 16] | [SEQ ID NO: 17] |

| | |
|---|---|
| Full V_{H} | EVHLLESGGGLVQPGGSLRLSCAASGFIFSSNAMSWVRQAPGKGLEWVS |
| | AISGYGGNTYYADSVKGRFTISRDNSKNTLYLQMNSLRAEDTAVYYCAK |
| | WGTYIVGATGDYWGQGTLVTVSS [SEQ ID NO: 18] |
| Full V_{L} | QSALTQPPSASGSPGQSVTISCTGTSNDVGGYNYVSWYQQHPGKAPKLL |
| | IYEVSKRPSGVPDRFSGSQSGNTASLTVSGLQAEDEADYYCSSYAGSNN |
| | WVFGGGTKLTVL |
| | [SEQ ID NO: 19] |
| DNA for | GAGGTGCACCTGTTGGAGTCTGGGGGAGGCTTGGTACAGCCTGGGGGGT |
| Full V_{H} | CCCTGAGACTCTCCTGTGCAGCCTCTGGATTCATCTTTAGCAGCAATGC |
| | CATGAGCTGGGTCCGCCAGGCTCCAGGGAAGGGACTGGAGTGGGTCTCA |
| | GCTATTAGTGGTTATGGTGGTAACACATACTACGCAGACTCCGTGAAGG |
| | GCCGGTTCACCATCTCCAGAGACAATTCCAAGAACACGCTATATCTGCA |
| | AATGAACAGCCTGAGAGCCGAGGACACGGCCGTATATTACTGTGCGAAA |
| | TGGGGGACTTATATAGTGGGAGCTACGGGTGACTACTGGGGCCAGGGAA |
| | CTCTGGTCACCGTCTCCTCA [SEQ ID NO: 20] |
| DNA for | CAGTCTGCCCTGACTCAGCCTCCCTCCGCGTCCGGGTCTCCTGGACAGT |
| Full V_{L} | CAGTCACCATCTCCTGCACTGGAACCAGCAATGACGTTGGTGGTTATAA |
| | CTATGTCTCCTGGTACCAACAGCACCCAGGCAAAGCCCCCAAACTCTTG |
| | ATTTATGAGGTCAGTAAGCGGCCCTCAGGGGTCCCTGATCGCTTCTCTG |
| | GCTCCCAGTCTGGCAACACGGCCTCCCTGACCGTCTCTGGGCTCCAGGC |
| | TGAGGATGAGGCTGATTATTACTGCAGCTCATATGCAGGCAGCAACAAT |
| | TGGGTGTTCGGCGGAGGGACCAAGCTGACCGTCCTA [SEQ ID NO: |
| | 21] |

In certain embodiments, the anti-CD33 scFv is an scFv-Fc fusion protein or full-length human IgG with V_{H} and V_{L} regions or CDRs selected from Table 3. In certain embodiments, the anti-CD33 scFv comprises a V_{H} comprising the amino acid sequence set forth in SEQ ID NO: 28, as shown in Table 3. An exemplary nucleotide sequence encoding the amino acid sequence of SEQ ID NO: 28 is set forth in SEQ ID NO: 30. In certain embodiments, the anti-CD33 scFv comprises a V_{L} comprising the amino acid sequence set forth in SEQ ID NO: 29. An exemplary nucleotide sequence encoding the amino acid sequence of SEQ ID NO: 29 is set forth in SEQ ID NO: 31. SEQ ID NO: 28-31 are provided in Table 3. In certain embodiments, the scFv is designated as “1-J19”.

In certain embodiments, the anti-CD33 scFv comprises a V_{H} comprising the amino acid sequence set forth in SEQ ID NO: 28 and a V_{L} comprising the amino acid sequence set forth in SEQ ID NO: 29.

In certain embodiments, the anti-CD33 scFv comprises a V_{H} comprising a CDR1 comprising the amino acid sequence set forth in SEQ ID NO: 22 or a conservative modification thereof, a CDR2 comprising the amino acid sequence set forth in SEQ ID NO: 23 or a conservative modification thereof, and a CDR3 comprising the amino acid sequence set forth in SEQ ID NO: 24 or a conservative modification thereof. SEQ ID NOs: 22-24 are provided in Table 3.

In certain embodiments, the anti-CD33 scFv comprises a V_{L} comprising a CDR1 comprising the amino acid sequence set forth in SEQ ID NO: 25 or a conservative modification thereof, a CDR2 comprising the amino acid sequence set forth in SEQ ID NO: 26 or a conservative modification thereof, and a CDR3 comprising the amino acid sequence set forth in SEQ ID NO: 27 or a conservative modification thereof. SEQ ID NOs: 25-27 are provided in Table 3.

In certain embodiments, the anti-CD33 scFv comprises a V_{H} comprising a CDR1 comprising the amino acid sequence set forth in SEQ ID NO: 22 or a conservative modification thereof, a CDR2 comprising the amino acid sequence set forth in SEQ ID NO: 23 or a conservative modification thereof, and a CDR3 comprising the amino acid sequence set forth in SEQ ID NO: 24 or a conservative modification thereof; and a V_{L} comprising a CDR1 comprising the amino acid sequence set forth in SEQ ID NO: 25 or a conservative modification thereof, a CDR2 comprising the amino acid sequence set forth in SEQ ID NO: 26 or a conservative modification thereof, and a CDR3 comprising the amino acid sequence set forth in SEQ ID NO: 27 or a conservative modification thereof.

In certain embodiments, the anti-CD33 scFv comprises a V_{H} comprising a CDR1 comprising the amino acid sequence set forth in SEQ ID NO: 22, a CDR2 comprising the amino acid sequence set forth in SEQ ID NO: 23, and a CDR3 comprising the amino acid sequence set forth in SEQ ID NO: 24; and a V_{L} comprising a CDR1 comprising the amino acid sequence set forth in SEQ ID NO: 25, a CDR2 comprising the amino acid sequence set forth in SEQ ID NO: 26, and a CDR3 comprising the amino acid sequence set forth in SEQ ID NO: 27.

In certain embodiments, the anti-CD33 scFv comprises a V_{H} comprising the amino acid sequence set forth in SEQ ID NO: 28, and a V_{L} comprising the amino acid sequence set forth in SEQ ID NO: 29. In certain embodiments, the V_{H} and V_{L} are linked via a linker. In certain embodiments, the linker comprises the amino acid sequence set forth in SEQ ID NO: 105.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}

TABLE 3

| CDRs | 1 | 2 | 3 |
|---|---|---|---|
| V_{H} | GDSVSSNSAA | TYFRSKWYN | ASEGGSYYDH |
| | [SEQ ID NO: 22] | [SEQ ID NO: 23] | [SEQ ID NO: 24] |
| V_{L} | QGISNW | AAS | QQADSFPFT |
| | [SEQ ID NO: 25] | [SEQ ID NO: 26] | [SEQ ID NO: 27] |

| | |
|---|---|
| Full V_{H} | QVQLQQSGPGLVKPSQTLSLTCAISGDSVSSNSAAWNWIRQSPSRGLEW |
| | LGRTYFRSKWYNVYAVSVKSRITINPDTSKNQFSLQLNSVTPEDTAVYY |
| | CASEGGSYYDHWGQGTLVTVSS [SEQ ID NO: 28] |
| Full V_{L} | DIQMTQSPSSVSASVGDRVTITCRASQGISNWLTWYQQKPGKAPKLLIY |
| | AASSLQSGVPSRFSGSGSGTDFTLTISSLQPEDFATYYCQQADSFPFTF |
| | GPGTKVDIK [SEQ ID NO: 29] |
| DNA for | CAGGTACAGCTGCAGCAGTCAGGTCCAGGACTGGTGAAGCCCTCGCAGA |
| Full V_{H} | CCCTCTCACTCACCTGTGCCATCTCCGGGGACAGTGTCTCTAGCAACAG |
| | TGCTGCTTGGAACTGGATCAGGCAGTCCCCATCGAGAGGCCTTGAGTGG |
| | CTGGGAAGGACATACTTCAGGTCCAAGTGGTATAATGTTTATGCAGTGT |
| | CTGTGAAGAGTCGAATAACCATCAACCCAGACACATCCAAGAACCAGTT |
| | CTCCCTGCAGCTGAACTCTGTGACTCCCGAGGACACGGCTGTGTATTAT |
| | TGTGCAAGCGAGGGTGGGAGCTATTATGACCACTGGGGCCAGGGAACCC |
| | TGGTCACCGTCTCCTCA [SEQ ID NO: 30] |
| DNA for | GACATCCAGATGACCCAGTCTCCATCTTCCGTGTCTGCATCTGTAGGAG |
| Full V_{L} | ACAGAGTCACCATCACTTGTCGGGCGAGTCAGGGTATTAGTAATTGGTT |
| | AACCTGGTATCAGCAGAAACCAGGGAAAGCCCCTAAGCTCCTGATCTAT |
| | GCTGCATCCAGTTTGCAAAGTGGGGTCCCATCAAGGTTCAGCGGCAGTG |
| | GATCTGGGACAGATTTCACTCTCACCATCAGCAGCCTGCAGCCTGAAGA |
| | TTTTGCAACTTACTATTGTCAACAGGCTGACAGTTTCCCATTCACTTTC |
| | GGCCCTGGGACCAAAGTGGATATCAAA [SEQ ID NO: 31] |

In certain embodiments, the anti-CD33 scFv is an scFv-Fc fusion protein or full length human IgG with V_{H} and V_{L} regions or CDRs selected from Table 4. In certain embodiments, the anti-CD33 scFv comprises a V_{H} comprising the amino acid sequence set forth in SEQ ID NO: 35, as shown in Table 4. An exemplary nucleotide sequence encoding the amino acid sequence of SEQ ID NO: 35 is set forth in SEQ ID NO: 36. In certain embodiments, the anti-CD33 scFv comprises a V_{L} comprising the amino acid sequence set forth in SEQ ID NO: 29. An exemplary nucleotide sequence encoding the amino acid sequence of SEQ ID NO: 29 is set forth in SEQ ID NO: 31. SEQ ID NO: 29, 31, 35, and 36 are provided in Table 4. In certain embodiments, the scFv is designated as “1-J19-2”

In certain embodiments, the anti-CD33 scFv comprises a V_{H} comprising the amino acid sequence set forth in SEQ ID NO: 35 and a V_{L} comprising the amino acid sequence set forth in SEQ ID NO: 29. SEQ ID NOs: 35 and 29 are provided in Table 4.

In certain embodiments, the anti-CD33 scFv comprises a V_{H} comprising a CDR1 comprising the amino acid sequence set forth in SEQ ID NO: 32 or a conservative modification thereof, a CDR2 comprising the amino acid sequence set forth in SEQ ID NO: 33 or a conservative modification thereof, and a CDR3 comprising the amino acid sequence set forth in SEQ ID NO: 34 or a conservative modification thereof. SEQ ID NOs: 32-34 are provided in Table 4.

In certain embodiments, the anti-CD33 scFv comprises a V_{L} comprising a CDR1 comprising the amino acid sequence set forth in SEQ ID NO: 25 or a conservative modification thereof, a CDR2 comprising the amino acid sequence set forth in SEQ ID NO: 26 or a conservative modification thereof, and a CDR3 comprising the amino acid sequence set forth in SEQ ID NO: 27 or a conservative modification thereof. SEQ ID NOs: 25-27 are provided in Table 4.

In certain embodiments, the anti-CD33 scFv comprises a V_{H} comprising a CDR1 comprising the amino acid sequence set forth in SEQ ID NO: 32 or a conservative modification thereof, a CDR2 comprising the amino acid sequence set forth in SEQ ID NO: 33 or a conservative modification thereof, and a CDR3 comprising the amino acid sequence set forth in SEQ ID NO: 34 or a conservative modification thereof; and a V_{L} comprising a CDR1 comprising the amino acid sequence set forth in SEQ ID NO: 25 or a conservative modification thereof, a CDR2 comprising the amino acid sequence set forth in SEQ ID NO: 26 or a conservative modification thereof, and a CDR3 comprising the amino acid sequence set forth in SEQ ID NO: 27 or a conservative modification thereof.

In certain embodiments, the anti-CD33 scFv comprises a V_{H} comprising a CDR1 comprising the amino acid sequence set forth in SEQ ID NO: 32, a CDR2 comprising the amino acid sequence set forth in SEQ ID NO: 33, and a CDR3 comprising the amino acid sequence set forth in SEQ ID NO: 34; and a V_{L} comprising a CDR1 comprising the amino acid sequence set forth in SEQ ID NO: 25, a CDR2 comprising the amino acid sequence set forth in SEQ ID NO: 26, and a CDR3 comprising the amino acid sequence set forth in SEQ ID NO: 27.

In certain embodiments, the anti-CD33 scFv comprises a V_{H} comprising the amino acid sequence set forth in SEQ ID NO: 35, and a V_{L} comprising the amino acid sequence set forth in SEQ ID NO: 29. In certain embodiments, the V_{H} and V_{L} are linked via a linker. In certain embodiments, the linker comprises the amino acid sequence set forth in SEQ ID NO: 105.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 4

| CDRs | 1 | 2 | 3 |
|---|---|---|---|
| V_{H} | GFSLSTSGMC | IDWDDDK | ARTPYSGSYNWFDP |
| | [SEQ ID NO: 32] | [SEQ ID NO: 33] | [SEQ ID NO: 34] |
| V_{L} | QGISNW | AAS | QQADSFPFT |
| | [SEQ ID NO: 25] | [SEQ ID NO: 26] | [SEQ ID NO: 27] |

| | |
|---|---|
| Full V_{H} | QVTLRESGPALVKPTQTLTLTCTFSGFSLSTSGMCVSWIRQPPGKALEW |
| | LALIDWDDDKYYSTSLKTRLTISKDTSKNQVVLTMTNMDPVDTATYYCA |
| | RTPYSGSYNWFDPWGQGTLVTVSS [SEQ ID NO: 35] |
| Full V_{L} | DIQMTQSPSSVSASVGDRVTITCRASQGISNWLTWYQQKPGKAPKLLIY |
| | AASSLQSGVPSRFSGSGSGTDFTLTISSLQPEDFATYYCQQADSFPFTE |
| | GPGTKVDIK [SEQ ID NO: 29] |
| DNA for | CAGGTCACCTTGAGGGAGTCTGGTCCTGCGCTGGTGAAACCCACACAGA |
| Full V_{H} | CCCTCACACTGACCTGCACCTTCTCTGGGTTCTCACTCAGCACTAGTGG |
| | AATGTGTGTGAGCTGGATCCGTCAGCCCCCAGGGAAGGCCCTGGAGTGG |
| | CTTGCACTCATTGATTGGGATGATGATAAATACTACAGCACATCTCTGA |
| | AGACCAGGCTCACCATCTCCAAGGACACCTCCAAAAACCAGGTGGTCCT |
| | TACAATGACCAACATGGACCCTGTGGACACAGCCACGTATTATTGTGCA |
| | CGGACCCCCTATAGTGGGAGCTACAACTGGTTCGACCCCTGGGGCCAGG |
| | GAACCCTGGTCACCGTCTCCTCA [SEQ ID NO: 36] |
| DNA for | GACATCCAGATGACCCAGTCTCCATCTTCCGTGTCTGCATCTGTAGGAG |
| Full V_{L} | ACAGAGTCACCATCACTTGTCGGGCGAGTCAGGGTATTAGTAATTGGTT |
| | AACCTGGTATCAGCAGAAACCAGGGAAAGCCCCTAAGCTCCTGATCTAT |
| | GCTGCATCCAGTTTGCAAAGTGGGGTCCCATCAAGGTTCAGCGGCAGTG |
| | GATCTGGGACAGATTTCACTCTCACCATCAGCAGCCTGCAGCCTGAAGA |
| | TTTTGCAACTTACTATTGTCAACAGGCTGACAGTTTCCCATTCACTTTC |
| | GGCCCTGGGACCAAAGTGGATATCAAA [SEQ ID NO: 31] |

In certain embodiments, the anti-CD33 scFv is an scFv-Fc fusion protein or a full-length human IgG with V_{H} and V_{L} regions or CDRs selected from Table 5. In certain embodiments, the anti-CD33 scFv comprises a V_{H} comprising the amino acid sequence set forth in SEQ ID NO: 43, as shown in Table 5. An exemplary nucleotide sequence encoding the amino acid sequence of SEQ ID NO: 43 is set forth in SEQ ID NO: 45. In certain embodiments, the anti-CD33 scFv comprises a V_{L} comprising the amino acid sequence set forth in SEQ ID NO: 44. An exemplary nucleotide sequence encoding the amino acid sequence of SEQ ID NO: 44 is set forth in SEQ ID NO: 46. SEQ ID NO: 43-46 are provided in Table 5. In certain embodiments, the scFv is designated as “1-P13”.

In certain embodiments, the anti-CD33 scFv comprises a V_{H} comprising the amino acid sequence set forth in SEQ ID NO: 43 and a V_{L} comprising the amino acid sequence set forth in SEQ ID NO: 44.

In certain embodiments, the anti-CD33 scFv comprises a V_{H} comprising a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 39 or a conservative modification thereof. SEQ ID NOs: 37-39 are provided in Table 5.

In certain embodiments, the anti-CD33 scFv comprises a V_{L} comprising a CDR1 comprising the amino acid sequence set forth in SEQ ID NO: 40 or a conservative modification thereof, a CDR2 comprising the amino acid sequence set forth in SEQ ID NO: 41 or a conservative modification thereof, and a CDR3 comprising the amino acid sequence set forth in SEQ ID NO: 42 or a conservative modification thereof. SEQ ID NOs: 40-42 are provided in Table 5.

In certain embodiments, the anti-CD33 scFv comprises a V_{H} comprising a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 39 or a conservative modification thereof; and a V_{L} comprising a CDR1 comprising the amino acid sequence set forth in SEQ ID NO: 40 or a conservative modification thereof, a CDR2 comprising the amino acid sequence set forth in SEQ ID NO: 41 or a conservative modification thereof, and a CDR3 comprising the amino acid sequence set forth in SEQ ID NO: 42 or a conservative modification thereof.

In certain embodiments, the anti-CD33 scFv comprises a V_{H} comprising a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39; and a V_{L} comprising a CDR1 comprising the amino acid sequence set forth in SEQ ID NO: 40, a CDR2 comprising the amino acid sequence set forth in SEQ ID NO: 41, and a CDR3 comprising the amino acid sequence set forth in SEQ ID NO: 42.

In certain embodiments, the anti-CD33 scFv comprises a V_{H} comprising the amino acid sequence set forth in SEQ ID NO: 43, and a V_{L} comprising the amino acid sequence set forth in SEQ ID NO: 44. In certain embodiments, the V_{H} and V_{L} are linked via a linker. In certain embodiments, the linker comprises the amino acid sequence set forth in SEQ ID NO: 105.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 5

| CDRs | 1 | 2 | 3 |
|---|---|---|---|
| V_{H} | GFTFSTYG | ISYDGSNK | ARGRVGTLDY |
| | [SEQ ID NO: 37] | [SEQ ID NO: 38] | [SEQ ID NO: 39] |
| V_{L} | RGLVYSDVNTN | KVS | MQGTHWPWT |
| | [SEQ ID NO: 40] | [SEQ ID NO: 41] | [SEQ ID NO: 42] |

| | |
|---|---|
| Full V_{H} | QVQLVESGGGVVQPGRSLRLSCAASGFTFSTYGMHWVRQAPGKGLEWVA |
| | VISYDGSNKYHGDAVKGRFTISRDNSKNTLYLQMNSLRAEDTAVYYCAR |
| | GRVGTLDYWGQGTLVTVSS [SEQ ID NO: 43] |
| Full V_{L} | DVVMTQSPLSLPVSLGQPASISCRSSRGLVYSDVNTNLNWFQQRPGQSP |
| | RRLIYKVSNRDSGVPDRFSGSGSGTDFTLKISRVEAEDVGVYYCMQGTH |
| | WPWTFGQGTKVEIK |
| | [SEQ ID NO: 44] |
| DNA for | CAGGTGCAGCTGGTGGAGTCTGGGGGAGGCGTGGTCCAGCCTGGGAGGT |
| Full V_{H} | CCCTGAGACTCTCCTGTGCAGCCTCTGGATTTACCTTCAGTACCTATGG |
| | CATGCACTGGGTCCGCCAGGCTCCAGGCAAGGGGCTGGAGTGGGTGGCA |
| | GTCATATCATATGATGGAAGTAATAAATATCATGGAGACGCCGTGAAGG |
| | GCCGGTTCACCATCTCCAGAGACAATTCCAAGAACACGCTGTATCTGCA |
| | AATGAACAGCCTGAGAGCTGAGGACACGGCTGTGTATTACTGTGCGAGG |
| | GGGAGAGTGGGAACTCTTGACTATTGGGGCCAGGGAACCCTGGTCACCG |
| | TCTCCTCA |
| | [SEQ ID NO: 45] |
| DNA for | GATGTTGTGATGACTCAGTCTCCACTCTCCCTGCCCGTCAGCCTTGGAC |
| Full V_{L} | AGCCGGCCTCCATCTCCTGCAGGTCTAGTCGAGGCCTCGTATACAGTGA |
| | TGTAAACACCAACTTGAATTGGTTTCAGCAGAGGCCAGGCCAATCTCCA |
| | AGGCGCCTAATTTATAAGGTTTCTAACCGGGACTCTGGGGTCCCAGACA |
| | GATTCAGCGGCAGTGGGTCAGGCACTGATTTCACACTGAAAATCAGCAG |
| | GGTGGAGGCTGAGGATGTTGGGGTTTATTACTGCATGCAAGGTACACAC |
| | TGGCCTTGGACGTTCGGCCAAGGGACCAAGGTGGAAATCAAA [SEQ |
| | ID NO: 46] |

In certain embodiments, the anti-DLL scFv is an scFv-Fc fusion protein or a full-length human IgG with V_{H} and V_{L} regions or CDRs selected from Table 6. In certain embodiments, the anti-CD33 scFv comprises a V_{H} comprising the amino acid sequence set forth in SEQ ID NO: 52, as shown in Table 6. An exemplary nucleotide sequence encoding the amino acid sequence of SEQ ID NO: 52 is set forth in SEQ ID NO: 54. In certain embodiments, the anti-CD33 scFv comprises a V_{L} comprising the amino acid sequence set forth in SEQ ID NO: 53. An exemplary nucleotide sequence encoding the amino acid sequence of SEQ ID NO: 53 is set forth in SEQ ID NO: 55. SEQ ID NO: 52-55 are provided in Table 6. In certain embodiments, the scFv is designated as “1-P23”.

In certain embodiments, the anti-CD33 scFv comprises a V_{H} comprising the amino acid sequence set forth in SEQ ID NO: 52 and a V_{L} comprising the amino acid sequence set forth in SEQ ID NO: 53.

In certain embodiments, the anti-CD33 scFv comprises a V_{H} comprising a CDR1 comprising the amino acid sequence set forth in SEQ ID NO: 47 or a conservative modification thereof, a CDR2 comprising the amino acid sequence set forth in SEQ ID NO: 38 or a conservative modification thereof, and a CDR3 comprising the amino acid sequence set forth in SEQ ID NO: 48 or a conservative modification thereof. SEQ ID NOs: 47, 38, and 48 are provided in Table 6.

In certain embodiments, the anti-CD33 scFv comprises a V_{L} comprising a CDR1 comprising the amino acid sequence set forth in SEQ ID NO: 49 or a conservative modification thereof, a CDR2 comprising the amino acid sequence set forth in SEQ ID NO: 50 or a conservative modification thereof, and a CDR3 comprising the amino acid sequence set forth in SEQ ID NO: 51 or a conservative modification thereof. SEQ ID NOs: 49-51 are provided in Table 6.

In certain embodiments, the anti-CD33 scFv comprises a V_{H} comprising a CDR1 comprising the amino acid sequence set forth in SEQ ID NO: 47 or a conservative modification thereof, a CDR2 comprising the amino acid sequence set forth in SEQ ID NO: 38 or a conservative modification thereof, and a CDR3 comprising the amino acid sequence set forth in SEQ ID NO: 48 or a conservative modification thereof; and a V_{L} comprising a CDR1 comprising the amino acid sequence set forth in SEQ ID NO: 49 or a conservative modification thereof, a CDR2 comprising the amino acid sequence set forth in SEQ ID NO: 50 or a conservative modification thereof, and a CDR3 comprising the amino acid sequence set forth in SEQ ID NO: 51 or a conservative modification thereof.

In certain embodiments, the anti-CD33 scFv comprises a V_{H} comprising a CDR1 comprising the amino acid sequence set forth in SEQ ID NO: 47, a CDR2 comprising the amino acid sequence set forth in SEQ ID NO: 38, and a CDR3 comprising the amino acid sequence set forth in SEQ ID NO: 48; and a V_{L} comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51.

In certain embodiments, the anti-CD33 scFv comprises a V_{H} comprising the amino acid sequence set forth in SEQ ID NO: 52, and a V_{L} comprising the amino acid sequence set forth in SEQ ID NO: 53. In certain embodiments, the V_{H} and V_{L} are linked via a linker. In certain embodiments, the linker comprises the amino acid sequence set forth in SEQ ID NO: 105.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}

TABLE 6

| CDRs | 1 | 2 | 3 |
|---|---|---|---|
| V_{H} | GFTFNSYG | ISYDGSNK | ARDNYDSSGYNWYFD |
| | [SEQ ID NO: 47] | [SEQ ID NO: 38] | L [SEQ ID NO: 48] |
| V_{L} | SLRSYY | GKN | NSRDSSGNLWV |
| | [SEQ ID NO: 49] | [SEQ ID NO: 50] | [SEQ ID NO: 51] |

| | |
|---|---|
| Full V_{H} | QVQLVESGGGVVQPGRSLRLSCAASGFTFNSYGMHWVRQAPGKGLEWVA |
| | IISYDGSNKYYADSVKGRFTISRDNSKNTLYLQMNSLRAEDTAVYYCAR |
| | DNYDSSGYNWYFDLWGRGTLVTVSS [SEQ ID NO: 52] |
| Full V_{L} | SSELTQDPAVSVALGQTVRITCQGDSLRSYYASWYQQKPGQAPVLVIYG |
| | KNNRPSGIPDRESGSSSGNTASLTITGAQAEDEADYYCNSRDSSGNLWV |
| | FGGGTKLTVL [SEQ ID NO: 53] |
| DNA for | CAGGTGCAGCTGGTGGAGTCTGGGGGAGGCGTGGTCCAGCCTGGGAGGT |
| Full V_{H} | CCCTGAGACTCTCCTGTGCAGCCTCTGGATTCACCTTCAACAGCTATGG |
| | CATGCACTGGGTCCGCCAGGCTCCAGGCAAGGGGCTGGAGTGGGTGGCA |
| | ATTATATCATATGATGGAAGTAATAAATACTATGCAGACTCCGTGAAGG |
| | GCCGATTCACCATCTCCAGAGACAATTCCAAGAACACGCTGTATCTGCA |
| | AATGAACAGCCTGAGAGCTGAGGACACGGCTGTGTATTATTGTGCGAGA |
| | GATAATTATGATAGTAGTGGTTATAACTGGTACTTCGATCTCTGGGGCC |
| | GTGGCACCCTGGTCACTGTCTCCTCA [SEQ ID NO: 54] |
| DNA for | TCTTCTGAGCTGACTCAGGACCCTGCTGTGTCTGTGGCCTTGGGACAGA |
| Full V_{L} | CAGTCAGGATCACATGCCAAGGAGACAGCCTCAGAAGCTATTATGCAAG |
| | CTGGTACCAGCAGAAGCCAGGACAGGCCCCTGTACTTGTCATCTATGGT |
| | AAAAACAACCGGCCCTCAGGGATCCCAGACCGATTCTCTGGCTCCAGCT |
| | CAGGAAACACAGCTTCCTTGACCATCACTGGGGCTCAGGCGGAAGATGA |
| | GGCTGACTATTACTGTAACTCCCGGGACAGCAGTGGTAACCTCTGGGTG |
| | TTCGGCGGAGGGACCAAGCTGACCGTCCTA [SEQ ID NO: 55] |

In certain embodiments, the anti-CD33 scFv is an scFv-Fc fusion protein or a full-length human IgG with V_{H} and V_{L} regions or CDRs selected from Table 7. In certain embodiments, the anti-CD33 scFv comprises a V_{H} comprising the amino acid sequence set forth in SEQ ID NO: 62, as shown in Table 7. An exemplary nucleotide sequence encoding the amino acid sequence of SEQ ID NO: 62 is set forth in SEQ ID NO: 64. In certain embodiments, the anti-CD33 scFv comprises a V_{L} comprising the amino acid sequence set forth in SEQ ID NO: 63. An exemplary nucleotide sequence encoding the amino acid sequence of SEQ ID NO: 63 is set forth in SEQ ID NO: 65. SEQ ID NO: 62-65 are provided in Table 7. In certain embodiments, the scFv is designated as “1-A20”.

In certain embodiments, the anti-CD33 scFv comprises a V_{H} comprising the amino acid sequence set forth in SEQ ID NO: 62 and a V_{L} comprising the amino acid sequence set forth in SEQ ID NO: 63.

In certain embodiments, the anti-CD33 scFv comprises a V_{H} comprising a CDR1 comprising the amino acid sequence set forth in SEQ ID NO: 56 or a conservative modification thereof, a CDR2 comprising the amino acid sequence set forth in SEQ ID NO: 57 or a conservative modification thereof, and a CDR3 comprising the amino acid sequence set forth in SEQ ID NO: 58 or a conservative modification thereof. SEQ ID NOs: 56-58 are provided in Table 7.

In certain embodiments, the anti-CD33 scFv comprises a V_{L} comprising a CDR1 comprising the amino acid sequence set forth in SEQ ID NO: 59 or a conservative modification thereof, a CDR2 comprising the amino acid sequence set forth in SEQ ID NO: 60 or a conservative modification thereof, and a CDR3 comprising the amino acid sequence set forth in SEQ ID NO: 61 or a conservative modification thereof. SEQ ID NOs: 59-61 are provided in Table 7.

In certain embodiments, the anti-CD33 scFv comprises a V_{H} comprising a CDR1 comprising the amino acid sequence set forth in SEQ ID NO: 56 or a conservative modification thereof, a CDR2 comprising the amino acid sequence set forth in SEQ ID NO: 57 or a conservative modification thereof, and a CDR3 comprising the amino acid sequence set forth in SEQ ID NO: 58 or a conservative modification thereof; and a V_{L} comprising a CDR1 comprising the amino acid sequence set forth in SEQ ID NO: 59 or a conservative modification thereof, a CDR2 comprising the amino acid sequence set forth in SEQ ID NO: 60 or a conservative modification thereof, and a CDR3 comprising the amino acid sequence set forth in SEQ ID NO: 61 or a conservative modification thereof.

In certain embodiments, the anti-CD33 scFv comprises a V_{H} comprising a CDR1 comprising the amino acid sequence set forth in SEQ ID NO: 56, a CDR2 comprising the amino acid sequence set forth in SEQ ID NO: 57, and a CDR3 comprising the amino acid sequence set forth in SEQ ID NO: 58; and a V_{L} comprising a CDR1 comprising the amino acid sequence set forth in SEQ ID NO: 59, a CDR2 comprising the amino acid sequence set forth in SEQ ID NO: 60, and a CDR3 comprising the amino acid sequence set forth in SEQ ID NO: 61.

In certain embodiments, the anti-CD33 scFv comprises a V_{H} comprising the amino acid sequence set forth in SEQ ID NO: 62 and a V_{L} comprising the amino acid sequence set forth in SEQ ID NO: 63. In certain embodiments, the V_{H} and V_{L} are linked via a linker. In certain embodiments, the linker comprises the amino acid sequence set forth in SEQ ID NO: 105.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 7

| CDRs | 1 | 2 | 3 |
|---|---|---|---|
| V_{H} | GDSISSYY | IYTSGNT | ARDGDNRDSDAFDI |
| | [SEQ ID NO: 56] | [SEQ ID NO: 57] | [SEQ ID NO: 58] |
| V_{L} | QNISSSY | GTS | QQYGSSPLT |
| | [SEQ ID NO: 59] | [SEQ ID NO: 60] | [SEQ ID NO: 61] |

| | |
|---|---|
| Full V_{H} | QVQLQESGPGLVKPSETLSLTCTVSGDSISSYYWSWIRQPAGKGLEWIG |
| | RIYTSGNTNYNPSLKSRVTMSVDKSKNQFSLKLRSVTAADTAVYYCARD |
| | GDNRDSDAFDIWGQGTMVTVSS [SEQ ID NO: 62] |
| Full V_{L} | SPGTLSLSPGERATLSCRASQNISSSYLAWCQQKPGQAPRLFIYGTSRR |
| | ATGIPDRFSGSGSGTDETLTISRLEPEDFAVYYCQQYGSSPLTFGGGTK |
| | VEIK [SEQ ID NO: 63] |
| DNA for | CAGGTGCAGCTGCAGGAGTCGGGCCCAGGACTGGTGAAGCCTTCGGAGA |
| Full V_{H} | CCCTGTCCCTCACCTGCACTGTCTCTGGTGACTCCATCAGTAGTTATTA |
| | CTGGAGCTGGATCCGGCAGCCCGCCGGGAAGGGACTGGAGTGGATTGGA |
| | CGTATCTATACCAGTGGGAACACCAACTACAACCCCTCCCTCAAGAGTC |
| | GAGTCACCATGTCAGTAGACAAGTCCAAGAACCAGTTCTCCCTGAAGCT |
| | GAGGTCTGTGACCGCCGCGGACACGGCCGTGTATTACTGTGCGAGAGAT |
| | GGTGATAACCGGGACTCTGATGCTTTTGATATCTGGGGCCAAGGGACAA |
| | TGGTCACCGTCTCTTCA [SEQ ID NO: 64] |
| DNA for | GAAATTGTGTTGACGCAGTCTCCAGGCACCCTGTCTTTGTCTCCAGGGG |
| Full V_{L} | AAAGAGCCACCCTCTCCTGCAGGGCCAGTCAGAATATTAGCAGCAGCTA |
| | CTTAGCCTGGTGCCAGCAGAAACCTGGCCAGGCTCCCAGGCTCTTCATC |
| | TATGGTACATCCCGCAGGGCCACTGGCATCCCAGACAGGTTCAGTGGCA |
| | GTGGGTCTGGGACAGACTTCACTCTCACCATCAGCAGACTGGAGCCTGA |
| | AGATTTTGCAGTGTATTACTGTCAGCAGTATGGTAGTTCACCTCTCACT |
| | TTCGGCGGAGGGACCAAGGTGGAGATCAAA [SEQ ID NO: 65] |

In certain embodiments, the anti-CD33 scFv is an scFv-Fc fusion protein or a full-length human IgG with V_{H} and V_{L} regions or CDRs selected from Table 8. In certain embodiments, the anti-CD33 scFv comprises a V_{H} comprising the amino acid sequence set forth in SEQ ID NO: 72, as shown in Table 8. An exemplary nucleotide sequence encoding the amino acid sequence of SEQ ID NO: 72 is set forth in SEQ ID NO: 74. In certain embodiments, the anti-CD33 scFv comprises a V_{L} comprising the amino acid sequence set forth in SEQ ID NO: 73. An exemplary nucleotide sequence encoding the amino acid sequence of SEQ ID NO: 73 is set forth in SEQ ID NO: 75. SEQ ID NO: 72-75 are provided in Table 8. In certain embodiments, the scFv is designated as “2-N3”.

In certain embodiments, the anti-CD33 scFv comprises a V_{H} comprising the amino acid sequence set forth in SEQ ID NO: 72 and a V_{L} comprising the amino acid sequence set forth in SEQ ID NO: 73.

In certain embodiments, the anti-CD33 scFv comprises a V_{H} comprising a CDR1 comprising the amino acid sequence set forth in SEQ ID NO: 66 or a conservative modification thereof, a CDR2 comprising the amino acid sequence set forth in SEQ ID NO: 67 or a conservative modification thereof, and a CDR3 comprising the amino acid sequence set forth in SEQ ID NO: 68 or a conservative modification thereof. SEQ ID NOs: 66-68 are provided in Table 8.

In certain embodiments, the anti-CD33 scFv comprises a V_{L} comprising a CDR1 comprising the amino acid sequence set forth in SEQ ID NO: 69 or a conservative modification thereof, a CDR2 comprising the amino acid sequence set forth in SEQ ID NO: 70 or a conservative modification thereof, and a CDR3 comprising the amino acid sequence set forth in SEQ ID NO: 71 or a conservative modification thereof. SEQ ID NOs: 69-71 are provided in Table 8.

In certain embodiments, the anti-CD33 scFv comprises a V_{H} comprising a CDR1 comprising the amino acid sequence set forth in SEQ ID NO: 66 or a conservative modification thereof, a CDR2 comprising the amino acid sequence set forth in SEQ ID NO: 67 or a conservative modification thereof, a CDR3 comprising the amino acid sequence set forth in SEQ ID NO: 68 or a conservative modification thereof; and a V_{L} comprising a CDR1 comprising the amino acid sequence set forth in SEQ ID NO: 69 or a conservative modification thereof, a CDR2 comprising the amino acid sequence set forth in SEQ ID NO: 70 or a conservative modification thereof, and a CDR3 comprising the amino acid sequence set forth in SEQ ID NO: 71 or a conservative modification thereof.

In certain embodiments, the anti-CD33 scFv comprises a V_{H} comprising a CDR1 comprising the amino acid sequence set forth in SEQ ID NO: 66, a CDR2 comprising the amino acid sequence set forth in SEQ ID NO: 67, a CDR3 comprising the amino acid sequence set forth in SEQ ID NO: 68; and a V_{L} comprising a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71.

In certain embodiments, the anti-CD33 scFv comprises a V_{H} comprising the amino acid sequence set forth in SEQ ID NO: 72, and a V_{L} comprising the amino acid sequence set forth in SEQ ID NO: 73. In certain embodiments, the V_{H} and V_{L} are linked via a linker. In certain embodiments, the linker comprises the amino acid sequence set forth in SEQ ID NO: 105.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 8

| CDRs | 1 | 2 | 3 |
|---|---|---|---|
| V_{H} | GFSFTSHW | IYPGDSDT | ARHETGNGYSYGMDV |
| | [SEQ ID NO: 66] | [SEQ ID NO: 67] | [SEQ ID NO: 68] |
| V_{L} | QSLLHSNGYNY | LGS | MQALQPPLT |
| | [SEQ ID NO: 69] | [SEQ ID NO: 70] | [SEQ ID NO: 71] |

| | |
|---|---|
| Full V_{H} | EVQLVQSGADVKKPGESLKISCKGSGFSFTSHWIAWVRQMPGKGLEWMG |
| | IIYPGDSDTRYSPSLQGRVTISADKSISTAYLQWSSLKASDTAMYFCAR |
| | HETGNGYSYGMDVWGQGTTVTVSS [SEQ ID NO: 72] |
| Full V_{L} | DIVMTQSPLSLPVTPGEPASISCRSSQSLLHSNGYNYLDWYLQKPGQSP |
| | QLLFSLGSNRASGVPDRFSGSGSGTDFSLKISRVEAEDVGLYYCMQALQ |
| | PPLTFGGGTKVEIK [SEQ ID NO: 73] |
| DNA for | GAGGTGCAGCTGGTGCAGTCTGGAGCAGACGTGAAAAAGCCCGGGGAGT |
| Full V_{H} | CTCTGAAGATCTCCTGTAAGGGTTCTGGATTCAGTTTTACCAGCCACTG |
| | GATCGCCTGGGTGCGCCAGATGCCCGGGAAAGGCCTGGAGTGGATGGGG |
| | ATAATCTATCCTGGTGACTCTGATACCAGATACAGCCCGTCCTTACAAG |
| | GCCGGGTCACCATCTCAGCCGACAAGTCCATCAGCACCGCCTACCTGCA |
| | GTGGAGCAGCCTGAAGGCCTCGGACACCGCCATGTATTTCTGTGCGAGG |
| | CATGAAACTGGGAATGGCTACTCCTACGGAATGGACGTCTGGGGCCAAG |
| | GGACCACGGTCACCGTCTCCTCA [SEQ ID NO: 74] |
| DNA for | GATATTGTGATGACTCAGTCTCCACTCTCCCTGCCCGTCACCCCTGGAG |
| Full V_{L} | AGCCGGCCTCCATCTCCTGCAGGTCTAGTCAGAGCCTCCTGCATAGTAA |
| | TGGATACAACTATTTGGATTGGTACCTGCAGAAGCCAGGGCAGTCTCCA |
| | CAGCTCCTGTTCTCTTTGGGTTCTAATCGGGCCTCCGGGGTCCCTGACA |
| | GGTTCAGTGGCAGTGGATCAGGCACAGATTTTTCACTGAAAATCAGCAG |
| | AGTGGAGGCTGAGGATGTTGGGCTTTATTACTGCATGCAAGCTCTACAA |
| | CCTCCGCTCACTTTCGGCGGAGGGACCAAGGTGGAGATCAAA [SEQ |
| | ID NO: 75] |

In certain embodiments, the anti-CD33 scFv is an scFv-Fc fusion protein or a full-length human IgG with V_{H} and V_{L} regions or CDRs selected from Table 9. In certain embodiments, the anti-CD33 scFv comprises a V_{H} comprising the amino acid sequence set forth in SEQ ID NO: 81, as shown in Table 9. An exemplary nucleotide sequence encoding the amino acid sequence of SEQ ID NO: 81 is set forth in SEQ ID NO: 83. In certain embodiments, the anti-CD33 scFv comprises a V_{L} comprising the amino acid sequence set forth in SEQ ID NO: 82. An exemplary nucleotide sequence encoding the amino acid sequence of SEQ ID NO: 82 is set forth in SEQ ID NO: 84. SEQ ID NO: 81-84 are provided in Table 9. In certain embodiments, the scFv is designated as “1-H19”.

In certain embodiments, the anti-CD33 scFv comprises a V_{H} comprising the amino acid sequence set forth in SEQ ID NO: 81 and a V_{L} comprising the amino acid sequence set forth in SEQ ID NO: 82.

In certain embodiments, the anti-CD33 scFv comprises a V_{H} comprising a CDR1 comprising the amino acid sequence set forth in SEQ ID NO: 76 or a conservative modification thereof, a CDR2 comprising the amino acid sequence set forth in SEQ ID NO: 57 or a conservative modification thereof, and a CDR3 comprising the amino acid sequence set forth in SEQ ID NO: 77 or a conservative modification thereof. SEQ ID NOs: 76, 57, and 77 are provided in Table 9.

In certain embodiments, the anti-CD33 scFv comprises a V_{L} comprising a CDR1 comprising the amino acid sequence set forth in SEQ ID NO: 78 or a conservative modification thereof, a CDR2 comprising the amino acid sequence set forth in SEQ ID NO: 79 or a conservative modification thereof, and a CDR3 comprising the amino acid sequence set forth in SEQ ID NO: 80 or a conservative modification thereof. SEQ ID NOs: 78-80 are provided in Table 9.

In certain embodiments, the anti-CD33 scFv comprises a V_{H} comprising a CDR1 comprising the amino acid sequence set forth in SEQ ID NO: 76 or a conservative modification thereof, a CDR2 comprising the amino acid sequence set forth in SEQ ID NO: 57 or a conservative modification thereof, and a CDR3 comprising the amino acid sequence set forth in SEQ ID NO: 77 or a conservative modification thereof; and a V_{L} comprising a CDR1 comprising the amino acid sequence set forth in SEQ ID NO: 78 or a conservative modification thereof, a CDR2 comprising the amino acid sequence set forth in SEQ ID NO: 79 or a conservative modification thereof, and a CDR3 comprising the amino acid sequence set forth in SEQ ID NO: 80 or a conservative modification thereof.

In certain embodiments, the anti-CD33 scFv comprises a V_{H} comprising a CDR1 comprising the amino acid sequence set forth in SEQ ID NO: 76, a CDR2 comprising the amino acid sequence set forth in SEQ ID NO: 57, and a CDR3 comprising the amino acid sequence set forth in SEQ ID NO: 77; and a V_{L} comprising a CDR1 comprising the amino acid sequence set forth in SEQ ID NO: 78, a CDR2 comprising the amino acid sequence set forth in SEQ ID NO: 79, and a CDR3 comprising the amino acid sequence set forth in SEQ ID NO: 80.

In certain embodiments, the anti-CD33 scFv comprises a V_{H} comprising the amino acid sequence set forth in SEQ ID NO: 81, and a V_{L} comprising the amino acid sequence set forth in SEQ ID NO: 82. In certain embodiments, the V_{H} and V_{L} are linked via a linker. In certain embodiments, the linker comprises the amino acid sequence set forth in SEQ ID NO: 105.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 9

| CDRs | 1 | 2 | 3 |
|---|---|---|---|
| V_{H} | GGSISTYY | IYTSGNT | ARDGDNRNSDAFDI |
| | [SEQ ID NO: 76] | [SEQ ID NO: 57] | [SEQ ID NO: 77] |
| V_{L} | SSNIGAGYD | GNS | QSYDRSLSGWV |
| | [SEQ ID NO: 78] | [SEQ ID NO: 79] | [SEQ ID NO: 80] |

| | |
|---|---|
| Full V_{H} | QVQLQESGPGLVKPSETLSLTCTVSGGSISTYYWSWIRQPAGKGLEWIG |
| | RIYTSGNTNYNPSLKSRVTMSVDTSKNQLSLKLRSVTAADTAVYYCARD |
| | GDNRNSDAFDIWGQGTMVTVSS [SEQ ID NO: 81] |
| Full V_{L} | QSVLTQPPSVSGAPGORVTISCTGSSSNIGAGYDVHWYQQLPGTAPKLL |
| | IYGNSNRPSGVPDRESGSKSGTSASLAITGLQAEDEADYYCQSYDRSLS |
| | GWVFGGGTKLTVL [SEQ ID NO: 82] |
| DNA for | CAGGTGCAGCTGCAGGAGTCGGGCCCAGGACTGGTGAAGCCTTCGGAGA |
| Full V_{H} | CCCTGTCCCTCACCTGCACTGTCTCTGGTGGCTCCATCAGTACTTATTA |
| | CTGGAGCTGGATCCGGCAGCCCGCCGGGAAGGGACTGGAGTGGATTGGG |
| | CGTATCTATACCAGTGGGAACACCAACTACAACCCCTCCCTCAAGAGTC |
| | GAGTCACCATGTCAGTAGACACGTCCAAGAATCAGCTCTCCCTGAAGCT |
| | GAGGTCTGTGACCGCCGCGGACACGGCCGTGTATTACTGTGCGAGAGAT |
| | GGGGATAACCGGAACTCTGATGCTTTTGATATTTGGGGCCAAGGGACAA |
| | TGGTCACCGTCTCTTCA [SEQ ID NO: 83] |
| DNA for | CAGTCTGTGCTGACGCAGCCGCCCTCAGTGTCTGGGGCCCCAGGGCAGA |
| Full V_{L} | GGGTCACCATCTCCTGCACTGGGAGCAGCTCCAACATCGGGGCAGGTTA |
| | TGATGTACACTGGTACCAGCAGCTTCCAGGAACAGCCCCCAAACTCCTC |
| | ATCTATGGTAACAGCAATCGGCCCTCAGGGGTCCCTGACCGATTCTCTG |
| | GCTCCAAGTCTGGCACCTCAGCCTCCCTGGCCATCACTGGGCTCCAGGC |
| | TGAGGATGAGGCTGATTATTACTGCCAGTCCTATGACCGCAGCCTGAGT |
| | GGTTGGGTGTTCGGCGGAGGGACCAAGCTGACCGTCCTA [SEQ ID |
| | NO: 84] |

In certain embodiments, the anti-CD33 scFv is an scFv-Fc fusion protein or a full-length human IgG with V_{H} and V_{L} regions or CDRs selected from Table 10. In certain embodiments, the anti-CD33 scFv comprises a V_{H} comprising the amino acid sequence set forth in SEQ ID NO: 88, as shown in Table 10. An exemplary nucleotide sequence encoding the amino acid sequence of SEQ ID NO: 88 is set forth in SEQ ID NO: 90. In certain embodiments, the anti-CD33 scFv comprises a V_{L} comprising the amino acid sequence set forth in SEQ ID NO: 89. An exemplary nucleotide sequence encoding the amino acid sequence of SEQ ID NO: 89 is set forth in SEQ ID NO: 91. SEQ ID NO: 88-91 are provided in Table 10. In certain embodiments, the scFv is designated as “2-F18”.

In certain embodiments, the anti-CD33 scFv comprises a V_{H} comprising the amino acid sequence set forth in SEQ ID NO: 88 and a V_{L} comprising the amino acid sequence set forth in SEQ ID NO: 89.

In certain embodiments, the anti-CD33 scFv comprises a V_{H} comprising a CDR1 comprising the amino acid sequence set forth in SEQ ID NO: 85 or a conservative modification thereof, a CDR2 comprising the amino acid sequence set forth in SEQ ID NO: 86 or a conservative modification thereof, and a CDR3 comprising the amino acid sequence set forth in SEQ ID NO: 87 or a conservative modification thereof. SEQ ID NOs: 85-87 are provided in Table 10.

In certain embodiments, the anti-CD33 scFv comprises a V_{L} comprising a CDR1 comprising the amino acid sequence set forth in SEQ ID NO: 5 or a conservative modification thereof, a CDR2 comprising the amino acid sequence set forth in SEQ ID NO: 41 or a conservative modification thereof, and a CDR3 comprising the amino acid sequence set forth in SEQ ID NO: 42 or a conservative modification thereof. SEQ ID NOs: 5, 41, and 42 are provided in Table 10.

In certain embodiments, the anti-CD33 scFv comprises a V_{H} comprising a CDR1 comprising the amino acid sequence set forth in SEQ ID NO: 85 or a conservative modification thereof, a CDR2 comprising the amino acid sequence set forth in SEQ ID NO: 86 or a conservative modification thereof, and a CDR3 comprising the amino acid sequence set forth in SEQ ID NO: 87 or a conservative modification thereof; and a V_{L} comprising a CDR1 comprising the amino acid sequence set forth in SEQ ID NO: 5 or a conservative modification thereof, a CDR2 comprising the amino acid sequence set forth in SEQ ID NO: 41 or a conservative modification thereof, and a CDR3 comprising the amino acid sequence set forth in SEQ ID NO: 42 or a conservative modification thereof.

In certain embodiments, the anti-CD33 scFv comprises a V_{H} comprising a CDR1 comprising the amino acid sequence set forth in SEQ ID NO: 85, a CDR2 comprising the amino acid sequence set forth in SEQ ID NO: 86, and a CDR3 comprising the amino acid sequence set forth in SEQ ID NO: 87; and a V_{L} comprising a CDR1 comprising the amino acid sequence set forth in SEQ ID NO: 5, a CDR2 comprising the amino acid sequence set forth in SEQ ID NO: 41, and a CDR3 comprising the amino acid sequence set forth in SEQ ID NO: 42.

In certain embodiments, the anti-CD33 scFv comprises a V_{H} comprising the amino acid sequence set forth in SEQ ID NO: 88, and a V_{L} comprising the amino acid sequence set forth in SEQ ID NO: 89. In certain embodiments, the V_{H} and V_{L} are linked via a linker. In certain embodiments, the linker comprises the amino acid sequence set forth in SEQ ID NO: 105.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 10

| CDRs | 1 | 2 | 3 |
|---|---|---|---|
| V_{H} | GFTFSAYP | ISYDGSNN | ARGKVGTLDF |
| | [SEQ ID NO: 85] | [SEQ ID NO: 86] | [SEQ ID NO: 87] |
| V_{L} | QSLVYSDGNTY | KVS | MQGTHWPWT |
| | [SEQ ID NO: 5] | [SEQ ID NO: 41] | [SEQ ID NO: 42] |

| | |
|---|---|
| Full V_{H} | QIQLVESGGGVVQPGRSLRLSCAASGFTFSAYPMHWVRQAPGKGLEWVA |
| | IISYDGSNNYYADSVKGRFTISRDNSKNTMYLQINSLRAEDTGVYYCAR |
| | GKVGTLDFWGQGTLVTVSS [SEQ ID NO: 88] |
| Full V_{L} | DVVLTQSPLSLPVTLGQPASISCRSSQSLVYSDGNTYLNWFQQRPGQSP |
| | RRLIYKVSNRDSGVPDRFSGSGSGTDFTLKISRVEAEDVGVYYCMQGTH |
| | WPWTFGQGTKVEIK [SEQ ID NO: 89] |
| DNA for | CAGATACAGCTGGTGGAGTCTGGGGGAGGCGTGGTCCAGCCTGGGAGGT |
| Full V_{H} | CCCTGAGACTCTCCTGTGCAGCCTCTGGATTCACCTTCAGTGCCTATCC |
| | CATGCACTGGGTCCGCCAGGCTCCAGGCAAGGGGCTGGAGTGGGTGGCA |
| | ATTATATCATATGATGGAAGTAATAACTACTATGCAGACTCCGTGAAGG |
| | GCCGATTCACCATCTCCAGAGACAATTCCAAGAACACGATGTATCTGCA |
| | AATTAACAGCCTGAGAGCTGAGGACACGGGTGTGTATTACTGTGCGCGA |
| | GGGAAAGTGGGAACCCTTGACTTCTGGGGCCAGGGAACCCTGGTCACCG |
| | TCTCCTCA [SEQ ID NO: 90] |
| DNA for | GATGTTGTGCTGACTCAGTCTCCACTCTCCCTGCCCGTCACCCTTGGAC |
| Full V_{L} | AGCCGGCCTCCATCTCCTGCAGGTCTAGTCAAAGCCTCGTATACAGTGA |
| | TGGAAACACCTACTTGAATTGGTTTCAGCAGAGGCCAGGCCAATCTCCA |
| | AGGCGCCTAATTTATAAGGTTTCTAACCGGGACTCTGGGGTCCCAGACA |
| | GATTCAGCGGCAGTGGGTCAGGCACTGATTTCACACTGAAAATCAGCAG |
| | GGTGGAGGCTGAGGATGTTGGGGTTTATTACTGCATGCAAGGTACACAC |
| | TGGCCGTGGACGTTCGGCCAAGGGACCAAGGTGGAAATCAAA [SEQ |
| | ID NO: 91] |

In certain embodiments, the anti-CD33 scFv is an scFv-Fc fusion protein or a full-length human IgG with V_{H} and V_{L} regions or CDRs selected from Table 11. In certain embodiments, the anti-CD33 scFv comprises a V_{H} comprising the amino acid sequence set forth in SEQ ID NO: 8, as shown in Table 11. An exemplary nucleotide sequence encoding the amino acid sequence of SEQ ID NO: 8 is set forth in SEQ ID NO: 93. In certain embodiments, the anti-CD33 scFv comprises a V_{L} comprising the amino acid sequence set forth in SEQ ID NO: 92. An exemplary nucleotide sequence encoding the amino acid sequence of SEQ ID NO: 92 is set forth in SEQ ID NO: 94. SEQ ID NO: 8, 92, 93, and 94 are provided in Table 11. In certain embodiments, the scFv is designated as “4-P3”.

In certain embodiments, the anti-CD33 scFv comprises a V_{H} comprising the amino acid sequence set forth in SEQ ID NO: 8 and a V_{L} comprising the amino acid sequence set forth in SEQ ID NO: 92.

In certain embodiments, the anti-CD33 scFv comprises a V_{H} comprising a CDR1 comprising the amino acid sequence set forth in SEQ ID NO: 2 or a conservative modification thereof, a CDR2 comprising the amino acid sequence set forth in SEQ ID NO: 3 or a conservative modification thereof, and a H CDR3 comprising the amino acid sequence set forth in SEQ ID NO: 4 or a conservative modification thereof. SEQ ID NOs: 2-4 are provided in Table 11.

In certain embodiments, the anti-DDL3 scFv comprises a V_{L} comprising a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 7 or a conservative modification thereof. SEQ ID NOs: 5-7 are provided in Table 11.

In certain embodiments, the anti-CD33 scFv comprises a V_{H} comprising a CDR1 comprising the amino acid sequence set forth in SEQ ID NO: 2 or a conservative modification thereof, a CDR2 comprising the amino acid sequence set forth in SEQ ID NO: 3 or a conservative modification thereof, and a CDR3 comprising the amino acid sequence set forth in SEQ ID NO: 4 or a conservative modification thereof; and a V_{L} comprising a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 7 or a conservative modification thereof.

In certain embodiments, the anti-CD33 scFv comprises a V_{H} comprising a CDR1 comprising the amino acid sequence set forth in SEQ ID NO: 2, a CDR2 comprising the amino acid sequence set forth in SEQ ID NO: 3, and a CDR3 comprising the amino acid sequence set forth in SEQ ID NO: 4, a V_{L} comprising a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7.

In certain embodiments, the anti-CD33 scFv comprises a V_{H} comprising the amino acid sequence set forth in SEQ ID NO: 8, and a V_{L} comprising the amino acid sequence set forth in SEQ ID NO: 92. In certain embodiments, the V_{H} and V_{L} are linked via a linker. In certain embodiments, the linker comprises the amino acid sequence set forth in SEQ ID NO: 105.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 11

| CDRs | 1 | 2 | 3 |
|---|---|---|---|
| V_{H} | GFTFSTYA | ISGRGGST | AGRGDYYYYYGMDV |
| | [SEQ ID NO: 2] | [SEQ ID NO: 3] | [SEQ ID NO: 4] |
| V_{L} | QSLVYSDGNTY | KIS | MQSTQFPHT |
| | [SEQ ID NO: 5] | [SEQ ID NO: 6] | [SEQ ID NO: 7] |

| | |
|---|---|
| Full V_{H} | EVQLLESGGGLVQPGGSLRLSCAASGFTFSTYAMSWVRQAPGKGLEWVS |
| | AISGRGGSTYYTDSVKGRFTISRDNSKNTVSLQMNSLRAEDTAVYYCAG |
| | RGDYYYYYGMDVWGQGTTVTVSA [SEQ ID NO: 8] |
| Full V_{L} | DIVMTQSPLSSPVTLGQPASISCRSSQSLVYSDGNTYLSWLQQRPGQPP |
| | RLLIYKISNRFSGVPDRFSGSGAGTDFTLKISRVEAEDVGVYYCMQSTQ |
| | FPHTFGQGTKLEIK [SEQ ID NO: 92] |
| DNA for | GAGGTGCAGCTGTTGGAGTCTGGGGGAGGCTTGGTCCAGCCTGGGGGGT |
| Full V_{H} | CCCTGAGACTCTCCTGTGCAGCCTCTGGATTCACCTTTAGCACCTATGC |
| | CATGAGCTGGGTCCGCCAGGCTCCAGGGAAGGGGCTGGAGTGGGTCTCA |
| | GCTATTAGTGGTCGTGGTGGTAGCACATACTACACAGACTCCGTGAAGG |
| | GCCGGTTCACCATCTCCAGAGACAATTCCAAGAATACGGTGTCTCTGCA |
| | AATGAACAGCCTGAGAGCCGAGGACACGGCCGTATATTACTGTGCGGGC |
| | CGGGGAGATTACTACTACTACTACGGTATGGACGTCTGGGGCCAAGGGA |
| | CCACGGTCACCGTCTCCGCA [SEQ ID NO: 93] |
| DNA for | GATATTGTGATGACCCAGAGTCCACTCTCCTCACCTGTCACCCTTGGAC |
| Full V_{L} | AGCCGGCCTCCATCTCCTGCAGGTCTAGTCAAAGCCTCGTATACAGTGA |
| | TGGAAACACCTACTTGAGTTGGCTTCAGCAGAGGCCAGGCCAGCCTCCA |
| | AGACTCCTAATTTATAAGATTTCTAACCGGTTCTCTGGGGTCCCAGACA |
| | GATTCAGTGGCAGTGGGGCAGGGACAGATTTCACACTGAAAATCAGCAG |
| | GGTGGAAGCTGAGGATGTCGGGGTTTATTACTGCATGCAATCTACACAA |
| | TTTCCTCACACTTTTGGCCAGGGGACCAAGCTGGAGATCAAA [SEQ |
| | ID NO: 94] |

### 5.3.2. Monoclonal Antibodies

The presently disclosed subject matter provides antibodies (e.g., human antibodies, e.g., human monoclonal antibodies) that specifically bind to CD33 (e.g., human CD33). The V_{H} amino acid sequences of anti-CD33 antibodies 3-P14, 4-B2, 1-J19, 1-J19-2, 1-P13, 1-P23, 1-A20, 2-N3, 1-H19, 2-F18, and 4-P3 are set forth in SEQ ID NOs: 8, 18, 28, 35, 43, 52, 62, 72, 81, and 88. The V_{L} amino acid sequences of 3-P14, 4-B2, 1-J19, 1-J19-2, 1-P13, 1-P23, 1-A20, 2-N3, 1-H19, 2-F18, and 4-P3 are set forth in SEQ ID NOs: 9, 19, 29, 44, 53, 63, 73, 82, 89, and 92.

Given that each of 3-P14, 4-B2, 1-J19, 1-J19-2, 1-P13, 1-P23, 1-A20, 2-N3, 1-H19, 2-F18, and 4-P3 antibodies can bind to CD33, the V_{H} and V_{L} sequences can be “mixed and matched” to create other anti-CD33 binding molecules. CD33 binding of such “mixed and matched” antibodies can be tested using the binding assays known in the art, including for example, ELISAs, Western blots, RIAs, Biacore analysis. Preferably, when V_{H} and V_{L} chains are mixed and matched, a V_{H} sequence from a particular V_{H}/V_{L} pairing is replaced with a structurally similar V_{H} sequence. Likewise, a V_{L} sequence from a particular V_{H}/V_{L} pairing is replaced with a structurally similar V_{L} sequence.

In certain embodiments, the presently disclosed subject matter provides an antibody or an antigen-binding fragment thereof comprising: (a) a heavy chain variable region (V_{H}) comprising an amino acid sequence selected from SEQ ID NOs: 8, 18, 28, 35, 43, 52, 62, 72, 81, and 88; and (b) a light chain variable region (V_{L}) comprising an amino acid sequence selected from SEQ ID NOs: 9, 19, 29, 44, 53, 63, 73, 82, 89, and 92; wherein the antibody or antigen-binding fragment specifically binds to CD33, e.g., human CD33. In certain embodiments, the V_{H} and V_{L} are selected from the group consisting of:
(a) a heavy chain variable region comprising the amino acid sequence set forth in SEQ ID NO: 8, and a light chain variable region comprising the amino acid sequence set forth in SEQ ID NO: 9;
(b) a heavy chain variable region comprising the amino acid sequence set forth in SEQ ID NO: 18, and a light chain variable region comprising the amino acid sequence set forth in SEQ ID NO: 19;
(c) a heavy chain variable region comprising the amino acid sequence set forth in SEQ ID NO: 28, and a light chain variable region comprising the amino acid sequence set forth in SEQ ID NO: 29;
(d) a heavy chain variable region comprising the amino acid sequence set forth in SEQ ID NO: 35, and a light chain variable region comprising the amino acid sequence set forth in SEQ ID NO: 29;
(e) a heavy chain variable region comprising the amino acid sequence set forth in SEQ ID NO: 43, and a light chain variable region comprising the amino acid sequence set forth in SEQ ID NO: 44;
(f) a heavy chain variable region comprising the amino acid sequence set forth in SEQ ID NO: 52, and a light chain variable region comprising the amino acid sequence set forth in SEQ ID NO: 53;
(g) a heavy chain variable region comprising the amino acid sequence set forth in SEQ ID NO: 62, and a light chain variable region comprising the amino acid sequence set forth in SEQ ID NO: 63;
(h) a heavy chain variable region comprising the amino acid sequence set forth in SEQ ID NO: 72, and a light chain variable region comprising the amino acid sequence set forth in SEQ ID NO: 73;
(i) a heavy chain variable region comprising the amino acid sequence set forth in SEQ ID NO: 81, and a light chain variable region comprising the amino acid sequence set forth in SEQ ID NO: 82;
(j) a heavy chain variable region comprising the amino acid sequence set forth in SEQ ID NO: 88, and a light chain variable region comprising the amino acid sequence set forth in SEQ ID NO: 89; and
(k) a heavy chain variable region comprising the amino acid sequence set forth in SEQ ID NO: 8, and a light chain variable region comprising the amino acid sequence set forth in SEQ ID NO: 92.

In certain embodiments, the presently disclosed subject matter provides antibodies or antigen-binding fragments thereof that comprise the heavy chain and light chain CDR1s, CDR2s and CDR3s of 3-P14, 4-B2, 1-J19, 1-J19-2, 1-P13, 1-P23, 1-A20, 2-N3, 1-H19, 2-F18, and 4-P3.

The amino acid sequences of the V_{H} CDR1s of 3-P14, 4-B2, 1-J19, 1-J19-2, 1-P13, 1-P23, 1-A20, 2-N3, 1-H19, 2-F18, and 4-P3 are set forth in SEQ ID NOs: 2, 12, 22, 32, 37, 47, 56, 66, 76, and 85. The amino acid sequences of the V_{H} CDR2s of 3-P14, 4-B2, 1-J19, 1-J19-2, 1-P13, 1-P23, 1-A20, 2-N3, 1-H19, 2-F18, and 4-P3 antibodies are set forth in SEQ ID NOs: 3, 13, 23, 33, 38, 57, 67, and 86. The amino acid sequences of the V_{H} CDR3s of 3-P14, 4-B2, 1-J19, 1-J19-2, 1-P13, 1-P23, 1-A20, 2-N3, 1-H19, 2-F18, and 4-P3 are set forth in SEQ ID NOs: 4, 14, 24, 34, 39, 48, 58, 68, 77, and 87.

The amino acid sequences of the V_{L} CDR1s of 3-P14, 4-B2, 1-J19, 1-J19-2, 1-P13, 1-P23, 1-A20, 2-N3, 1-H19, 2-F18, and 4-P3 are set forth in SEQ ID NOs: 5, 15, 25, 40, 49, 59, 69, and 78. The amino acid sequences of the V_{L} CDR2s of 3-P14, 4-B2, 1-J19, 1-J19-2, 1-P13, 1-P23, 1-A20, 2-N3, 1-H19, 2-F18, and 4-P3 are set forth in SEQ ID NOs: 6, 16, 26, 41, 50, 60, 70, and 79. The amino acid sequences of the V_{L} CDR3s of 3-P14, 4-B2, 1-J19, 1-J19-2, 1-P13, 1-P23, 1-A20, 2-N3, 1-H19, 2-F18, and 4-P3 are set forth in SEQ ID NOs: 7, 17, 27, 42, 51, 61, 71, and 80. The CDR regions are delineated using the IMGT system. In certain embodiments, the CDR regions are delineated using the IMGT numbering system accessible at http://www.imgt.org/IMGT_vquest/input.

Given that each of these antibodies or antigen-binding fragments thereof can bind to CD33 and that antigen-binding specificity is provided primarily by the CDR1, CDR2, and CDR3 regions, the V_{H} CDR1, CDR2, and CDR3 sequences and V_{L} CDR1, CDR2, and CDR3 sequences can be “mixed and matched” (i.e., CDRs from different antibodies can be mixed and match, although each antibody must contain a V_{H} CDR1, CDR2, and CDR3 and a V_{L} CDR1, CDR2, and CDR3) to create other anti-CD33 binding molecules. CD33 binding of such “mixed and matched” antibodies can be tested using the binding assays described above. When V_{H} CDR sequences are mixed and matched, the CDR1, CDR2 and/or CDR3 sequence from a particular V_{H} sequence is replaced with a structurally similar CDR sequence(s). Likewise, when V_{L} CDR sequences are mixed and matched, the CDR1, CDR2 and/or CDR3 sequence from a particular V_{L} sequence preferably is replaced with a structurally similar CDR sequence(s). It will be readily apparent to the ordinarily skilled artisan that novel V_{H} and V_{L} sequences can be created by substituting one or more V_{H} and/or V_{L} CDR region sequences with structurally similar sequences from the CDR sequences of the antibodies or antigen-binding fragments thereof disclosed herein 3-P14, 4-B2, 1-J19, 1-J19-2, 1-P13, 1-P23, 1-A20, 2-N3, 1-H19, 2-F18, and 4-P3.

In certain embodiments, the presently disclosed subject matter provides an antibody or an antigen-binding fragment thereof comprising:
(a) a heavy chain variable region CDR1 comprising the amino acid sequence set forth in SEQ ID NO: 2, SEQ ID NO: 12, SEQ ID NO: 22, SEQ ID NO: 32, SEQ ID NO: 37, SEQ ID NO: 47, SEQ ID NO: 56, SEQ ID NO: 66, SEQ ID NO: 76, or SEQ ID NO: 85;
(b) a heavy chain variable region CDR2 comprising the amino acid sequence set forth in SEQ ID NO: 3, SEQ ID NO: 13, SEQ ID NO: 23, SEQ ID NO: 33, SEQ ID NO: 38, SEQ ID NO: 57, SEQ ID NO: 67, or SEQ ID NO: 86;
(c) a heavy chain variable region CDR3 comprising the amino acid sequence set forth in SEQ ID NO: 4, SEQ ID NO: 14, SEQ ID NO: 24, SEQ ID NO: 34, SEQ ID NO: 39, SEQ ID NO: 48, SEQ ID NO: 58, SEQ ID NO: 68, SEQ ID NO: 77, or SEQ ID NO: 87;
(d) a light chain variable region CDR1 comprising the amino acid sequence set forth in SEQ ID NO: 5, SEQ ID NO: 15, SEQ ID NO: 25, SEQ ID NO: 40, SEQ ID NO: 49, SEQ ID NO: 59, SEQ ID NO: 69, or SEQ ID NO: 78;
(e) a light chain variable region CDR2 comprising the amino acid sequence set forth in SEQ ID NO: 6, SEQ ID NO: 16, SEQ ID NO: 26, SEQ ID NO: 41, SEQ ID NO: 50, SEQ ID NO: 60, SEQ ID NO: 70, or SEQ ID NO: 79; and
(f) a light chain variable region CDR3 comprising the amino acid sequence set forth in SEQ ID NO: 7, SEQ ID NO: 17, SEQ ID NO: 27, SEQ ID NO: 42, SEQ ID NO: 51, SEQ ID NO: 61, SEQ ID NO: 71, or SEQ ID NO: 80.

In certain embodiments, the antibody or antigen-binding fragment thereof comprises:
(a) a heavy chain variable region CDR1 comprising the amino acid sequence set forth in SEQ ID NO: 2;
(b) a heavy chain variable region CDR2 comprising the amino acid sequence set forth in SEQ ID NO: 3;
(c) a heavy chain variable region CDR3 comprising the amino acid sequence set forth in SEQ ID NO: 4;
(d) a light chain variable region CDR1 comprising the amino acid sequence set forth in SEQ ID NO: 5;
(e) a light chain variable region CDR2 comprising the amino acid sequence set forth in SEQ ID NO: 6; and
(f) a light chain variable region CDR3 comprising the amino acid sequence set forth in SEQ ID NO: 7.

In certain embodiments, the antibody or antigen-binding fragment thereof comprises:
(a) a heavy chain variable region CDR1 comprising the amino acid sequence set forth in SEQ ID NO: 12;
(b) a heavy chain variable region CDR2 comprising the amino acid sequence set forth in SEQ ID NO: 13;
(c) a heavy chain variable region CDR3 comprising the amino acid sequence set forth in SEQ ID NO: 14;
(d) a light chain variable region CDR1 comprising the amino acid sequence set forth in SEQ ID NO: 15;
(e) a light chain variable region CDR2 comprising the amino acid sequence set forth in SEQ ID NO: 16; and
(f) a light chain variable region CDR3 comprising the amino acid sequence set forth in SEQ ID NO: 17.

In certain embodiments, the antibody or antigen-binding fragment thereof comprises:
(a) a heavy chain variable region CDR1 comprising the amino acid sequence set forth in SEQ ID NO: 22;
(b) a heavy chain variable region CDR2 comprising the amino acid sequence set forth in SEQ ID NO: 23;
(c) a heavy chain variable region CDR3 comprising the amino acid sequence set forth in SEQ ID NO: 24;
(d) a light chain variable region CDR1 comprising the amino acid sequence set forth in SEQ ID NO: 25;
(e) a light chain variable region CDR2 comprising the amino acid sequence set forth in SEQ ID NO: 26; and
(f) a light chain variable region CDR3 comprising the amino acid sequence set forth in SEQ ID NO: 27.

In certain embodiments, the antibody or antigen-binding fragment thereof comprises:
(a) a heavy chain variable region CDR1 comprising the amino acid sequence set forth in SEQ ID NO: 32;
(b) a heavy chain variable region CDR2 comprising the amino acid sequence set forth in SEQ ID NO: 33;
(c) a heavy chain variable region CDR3 comprising the amino acid sequence set forth in SEQ ID NO: 34;
(d) a light chain variable region CDR1 comprising the amino acid sequence set forth in SEQ ID NO: 25;
(e) a light chain variable region CDR2 comprising the amino acid sequence set forth in SEQ ID NO: 26; and
(f) a light chain variable region CDR3 comprising the amino acid sequence set forth in SEQ ID NO: 27.

In certain embodiments, the antibody or antigen-binding fragment thereof comprises:
(a) a heavy chain variable region CDR1 comprising the amino acid sequence set forth in SEQ ID NO: 37;
(b) a heavy chain variable region CDR2 comprising the amino acid sequence set forth in SEQ ID NO: 38;
(c) a heavy chain variable region CDR3 comprising the amino acid sequence set forth in SEQ ID NO: 39;
(d) a light chain variable region CDR1 comprising the amino acid sequence set forth in SEQ ID NO: 40;
(e) a light chain variable region CDR2 comprising the amino acid sequence set forth in SEQ ID NO: 41; and
(f) a light chain variable region CDR3 comprising the amino acid sequence set forth in SEQ ID NO: 42.

In certain embodiments, the antibody or antigen-binding fragment thereof comprises:
(a) a heavy chain variable region CDR1 comprising the amino acid sequence set forth in SEQ ID NO: 47;
(b) a heavy chain variable region CDR2 comprising the amino acid sequence set forth in SEQ ID NO: 38;
(c) a heavy chain variable region CDR3 comprising the amino acid sequence set forth in SEQ ID NO: 48;
(d) a light chain variable region CDR1 comprising the amino acid sequence set forth in SEQ ID NO: 49;
(e) a light chain variable region CDR2 comprising the amino acid sequence set forth in SEQ ID NO: 50; and
(f) a light chain variable region CDR3 comprising the amino acid sequence set forth in SEQ ID NO: 51.

In certain embodiments, the antibody or antigen-binding fragment thereof comprises:
(a) a heavy chain variable region CDR1 comprising the amino acid sequence set forth in SEQ ID NO: 56;
(b) a heavy chain variable region CDR2 comprising the amino acid sequence set forth in SEQ ID NO: 57;
(c) a heavy chain variable region CDR3 comprising the amino acid sequence set forth in SEQ ID NO: 58;
(d) a light chain variable region CDR1 comprising the amino acid sequence set forth in SEQ ID NO: 59;
(e) a light chain variable region CDR2 comprising the amino acid sequence set forth in SEQ ID NO: 60; and
(f) a light chain variable region CDR3 comprising the amino acid sequence set forth in SEQ ID NO: 61.

In certain embodiments, the antibody or antigen-binding fragment thereof comprises:
(a) a heavy chain variable region CDR1 comprising the amino acid sequence set forth in SEQ ID NO: 66;
(b) a heavy chain variable region CDR2 comprising the amino acid sequence set forth in SEQ ID NO: 67;
(c) a heavy chain variable region CDR3 comprising the amino acid sequence set forth in SEQ ID NO: 68;
(d) a light chain variable region CDR1 comprising the amino acid sequence set forth in SEQ ID NO: 69;
(e) a light chain variable region CDR2 comprising the amino acid sequence set forth in SEQ ID NO: 70; and
(f) a light chain variable region CDR3 comprising the amino acid sequence set forth in SEQ ID NO: 71.

In certain embodiments, the antibody or antigen-binding fragment thereof comprises:
(a) a heavy chain variable region CDR1 comprising the amino acid sequence set forth in SEQ ID NO: 76;
(b) a heavy chain variable region CDR2 comprising the amino acid sequence set forth in SEQ ID NO: 57;
(c) a heavy chain variable region CDR3 comprising the amino acid sequence set forth in SEQ ID NO: 77;
(d) a light chain variable region CDR1 comprising the amino acid sequence set forth in SEQ ID NO: 78;
(e) a light chain variable region CDR2 comprising the amino acid sequence set forth in SEQ ID NO: 79; and
(f) a light chain variable region CDR3 comprising the amino acid sequence set forth in SEQ ID NO: 80.

In certain embodiments, the antibody or antigen-binding fragment thereof comprises:
(a) a heavy chain variable region CDR1 comprising the amino acid sequence set forth in SEQ ID NO: 85;
(b) a heavy chain variable region CDR2 comprising the amino acid sequence set forth in SEQ ID NO: 86;
(c) a heavy chain variable region CDR3 comprising the amino acid sequence set forth in SEQ ID NO: 87;
(d) a light chain variable region CDR1 comprising the amino acid sequence set forth in SEQ ID NO: 5;
(e) a light chain variable region CDR2 comprising the amino acid sequence set forth in SEQ ID NO: 41; and
(f) a light chain variable region CDR3 comprising the amino acid sequence set forth in SEQ ID NO: 42.

In certain embodiments, the anti-CD33 antibody or antigen-binding fragment thereof comprises a heavy chain constant region and/or a light chain constant region.

In certain embodiments, the heavy chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 95. In certain embodiments, the heavy chain constant region comprises the amino acid sequence set forth in SEQ ID NO: 95. SEQ ID NO: 95 is provided below.

[SEQ ID NO: 95]
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

In certain embodiments, the light chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 96. In certain embodiments, the light chain constant region comprises the amino acid sequence set forth in SEQ ID NO: 96. SEQ ID NO: 96 is provided below.

[SEQ ID NO: 96]
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC

In certain embodiments, the light chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 99. In certain embodiments, the light chain constant region comprises the amino acid sequence set forth in SEQ ID NO: 99. SEQ ID NO: 99 is provided below.

[SEQ ID NO: 99]
GQPKANPTVTLFPPSSEELQANKATLVCLISDFYPGAVTVAWKADGSPV
KAGVETTKPSKQSNNKYAASSYLSLTPEQWKSHRSYSCQVTHEGSTVEK
TVAPTECS

In certain embodiments, the anti-CD33 antibody or an antigen-binding fragment thereof comprises: (a) a heavy chain constant region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 95; and (b) a light chain constant region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 96. In certain embodiments, the anti-CD33 antibody or an antigen-binding fragment thereof comprises: (a) a heavy chain constant region comprising the amino acid sequence set forth in SEQ ID NO: 95; and (b) a light chain constant region comprising the amino acid sequence set forth in SEQ ID NO: 96.

In certain embodiments, the anti-CD33 antibody or an antigen-binding fragment thereof comprises: (a) a heavy chain constant region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 95; and (b) a light chain constant region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 99. In certain embodiments, the anti-CD33 antibody or an antigen-binding fragment thereof comprises: (a) a heavy chain constant region comprising the amino acid sequence set forth in SEQ ID NO: 95; and (b) a light chain constant region comprising the amino acid sequence set forth in SEQ ID NO: 99.

In certain embodiments, the anti-CD33 antibody or an antigen-binding fragment thereof comprises a heavy chain comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 97. In certain embodiments, the anti-CD33 antibody or an antigen-binding fragment thereof comprises a heavy chain comprising the amino acid sequence set forth in SEQ ID NO: 97. SEQ ID NO: 97 is provided below.

[SEQ ID NO: 97]
EVQLLESGGGLVQPGGSLRLSCAASGFTFSTYAMSWVRQAPGKGLEWVS
AISGRGGSTYYTDSVKGRFTISRDNSKNTVSLQMNSLRAEDTAVYYCAG
RGDYYYYYGMDVWGQGTTVTVSAASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYT
QKSLSLSPGK

In certain embodiments, the anti-CD33 antibody or an antigen-binding fragment thereof comprises a light chain comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 98. In certain embodiments, the anti-CD33 antibody or an antigen-binding fragment thereof comprises a light chain comprising the amino acid sequence set forth in SEQ ID NO: 98. SEQ ID NO: 98 is provided below.

[SEQ ID NO: 98]
DIVMTQSPLSSPVTLGQPASFSCRSSQSLVYSDGNTYLSWLQQRPGQPP
RLLIYKISNRFSGVPDRFSGSGAGTDFTLKISRVEAEDVGVYYCMQSTQ
FPHTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSENRGEC

In certain embodiments, the anti-CD33 antibody or an antigen-binding fragment thereof comprises: (a) a heavy chain comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 97; and (b) a light chain comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 98. In certain embodiments, the anti-CD33 antibody or an antigen-binding fragment thereof comprises: (a) a heavy chain comprising the amino acid sequence set forth in SEQ ID NO: 97; and (b) a light chain comprising the amino acid sequence set forth in SEQ ID NO: 98.

In certain embodiments, the anti-CD33 antibody or an antigen-binding fragment thereof comprises a heavy chain comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 100. In certain embodiments, the anti-CD33 antibody or an antigen-binding fragment thereof comprises a heavy chain comprising the amino acid sequence set forth in SEQ ID NO: 100. SEQ ID NO: 100 is provided below.

[SEQ ID NO: 100]
EVHLLESGGGLVQPGGSLRLSCAASGFIFSSNAMSWVRQAPGKGLEWVS
AISGYGGNTYYADSVKGRFTISRDNSKNTLYLQMNSLRAEDTAVYYCAK
WGTYIVGATGDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYT
QKSLSLSPGK

In certain embodiments, the anti-CD33 antibody or an antigen-binding fragment thereof comprises a light chain comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 101. In certain embodiments, the anti-CD33 antibody or an antigen-binding fragment thereof comprises a light chain comprising the amino acid sequence set forth in SEQ ID NO: 101. SEQ ID NO: 101 is provided below.

[SEQ ID NO: 101]
QSALTQPPSASGSPGQSVTISCTGTSNDVGGYNYVSWYQQHPGKAPKLL
IYEVSKRPSGVPDRFSGSQSGNTASLTVSGLQAEDEADYYCSSYAGSNN
WVFGGGTKLTVLGQPKANPTVTLFPPSSEELQANKATLVCLISDFYPGA
VTVAWKADGSPVKAGVETTKPSKQSNNKYAASSYLSLTPEQWKSHRSYS
CQVTHEGSTVEKTVAPTECS

In certain embodiments, the anti-CD33 antibody or an antigen-binding fragment thereof comprises: (a) a heavy chain comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 100; and (b) a light chain comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 101. In certain embodiments, the anti-CD33 antibody or an antigen-binding fragment thereof comprises: (a) a heavy chain comprising the amino acid sequence set forth in SEQ ID NO: 100; and (b) a light chain comprising the amino acid sequence set forth in SEQ ID NO: 101.

In certain embodiments, the anti-CD33 antibody or an antigen-binding fragment thereof comprises a heavy chain comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 102. In certain embodiments, the anti-CD33 antibody or an antigen-binding fragment thereof comprises a heavy chain comprising the amino acid sequence set forth in SEQ ID NO: 102. SEQ ID NO: 102 is provided below.

[SEQ ID NO: 102]
QVQLQQSGPGLVKPSQTLSLTCAISGDSVSSNSAAWNWIRQSPSRGLEW
LGRTYFRSKWYNVYAVSVKSRITINPDTSKNQFSLQLNSVTPEDTAVYY
CASEGGSYYDHWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK

In certain embodiments, the anti-CD33 antibody or an antigen-binding fragment thereof comprises a light chain comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 103. In certain embodiments, the anti-CD33 antibody or an antigen-binding fragment thereof comprises a light chain comprising the amino acid sequence set forth in SEQ ID NO: 103. SEQ ID NO: 103 is provided below.

[SEQ ID NO: 103]
DIQMTQSPSSVSASVGDRVTITCRASQGISNWLTWYQQKPGKAPKLLIY
AASSLQSGVPSRFSGSGSGTDFTLTISSLQPEDFATYYCQQADSFPFTF
GPGTKVDIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

In certain embodiments, the anti-CD33 antibody or an antigen-binding fragment thereof comprises: (a) a heavy chain comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 100; and (b) a light chain comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 102. In certain embodiments, the anti-CD33 antibody or an antigen-binding fragment thereof comprises: (a) a heavy chain comprising the amino acid sequence set forth in SEQ ID NO: 100; and (b) a light chain comprising the amino acid sequence set forth in SEQ ID NO: 103.

The constant regions/framework regions of the anti-CD33 antibodies disclosed herein can be altered, for example, by amino acid substitution, to modify the properties of the antibody (e.g., to increase or decrease one or more of: antigen binding affinity, Fc receptor binding, antibody carbohydrate, for example, glycosylation, fucosylation etc., the number of cysteine residues, effector cell function, effector cell function, complement function or introduction of a conjugation site).

In certain embodiments, a presently disclosed anti-CD33 antibody is a fully-human antibody, e.g., any one of 3-P14, 4-B2, 1-J19, 1-J19-2, 1-P13, 1-P23, 1-A20, 2-N3, 1-H19, 2-F18, and 4-P3. Fully-human mAbs, when administered to humans, causing serious side effects, including anaphylaxis and hypersensitivity reactions.

The use of phage display libraries has made it possible to select large numbers of antibody repertoires for unique and rare Abs against very defined epitopes (for more details on phage display see McCafferty et al., Phage antibodies: filamentous phage displaying antibody variable domains. Nature, 348: 552-554.) The rapid identification of human Fab or single chain Fv (scFv) fragments highly specific for tumor antigen-derived peptide-MHC complex molecules has thus become possible. In addition, by engineering full-length monoclonal antibody (mAb) using the Fab fragments, it is possible to directly generate a therapeutic human mAb, bypassing months of time-consuming work, normally needed for developing therapeutic mAbs. The presently disclosed subject matter involves the development of a fully human mAb that recognizes, for example, a human CD33 polypeptide (e.g., a polypeptide having the amino acid sequence set forth in SEQ ID NO: 116) for cancer therapy.

### 5.3.3. Homologous Antibodies

In certain embodiments, a presently disclosed anti-CD33 antibody or antigen-binding fragment thereof comprises heavy and light chain variable regions comprising amino acid sequences that are homologous or identical to the amino acid sequences of the antibodies described herein (e.g., 3-P14, 4-B2, 1-J19, 1-J19-2, 1-P13, 1-P23, 1-A20, 2-N3, 1-H19, 2-F18, and 4-P3 antibodies), and wherein the antibodies or antigen-binding fragments thereof retain the desired functional properties of the anti-CD33 antibodies or antigen-binding fragments thereof of the presently disclosed subject matter.

For example, the presently disclosed subject matter provides an anti-CD33 antibody or an antigen-binding fragment thereof, comprising a heavy chain variable region and a light chain variable region, wherein:
(a) the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 8, SEQ ID NO: 18, SEQ ID NO: 28, SEQ ID NO: 35, SEQ ID NO: 43, SEQ ID NO: 52, SEQ ID NO: 62, SEQ ID NO: 72, SEQ ID NO: 81, or SEQ ID NO: 88; and
(b) the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 9, SEQ ID NO: 19, SEQ ID NO: 29, SEQ ID NO: 44, SEQ ID NO: 53, SEQ ID NO: 63, SEQ ID NO: 73, SEQ ID NO: 82, SEQ ID NO: 89, or SEQ ID NO: 92.

In certain embodiments, the V_{H} and/or V_{L} amino acid sequences can be at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the sequences set forth above. An antibody having V_{H} and V_{L} regions having high (i.e., 80% or greater) homology or identity to the V_{H} and V_{L} regions of the sequences set forth above, can be obtained by mutagenesis (e.g., site-directed or PCR-mediated mutagenesis), followed by testing of the encoded altered antibody for retained function (i.e., the binding affinity) using the binding assays described herein.

In certain embodiments, a presently disclosed anti-CD33 antibody or antigen-binding fragment thereof comprises heavy and light chain constant regions comprising amino acid sequences that are homologous or identical to the amino acid sequences of the antibodies described herein (e.g., 3-P14, 4-B2, 1-J19, 1-J19-2, 1-P13, 1-P23, 1-A20, 2-N3, 1-H19, 2-F18, and 4-P3 antibodies), and wherein the antibodies or antigen-binding fragments thereof retain the desired functional properties of the anti-CD33 antibodies or antigen-binding fragments thereof of the presently disclosed subject matter.

For example, the presently disclosed subject matter provides an anti-CD33 antibody or an antigen-binding fragment thereof, comprising a heavy chain constant region and a light chain constant region, wherein:
(a) the heavy chain constant region (C_{H}) comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 95; and
(b) the light chain constant region (C_{L}) comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 9, SEQ ID NO: 96 or SEQ ID NO: 99.

In certain embodiments, the C_{H} and/or C_{L} amino acid sequences can be at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the sequences set forth above. An antibody having C_{H} and C_{L} regions having high (i.e., 80% or greater) homology or identity to the C_{H} and C_{L} regions of the sequences set forth above, can be obtained by mutagenesis (e.g., site-directed or PCR-mediated mutagenesis), followed by testing of the encoded altered antibody for retained function (i.e., the binding affinity) using the binding assays described herein.

As used herein, the percent homology between two amino acid sequences is equivalent to the percent identity between the two sequences. The percent identity or homology between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent homology or identity between two amino acid sequences can be determined using the algorithm of E. Meyers and W. Miller (Comput Appl Biosci (1988); 14:11-17) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J Mol Biol (1970); 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the presently disclosed subject matter can further be used as a “query sequence” to perform a search against public databases to, for example, identify related sequences. Such searches can be performed using the XBLAST program (version 2.0) of Altschul et al., J Mol Biol (1990); 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., Nucleic Acids Res (1997); 25(17):3389-3402. When utilizing BLAST and Gapped BLAST programs, the default parameters of the respective programs (e.g., XBLAST and NBLAST) can be used.

5.3.4. Antibodies with Conservative Modifications

In certain embodiments, a presently disclosed anti-CD33 antibody or an antigen-binding fragment thereof comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on the preferred antibodies described herein (e.g., 3-P14, 4-B2, 1-J19, 1-J19-2, 1-P13, 1-P23, 1-A20, 2-N3, 1-H19, 2-F18, and 4-P3 antibodies), or a conservative modification thereof, and wherein the antibodies retain the desired functional properties of the anti-CD33 antibodies or antigen-binding fragments thereof of the presently disclosed subject matter. The presently disclosed subject matter provides an antibody or an antigen-binding fragment thereof, comprising a heavy chain variable region comprising CDR1, CDR2, and CDR3 sequences and a light chain variable region comprising CDR1, CDR2, and CDR3 sequences, wherein:
(a) the heavy chain variable region CDR3 sequence comprises an amino acid sequence selected from SEQ ID NOs: 4, 14, 24, 34, 39, 48, 58, 68, 77, and 87, and conservative modifications thereof;
(b) the light chain variable region CDR3 sequence comprises an amino acid sequence selected from SEQ ID NOs: 7, 17, 27, 42, 51, 61, 71, and 80, and conservative modifications thereof.

In certain embodiments, the heavy chain variable region CDR3 sequence comprises an amino acid sequence selected from SEQ ID NOs: 4, 14, 24, 34, 39, 48, 58, 68, 77, and 87, and conservative modifications thereof; and the light chain variable region CDR3 sequence comprises an amino acid sequence selected from SEQ ID NOs: 7, 17, 27, 42, 51, 61, 71, and 80, and conservative modifications thereof.

In certain embodiments, the heavy chain variable region CDR2 sequence comprises an amino acid sequence selected from SEQ ID NOs: 3, 13, 23, 33, 38, 57, 67, and 86, and conservative modifications thereof; and the light chain variable region CDR2 sequence comprises an amino acid sequence selected from SEQ ID NOs: 6, 16, 26, 41, 50, 60, 70, and 79, and conservative modifications thereof.

In certain embodiments, the heavy chain variable region CDR1 sequence comprises an amino acid sequence selected from SEQ ID NOs: 2, 12, 22, 32, 37, 47, 56, 66, 76, and 85, and conservative modifications thereof; and the light chain variable region CDR1 sequence comprises an amino acid sequence selected from SEQ ID NOs: 5, 15, 25, 40, 49, 59, 69, and 78, and conservative modifications thereof.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the present disclosure by standard techniques known in the art, such as site-directed mutagenesis and PCR-mediated mutagenesis.

Conservative amino acid substitutions are ones in which the amino acid residue is replaced with an amino acid residue having a similar side chain. Families of amino acid residues having similar side chains have been defined in the art. Exemplary conservative amino acid substitutions are shown in Table 24. Amino acid substitutions may be introduced into an antibody of interest and the products screened for a desired activity, e.g., retained/improved antigen binding, decreased immunogenicity, or improved ADCC or CDC. In certain embodiments, a sequence disclosed herein, e.g., a CDR sequence, a V_{H} sequence or a V_{L} sequence, can have up to about one, up to about two, up to about three, up to about four, up to about five, up to about six, up to about seven, up to about eight, up to about nine or up to about ten amino acid residues that are modified and/or substituted.

TABLE 12

| Original Residue | Exemplary conservative amino acid Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala |

Amino acids may be grouped according to common side-chain properties:
hydrophobic: Norleucine, Met, Ala, Val, Leu, Ile;
neutral hydrophilic: Cys, Ser, Thr, Asn, Gln;
acidic: Asp, Glu;
basic: His, Lys, Arg;
residues that influence chain orientation: Gly, Pro;
aromatic: Trp, Tyr, Phe.

Non-conservative substitutions will entail exchanging a member of one of these classes for another class.

5.3.5. Anti-CD33 Antibodies that Cross-Compete for Binding to CD33 with Anti-CD33Antibodies of the Invention

The presently disclosed subject matter provides antibodies or antigen-binding fragments thereof that cross-compete with any of the disclosed anti-CD33 antibodies for binding to CD33 (e.g., human CD33). For example, and not by way of limitation, the cross-competing antibodies can bind to the same epitope region, e.g., same epitope, adjacent epitope, or overlapping as any of the anti-CD33 antibodies or antigen-binding fragments thereof of the presently disclosed subject matter. In certain embodiments, the reference antibody or reference antigen-binding fragments thereof for cross-competition studies can be any one of the anti-CD33 antibodies or antigen-binding fragments thereof disclosed herein, e.g., 3-P14, 4-B2, 1-J19, 1-J19-2, 1-P13, 1-P23, 1-A20, 2-N3, 1-H19, 2-F18, and 4-P3 antibodies.

Such cross-competing antibodies can be identified based on their ability to cross-compete with any one of the presently disclosed anti-CD33 antibodies or antigen-binding fragments thereof in standard CD33 binding assays. For example, Biacore analysis, ELISA assays or flow cytometry can be used to demonstrate cross-competition with the antibodies of the presently disclosed subject matter. The ability of a test antibody to inhibit the binding of, for example, any one of the presently disclosed anti-CD33 antibodies (e.g., 3-P14, 4-B2, 1-J19, 1-J19-2, 1-P13, 1-P23, 1-A20, 2-N3, 1-H19, 2-F18, and 4-P3 antibodies) to CD33 (e.g., human CD33) demonstrates that the test antibody can compete with any one of the presently disclosed anti-CD33 antibodies or antigen-binding fragments thereof for binding to CD33 (e.g., human CD33) and thus binds to the same epitope region on CD33 (e.g., human CD33) as any one of the presently disclosed anti-CD33 antibodies or antigen-binding fragments thereof. In certain embodiments, the cross-competing antibody or antigen-binding fragment thereof binds to the same epitope on CD33 (e.g., human CD33) as any one of the presently disclosed anti-CD33 antibodies or antigen-binding fragments thereof.

### 5.3.6. Characterization of Antibody Binding to Antigen

Antibodies or antigen-binding fragments thereof of the presently disclosed subject can be tested for binding to CD33 by, for example, standard ELISA. To determine if the selected anti-CD33 antibodies bind to unique epitopes, each antibody can be biotinylated using commercially available reagents (Pierce, Rockford, IL). Competition studies using unlabeled monoclonal antibodies and biotinylated monoclonal antibodies can be performed using CD33 coated-ELISA plates as described above. Biotinylated mAb binding can be detected with a strep-avidin-alkaline phosphatase probe.

To determine the isotype of purified antibodies, isotype ELISAs can be performed using reagents specific for antibodies of a particular isotype. Anti-CD33 human IgGs can be further tested for reactivity with CD33 antigen by Western blotting.

In certain embodiments, the K_{D} is measured by a radiolabeled antigen binding assay (RIA). In certain embodiments, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J Mol Biol (1999); 293:865-881).

In certain embodiments, the K_{D} is measured using a BIACORE® surface plasmon resonance assay. For example, an assay using a BIACORE®-2000 or a BIACORE®-3000 (BIAcore, Inc., Piscataway, NJ)

### 5.3.7. Immunoconjugates

The presently disclosed subject provides an anti-CD33 antibody or an antigen-binding fragment thereof, conjugated to a therapeutic moiety, such as a cytotoxin, a drug (e.g., an immunosuppressant) or a radiotoxin. Such conjugates are referred to herein as “immunoconjugates”. Immunoconjugates that include one or more cytotoxins are referred to as “immunotoxins.” A cytotoxin or cytotoxic agent includes any agent that is detrimental to (e.g., kills) cells. Non-limiting Examples of cytotoxins include taxol (such as ricin, diphtheria, gelonin), cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, calecheamicin, aureastatin, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), hypomethylating agents (azacytidine and decitabine), and anti-mitotic agents (e.g., vincristine and vinblastine).

Other examples of therapeutic cytotoxins that can be conjugated to an anti-CD33 antibody disclosed herein include duocarmycins, calicheamicins, maytansines and auristatins, and derivatives thereof. Cytotoxins can be conjugated to an anti-CD33 antibody or an antigen-binding fragment thereof disclosed herein using linker technology available in the art. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D). For further discussion of types of cytotoxins, linkers and methods for conjugating therapeutic agents to antibodies, see also Saito, G. et al. (2003) Adv. Drug Deliv. Rev. 55:199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3:1089-1091; Senter, P. D. and Springer, C. J. (2001) Adv. Drug Deliv. Rev. 53:247-264.

Anti-CD33 antibodies or antigen-binding fragments thereof of the presently disclosed subject matter also can be conjugated to a radioactive isotope to generate cytotoxic radiopharmaceuticals, also referred to as radioimmunoconjugates. Non-limiting examples of radioactive isotopes that can be conjugated to antibodies for use diagnostically or therapeutically include ⁴⁷Sc, ⁶⁷Cu, ⁹⁰Y, ¹³¹I, ¹⁴⁹Th, ¹⁶¹Tb, ¹⁷⁷Lu, ²²⁵ Ac, ²¹³Bi, ²²³Ra and ²²⁷Th. Methods for preparing radioimmunconjugates are established in the art. Examples of radioimmunoconjugates are commercially available, including Zevalin™ (IDEC Pharmaceuticals) and Bexxar™ (Corixa Pharmaceuticals), and similar methods can be used to prepare radioimmunoconjugates using the presently disclosed anti-CD33 antibodies.

In certain embodiments, the anti-CD33 antibodies or antigen-binding fragments thereof of the presently disclosed subject matter can be conjugated to a radioisotope to generate a radioimmunoconjugate by using a chelator. As used herein, the term “chelator” refers to a chemical compound in the form of a heterocyclic ring or surrounding structure containing a metal ion attached by coordinate bonds to at least two nonmetal ions. Non-limiting examples of chelator include 1,4,7-Triazacyclononane-1,4,7-triacetic acid (NOTA), 2,2′-(7-(1-carboxy-4-((4-isothiocyanatobenzyl)amino)-4-oxobutyl)-1,4,7-triazonane-1,4-diyl)diacetic acid (NODA), 2,2′,2″,2″-(1,4,7,10-Tetraazacyclododecane-1,4,7,10-tetrayl)tetraacetic acid (DOTA), Diethylenetriamine-N,N,N′,N,N-pentaacetic acid, pentetic acid, (Carboxymethyl)imino]bis(ethylenenitrilo)-tetra-acetic acid (DTPA), 1-Hydroxy-2-pyridone; 2-Pyridinol-1-oxide (HOPO), N-(5-(3-((5-Aminopentyl)hydroxycarbamoyl) propionamido)pentyl)-3-((5-(N-hydroxyacetamido) pentyl)carbamoyl) propionohydroxamic acid (DFO), and 2-[1,4,7-Triazacyclononan-1-yl-4,7-bis(tBu-ester)]-1,5-pentanedioic acid (NODAGA). Additional exemplary chelators encompassed by the presently disclosed subject matter include AAZTA and derivatives thereof, BAT, BARAC, BPCA, TE2A, CB-TE2A, CB0TE1A1P, CB-TE2P, MM-TE2A, DM TE-2A, CP356, DATA, DBCO, DiAmSar and derivatives thereof, DIBO, DIMA, DFO, DGO, DOTA and derivatives thereof (e.g., Ac-DOTA, benzo-DOTA, dibenzo-DOTA, CB-DO2A, 3p-C-DEPA, Oxo-DO3A), DOTMA derivative thereof (e.g., benzo-DOTMA), DTPA and derivatives thereof (e.g., benzo-DTPA, dibenzo-DTPA, phenyl-DTPA, diphenyl-DTPA, benzyl-DTPA, dibenzyl-DTPA, 1B4M-DTPA, CHX-A″-DTPA), EDTA, EGTA, EHPG and derivatives thereof (e.g., 5-C1-EHPG, 5-Br-EHPG, 5-Me-EHPG, 5t-Bu-EHPG, 5-sec-Bu-EHPG), H2dedpa, H4octapa, H2azapa, H5decapa, H6phospa, HBED and derivatives thereof, SHBED, HEHA, HYNIC, LICAM and derivatives thereof, MECAM, NODASA, NODAGA, NOPO, NOTA and derivatives thereof (e.g., benzo-NOTA), NETA, PEPA, PCTA, PDTA, TACN-TM, TCMC, TETA and derivatives thereof (e.g., benzo-TETA), TETMA and derivatives (e.g., benzo-TETMA), TRAP (PRP9), TRITA, TTHA and derivatives thereof. The antibody conjugates of the presently disclosed subject matter can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor (TNF) or interferon-γ; or, biological response modifiers such as, for example, lymphokines, interleukin-1 (IL-1), interleukin-2 (IL-2), interleukin-6 (IL-6), granulocyte macrophage colony stimulating factor (GM-CSF), granulocyte colony stimulating factor (G-CSF), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

### 5.3.8. Multi-Specific Molecules

The presently disclosed subject matter provides multi-specific molecules comprising an anti-CD33 antibody, or a fragment thereof, disclosed herein. A presently disclosed or an antigen-binding fragment thereof can be derivatized or linked to one more functional molecules, e.g., one or more peptides or proteins (e.g., one or more antibodies or ligands for a receptor) to generate a multi-specific molecule that binds to two or more different binding sites or target molecules. The presently disclosed anti-CD33 antibody or antigen-binding fragment thereof can in fact be derivatized or linked to more than one other functional molecules to generate multi-specific molecules that bind to more than two different binding sites and/or target molecules. To create a multi-specific molecule, a presently disclosed anti-CD33 antibody or an antigen-binding fragment thereof can be functionally linked (e.g., by chemical coupling, genetic fusion, noncovalent association or otherwise) to one or more other binding molecules, such as another antibody, antibody fragment, peptide or binding mimetic, such that a bispecific molecule.

In certain embodiments, the multi-specific molecule is a bispecific molecule. In certain embodiments, the bispecific molecules comprises at least a first binding specificity for CD33 and a second binding specificity for a second target epitope region. The second target epitope region can be a CD33 epitope, or a non-CD33 epitope, e.g., a different antigen. In certain embodiments, the multi-specific molecule comprises a first binding specificity for CD33, a second binding specificity for a second target, and a third binding specificity for a third target. In certain embodiments, the second target is an antigen expressed on the surface of an immune cell (e.g., a T cell, or a human immune effector cell). In certain embodiments, the multi-specific molecule is capable of recruiting the activity of that immune effector cell by specifically binding to the effector antigen on the human immune effector cell, thereby enhancing effector function. In certain embodiments, the third target is an antigen expressed on a senescent cell.

The multi-specific molecules of the presently disclosed subject matter can be prepared by conjugating the constituent binding specificities using methods known in the art. For example, each binding specificity of the multi-specific molecule can be generated separately and then conjugated to one another. When the binding specificities are proteins or peptides, a variety of coupling or cross-linking agents can be used for covalent conjugation. Non-limiting examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Conjugating agents can be SATA and sulfo-SMCC, both available from Pierce Chemical Co. (Rockford, IL).

When the binding specificities are antibodies, they can be conjugated via sulfhydryl bonding of the C-terminus hinge regions of the two heavy chains. In certain embodiments, the hinge region is modified to contain an odd number of sulfhydryl residues, preferably one, prior to conjugation.

Alternatively, both binding specificities can be encoded in the same vector and expressed and assembled in the same host cell. This method is particularly useful where the multi-specific molecule is a mAb x mAb, mAb x Fab, Fab x F(ab′)₂ or ligand x Fab fusion protein.

Binding of the multi-specific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography.

### 5.4. Nucleic Acids Encoding the Antibodies or Antigen-Binding Fragments and/or V_{H} and/or V_{L} Thereof

The presently disclosed subject matter provides nucleic acids encoding the anti-CD33 antibodies or antigen-binding fragments thereof disclosed herein. The presently disclosed subject matter provides nucleic acids encoding the heavy chain variable region sequence of any one of the presently disclosed anti-CD33 antibodies (e.g., 3-P14, 4-B2, 1-J19, 1-J19-2, 1-P13, 1-P23, 1-A20, 2-N3, 1-H19, 2-F18, and 4-P3 antibodies). In certain embodiments, the nucleic acid comprises or consists of a nucleotide sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the nucleotide sequence set forth in SEQ ID NO: 10, SEQ ID NO: 20, SEQ ID NO: 30, SEQ ID NO: 36, SEQ ID NO: 45, SEQ ID NO: 54, SEQ ID NO: 64, SEQ ID NO: 74, SEQ ID NO: 83, SEQ ID NO: 90, or SEQ ID NO: 93. In certain embodiments, the nucleic acid comprises or consists of the nucleotide sequence set forth in SEQ ID NO: 10, SEQ ID NO: 20, SEQ ID NO: 30, SEQ ID NO: 36, SEQ ID NO: 45, SEQ ID NO: 54, SEQ ID NO: 64, SEQ ID NO: 74, SEQ ID NO: 83, SEQ ID NO: 90, or SEQ ID NO: 93.

The presently disclosed subject matter provides nucleic acids encoding the light chain variable region sequence of any one of the presently disclosed anti-CD33 antibodies (e.g., 3-P14, 4-B2, 1-J19, 1-J19-2, 1-P13, 1-P23, 1-A20, 2-N3, 1-H19, 2-F18, and 4-P3 antibodies). In certain embodiments, the nucleic acid comprises or consists of a nucleotide sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the nucleotide sequence set forth in SEQ ID NO: 11, SEQ ID NO: 21, SEQ ID NO: 31, SEQ ID NO: 46, SEQ ID NO: 55, SEQ ID NO: 65, SEQ ID NO: 75, SEQ ID NO: 84, SEQ ID NO: 91, or SEQ ID NO: 94. In certain embodiments, the nucleic acid comprises or consists of the nucleotide sequence set forth in SEQ ID NO: 11, SEQ ID NO: 21, SEQ ID NO: 31, SEQ ID NO: 46, SEQ ID NO: 55, SEQ ID NO: 65, SEQ ID NO: 75, SEQ ID NO: 84, SEQ ID NO: 91, or SEQ ID NO: 94. Further provided are vectors comprising the presently disclosed nucleic acids. In certain embodiments, the vector is an expression vector. The presently disclosed subject matter further provides host cells comprising the vectors disclosed herein. In certain embodiments, the host cells are T cells.

### 5.5. Pharmaceutical Compositions and Methods of Treatment

The presently disclosed subject matter provides compositions comprising a presently disclosed anti-CD33 antibody or an antigen-binding fragment thereof, a presently disclosed immunoconjugate, a presently disclosed multi-specific molecule. In certain embodiments, the composition is a pharmaceutical composition further comprising a pharmaceutically acceptable carrier.

Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of the injection can, as is well known in the art, be formulated so as to provide quick, sustained or delayed release of the active ingredient after administration to the mammal.

The presently disclosed subject matter provides various methods of using the anti-CD33 antibodies or antigen-binding fragments thereof, the immunoconjugate, the multi-specific molecule, and the composition disclosed herein. For example, the presently disclosed subject matter provides methods for treating or ameliorating a disease or disorder in a subject. In certain embodiments, the method comprises administering one or more of the anti-CD33 antibodies or antigen-binding fragments thereof, the immunoconjugate, the multi-specific molecule, or the composition disclosed herein to the subject. In certain embodiments, the disease or disorder is associated with CD33. In certain embodiments, the disease or disorder is associated with overexpression of CD33. In certain embodiments, the disease or disorder is tumor.

The presently disclosed subject matter provides methods of reducing tumor burden in a subject. In certain embodiments, the method comprises administering one or more of the anti-CD33 antibodies or antigen-binding fragments thereof, the immunoconjugate, the multi-specific molecule, or the composition disclosed herein to the subject. The presently disclosed anti-CD33 antibodies or antigen-binding fragments thereof can reduce the number of tumor cells, reduce tumor size, and/or eradicate the tumor in the subject.

The presently disclosed subject matter also provides methods of increasing or lengthening survival of a subject having a tumor. In certain embodiments, the method comprises administering one or more of the anti-CD33 antibodies or antigen-binding fragments thereof, the immunoconjugate, the multi-specific molecule, or the composition disclosed herein to the subject. The method can reduce or eradicate tumor burden in the subject.

The presently disclosed subject matter further provides methods for treating and/or preventing a tumor in a subject. In certain embodiments, the method comprises administering one or more of the anti-CD33 antibodies or antigen-binding fragments thereof, the immunoconjugate, the multi-specific molecule, or the composition disclosed herein to the subject.

Such methods comprise administering the presently disclosed anti-CD33 antibodies or antigen-binding fragments thereof in an amount effective, a presently disclosed composition (e.g., a pharmaceutical composition) to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

In certain embodiments, the tumor is cancer. In certain embodiments, the tumor is selected from the group consisting of hematological cancer, and solid tissue cancer. In certain embodiments, the hematological cancer is selected from the group consisting of acute myeloid leukemia (AML), myelodysplastic syndromes (MDS), acute lymphoblastic leukemia (ALL), chronic lymphocytic leukemia (CLL), myeloproliferative neoplasms (MPNs), and chronic myeloid neoplasms. In certain embodiments, the tumor is acute myeloid leukemia AML.

Any suitable method or route can be used to administer a presently disclosed anti-CD33 antibody, and optionally, to co-administer antineoplastic agents. Routes of administration include, but are not limited to, oral, intravenous, intraperitoneal, subcutaneous, intramuscular, intranodal, intratumoral, intraosseous, intrathecal, pleural, intrapleural, topical, and direct administration. It should be emphasized, however, that the presently disclosed subject matter is not limited to any particular method or route of administration.

The presently disclosed anti-CD33 antibodies or antigen-binding fragments thereof can be administered as a conjugate, which binds specifically to the receptor and delivers a toxic, lethal payload following ligand-toxin internalization.

### 5.6. Diagnostic and Prognostic Methods

The presently disclosed anti-CD33 antibodies, antigen-binding fragments thereof, multi-specific molecules, and nucleic acids encode thereof can be used for diagnostic and prognostic applications as well as use as research tools for detection of CD33 in a biological sample, in a cell, a tissue, or a blood sample. The presently disclosed subject matter provides methods for detecting CD33 in a cell, a tissue, or a blood sample. In certain embodiments, the method comprises: contacting a cell, a tissue, or a blood sample with the antibody, antigen-binding fragment thereof, or multi-specific molecule disclosed herein, wherein the antibody, antigen-binding fragment thereof or multi-specific molecule comprises a detectable label; and determining the amount of the labeled antibody, antigen-binding fragment thereof, or multi-specific molecule bound to the cell, tissue, or blood sample by measuring the amount of detectable label associated with the cell or tissue, wherein the amount of bound antibody, antigen-binding fragment thereof, or multi-specific molecule indicates the amount of CD33 in the cell, tissue, or a blood sample. The cell or tissue can be any cell or tissue, including any normal, healthy, or cancerous cells and tissues. In certain embodiments, the blood sample is a peripheral blood sample.

The presently disclosed anti-CD33 antibodies or antigen-binding fragments thereof can be used in methods known in the art relating to the localization and/or quantitation of CD33 polypeptides (e.g., for use in measuring levels of the CD33 protein within appropriate physiological samples, for use in diagnostic methods, for use in imaging the polypeptide, and the like). The presently disclosed anti-CD33 antibodies or antigen-binding fragments thereof can be used to isolate a CD33 polypeptide by standard techniques, such as affinity chromatography or immunoprecipitation. The presently disclosed anti-CD33 antibodies or antigen-binding fragments thereof can facilitate the purification of natural immunoreactive

CD33 proteins from biological samples, e.g., mammalian sera or cells as well as recombinantly-produced immunoreactive CD33 proteins expressed in a host system. Moreover, anti-CD33 antibodies of the present technology can be used to detect an immunoreactive CD33 protein (e.g., in plasma, a cellular lysate or cell supernatant) in order to evaluate the abundance and pattern of expression of the immunoreactive polypeptide. The presently disclosed anti-CD33 antibodies or antigen-binding fragments thereof can be used diagnostically to monitor immunoreactive CD33 protein levels in tissue as part of a clinical testing procedure, e.g., to determine the efficacy of a given treatment regimen. As noted above, the detection can be facilitated by coupling (i.e., physically linking) the presently disclosed anti-CD33 antibodies or antigen-binding fragments thereof to a detectable substance.

An exemplary method for detecting the presence or absence of an immunoreactive CD33 protein in a biological sample comprises contacting a biological sample from a subject with a presently disclosed anti-CD33 antibody or an antigen-binding fragment thereof, wherein the presence of an immunoreactive CD33 protein is detected in the biological sample. Detection may be accomplished by means of a detectable label attached to the antibody.

The term “labeled” with regard to the anti-CD33 antibody or antigen-binding fragment thereof is intended to encompass direct labeling of the antibody by coupling (i.e., physically linking) a detectable substance to the antibody, as well as indirect labeling of the antibody by reactivity with another compound that is directly labeled, such as a secondary antibody. Examples of indirect labeling include detection of a primary antibody using a fluorescently-labeled secondary antibody and end-labeling of a DNA probe with biotin such that it can be detected with fluorescently-labeled streptavidin.

In certain embodiments, the presently disclosed anti-CD33 antibodies or antigen-binding fragments thereof are conjugated to one or more detectable labels. For such uses, the presently disclosed anti-CD33 antibodies or antigen-binding fragments thereof may be detectably labeled by covalent or non-covalent attachment of a chromogenic, enzymatic, radioisotopic, isotopic, fluorescent, toxic, chemiluminescent, nuclear magnetic resonance contrast agent or other label.

The presently disclosed detection methods can be used to detect an immunoreactive CD33 protein in a biological sample in vitro as well as in vivo. Non-limiting examples of in vitro techniques for detection of an immunoreactive CD33 protein include enzyme linked immunosorbent assays (ELISAs), Western blots, immunoprecipitations, radioimmunoassay, and immunofluorescence. Furthermore, in vivo techniques for detection of an immunoreactive CD33 protein include introducing into a subject a labeled anti-CD33 antibody or an antigen-binding fragment thereof. For example, the anti-CD33 antibody or antigen-binding fragment thereof can be labeled with a radioactive marker whose presence and location in a subject can be detected by standard imaging techniques. In certain embodiments, the biological sample comprises CD33 protein molecules from the test subject.

The presently disclosed anti-CD33 antibodies or antigen-binding fragments thereof can be used to assay immunoreactive CD33 protein levels in a biological sample (e.g., human plasma) using antibody-based techniques. For example, protein expression in tissues can be studied with classical immunohistological methods. Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes or other radioactive agent, such as iodine (¹²⁵I, ¹²¹I, ¹³¹I), carbon (¹⁴C), sulfur (³⁵S), tritium (³H), indium (¹¹¹In), and technetium (^{99m}Tc), and fluorescent labels, such as fluorescein, rhodamine, and green fluorescent protein (GFP), as well as biotin.

In addition to assaying immunoreactive CD33 protein levels in a biological sample, the presently disclosed anti-CD33 antibodies or antigen-binding fragments thereof may be used for in vivo imaging of CD33. Antibodies useful for this method include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which can be incorporated into the anti-CD33 antibodies by labeling of nutrients for the relevant scFv clone. The presently disclosed anti-CD33 antibodies or antigen-binding fragments thereof, which are labeled with an appropriate detectable imaging moiety (such as a radioisotope (e.g., ¹³¹I, ¹¹¹IN ^{99m}Tc, ¹⁸F, ⁸⁹Zr), a radio-opaque substance, or a material detectable by nuclear magnetic resonance) are introduced (e.g., parenterally, subcutaneously, or intraperitoneally) into the subject. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ^{99m}Tc. The labeled anti-CD33 antibody or antigen-binding fragment thereof then accumulates at the location of cells which contain the specific target polypeptide. For example, the labeled anti-CD33 antibodies or antigen-binding fragments thereof accumulate within the subject in cells and tissues in which the CD33 protein has localized.

Thus, the presently disclosed subject matter provides diagnostic methods of a medical condition. In certain embodiments, the method comprises: (a) assaying the expression of immunoreactive CD33 protein by measuring binding of a presently disclosed anti-CD33 antibody or an antigen-binding fragment thereof in cells or body fluid of an individual; and (b) comparing the amount of immunoreactive CD33 protein present in the sample with a standard reference, wherein an increase or decrease in immunoreactive CD33 protein levels compared to the standard is indicative of a medical condition.

Furthermore, the presently disclosed anti-CD33 antibodies or antigen-binding fragments thereof may be used to purify immunoreactive CD33 protein from a sample. In certain embodiments, the antibodies are immobilized on a solid support. Non-limiting examples of such solid supports include plastics such as polycarbonate, complex carbohydrates such as agarose and sepharose, acrylic resins and such as polyacrylamide and latex beads. Techniques for coupling antibodies to such solid supports are well known in the art.

The simplest method to bind the antigen to the antibody-support matrix is to collect the beads in a column and pass the antigen solution down the column. The efficiency of this method depends on the contact time between the immobilized antibody and the antigen, which can be extended by using low flow rates. The immobilized antibody captures the antigen as it flows past. Alternatively, an antigen can be contacted with the antibody-support matrix by mixing the antigen solution with the support (e.g., beads) and rotating the slurry, allowing maximum contact between the antigen and the immobilized antibody. After the binding reaction has been completed, the slurry is passed into a column for collection of the beads. The beads are washed using a suitable washing buffer and then the pure or substantially pure antigen is eluted.

An antibody or polypeptide of interest can be conjugated to a solid support, such as a bead. In addition, a first solid support such as a bead can also be conjugated, if desired, to a second solid support, which can be a second bead or other support, by any suitable means, including those disclosed herein for conjugation of a polypeptide to a support. Accordingly, any of the conjugation methods and means disclosed herein with reference to conjugation of a polypeptide to a solid support can also be applied for conjugation of a first support to a second support, where the first and second solid support can be the same or different.

Appropriate linkers, which can be cross-linking agents, for use for conjugating a polypeptide to a solid support include a variety of agents that can react with a functional group present on a surface of the support, or with the polypeptide, or both. Reagents useful as cross-linking agents include homo-bi-functional and, in particular, hetero-bi-functional reagents. Useful bi-functional cross-linking agents include, but are not limited to, N-SIAB, dimaleimide, DTNB, N-SATA, N-SPDP, SMCC and 6-HYNIC. A cross-linking agent can be selected to provide a selectively cleavable bond between a polypeptide and the solid support. For example, a photolabile cross-linker, such as 3-amino-(2-nitrophenyl)propionic acid can be employed as a means for cleaving a polypeptide from a solid support. (Brown et al., Mol. Divers, pp, 4-12 (1995); Rothschild et al., Nucl. Acids Res., 24:351-66 (1996); and U.S. Pat. No. 5,643,722). Other cross-linking reagents are well-known in the art. (Scc, e.g., Wong (1991), supra; and Hermanson (1996), supra).

An antibody or polypeptide can be immobilized on a solid support, such as a bead, through a covalent amide bond formed between a carboxyl group functionalized bead and the amino terminus of the polypeptide or, conversely, through a covalent amide bond formed between an amino group functionalized bead and the carboxyl terminus of the polypeptide. In addition, a bi-functional trityl linker can be attached to the support, e.g., to the 4-nitrophenyl active ester on a resin, such as a Wang resin, through an amino group or a carboxyl group on the resin via an amino resin. Using a bi-functional trityl approach, the solid support can require treatment with a volatile acid, such as formic acid or trifluoroacetic acid to ensure that the polypeptide is cleaved and can be removed. In such a case, the polypeptide can be deposited as a beadless patch at the bottom of a well of a solid support or on the flat surface of a solid support. After addition of a matrix solution, the polypeptide can be desorbed into a MS.

Hydrophobic trityl linkers can also be exploited as acid-labile linkers by using a volatile acid or an appropriate matrix solution, e.g., a matrix solution containing 3-HPA, to cleave an amino linked trityl group from the polypeptide. Acid lability can also be changed. For example, trityl, monomethoxytrityl, dimethoxytrityl or trimethoxytrityl can be changed to the appropriate p-substituted, or more acid-labile tritylamine derivatives, of the polypeptide, i.e., trityl ether and tritylamine bonds can be made to the polypeptide. Accordingly, a polypeptide can be removed from a hydrophobic linker, e.g., by disrupting the hydrophobic attraction or by cleaving tritylether or tritylamine bonds under acidic conditions, including, if desired, under typical MS conditions, where a matrix, such as 3-HPA acts as an acid.

Orthogonally cleavable linkers can also be useful for binding a first solid support, e.g., a bead to a second solid support, or for binding a polypeptide of interest to a solid support. Using such linkers, a first solid support, e.g., a bead, can be selectively cleaved from a second solid support, without cleaving the polypeptide from the support; the polypeptide then can be cleaved from the bead at a later time. For example, a disulfide linker, which can be cleaved using a reducing agent, such as DTT, can be employed to bind a bead to a second solid support, and an acid cleavable bi-functional trityl group could be used to immobilize a polypeptide to the support. As desired, the linkage of the polypeptide to the solid support can be cleaved first, e.g., leaving the linkage between the first and second support intact. Trityl linkers can provide a covalent or hydrophobic conjugation and, regardless of the nature of the conjugation, the trityl group is readily cleaved in acidic conditions.

For example, a bead can be bound to a second support through a linking group which can be selected to have a length and a chemical nature such that high density binding of the beads to the solid support, or high density binding of the polypeptides to the beads, is promoted. Such a linking group can have, e.g., “tree-like” structure, thereby providing a multiplicity of functional groups per attachment site on a solid support. Examples of such linking group; include polylysine, polyglutamic acid, penta-erythrol and tris-hydroxy-aminomethane.

Noncovalent Binding Association. An antibody or polypeptide can be conjugated to a solid support, or a first solid support can also be conjugated to a second solid support, through a noncovalent interaction. For example, a magnetic bead made of a ferromagnetic material, which is capable of being magnetized, can be attracted to a magnetic solid support, and can be released from the support by removal of the magnetic field. Alternatively, the solid support can be provided with an ionic or hydrophobic moiety, which can allow the interaction of an ionic or hydrophobic moiety, respectively, with a polypeptide, e.g., a polypeptide containing an attached trityl group or with a second solid support having hydrophobic character.

A solid support can also be provided with a member of a specific binding pair and, therefore, can be conjugated to a polypeptide or a second solid support containing a complementary binding moiety. For example, a bead coated with avidin or with streptavidin can be bound to a polypeptide having a biotin moiety incorporated therein, or to a second solid support coated with biotin or derivative of biotin, such as iminobiotin.

It should be recognized that any of the binding members disclosed herein or otherwise known in the art can be reversed. Thus, biotin, e.g., can be incorporated into either a polypeptide or a solid support and, conversely, avidin or other biotin binding moiety would be incorporated into the support or the polypeptide, respectively. Other specific binding pairs contemplated for use herein include, but are not limited to, hormones and their receptors, enzyme, and their substrates, a nucleotide sequence and its complementary sequence, an antibody and the antigen to which it interacts specifically, and other such pairs knows to those skilled in the art.

The presently disclosed anti-CD33 antibodies or antigen-binding fragments thereof are useful in diagnostic methods. As such, the presently disclosed subject matter provides methods using the presently disclosed anti-CD33 antibodies or antigen-binding fragments thereof in diagnosis of CD33 activity in a subject. The presently disclosed anti-CD33 antibodies or antigen-binding fragments thereof may be selected such that they have any level of epitope binding specificity and high binding affinity to a CD33 polypeptide.

The presently disclosed anti-CD33 antibodies or antigen-binding fragments thereof can be used to detect an immunoreactive CD33 protein in a variety of standard assay formats. Such formats include immunoprecipitation, Western blotting, ELISA, radioimmunoassay, and immunometric assays. Biological samples can be obtained from any tissue or body fluid of a subject. In certain embodiments, the subject is at an early stage of cancer. In certain embodiments, the early stage of cancer is determined by the level or expression pattern of CD33 protein in a sample obtained from the subject. In certain embodiments, the sample is selected from the group consisting of urine, blood, serum, plasma, saliva, amniotic fluid, cerebrospinal fluid (CSF), and biopsied body tissue.

Immunometric or sandwich assays are one format for the diagnostic methods of the present technology. Such assays use one antibody, e.g., the anti-CD33 antibody or a population of anti-CD33 antibodies immobilized to a solid phase, and another anti-CD33 antibody or a population of anti-CD33 antibodies in solution. Typically, the solution anti-CD33 antibody or population of anti-CD33 antibodies is labeled. If an antibody population is used, the population can contain antibodies binding to different epitope specificities within the target polypeptide. Accordingly, the same population can be used for both solid phase and solution antibody. If anti-CD33 monoclonal antibodies are used, first and second CD33 monoclonal antibodies having different binding specificities are used for the solid and solution phase. Solid phase (also referred to as “capture”) and solution (also referred to as “detection”) antibodies can be contacted with target antigen in either order or simultaneously. If the solid phase antibody is contacted first, the assay is referred to as being a forward assay. Conversely, if the solution antibody is contacted first, the assay is referred to as being a reverse assay. If the target is contacted with both antibodies simultaneously, the assay is referred to as a simultaneous assay. After contacting the CD33 protein with the anti-CD33 antibody, a sample is incubated for a period that usually varies from about 10 min to about 24 hr and is usually about 1 hr. A wash step is then performed to remove components of the sample not specifically bound to the anti-CD33 antibody being used as a diagnostic reagent. When solid phase and solution antibodies are bound in separate steps, a wash can be performed after either or both binding steps. After washing, binding is quantified, typically by detecting a label linked to the solid phase through binding of labeled solution antibody. Usually for a given pair of antibodies or populations of antibodies and given reaction conditions, a calibration curve is prepared from samples containing known concentrations of target antigen. Concentrations of the immunoreactive CD33 protein in samples being tested are then read by interpolation from the calibration curve (i.e., standard curve). Analyte can be measured either from the amount of labeled solution antibody bound at equilibrium or by kinetic measurements of bound labeled solution antibody at a series of time points before equilibrium is reached. The slope of such a curve is a measure of the concentration of the CD33 protein in a sample.

Suitable supports for use in the above methods include, e.g., nitrocellulose membranes, nylon membranes, and derivatized nylon membranes, and also particles, such as agarose, a dextran-based gel, dipsticks, particulates, microspheres, magnetic particles, test tubes, microtiter wells, SEPHADEX™ (Amersham Pharmacia Biotech, Piscataway N.J.), and the like. Immobilization can be by absorption or by covalent attachment. Optionally, anti-CD33 antibodies can be joined to a linker molecule, such as biotin for attachment to a surface bound linker, such as avidin.

In certain embodiments, the presently disclosed anti-CD33 antibody or antigen-binding fragment thereof is conjugated to a diagnostic agent. The diagnostic agent may comprise a radioactive or non-radioactive label, a contrast agent (such as for magnetic resonance imaging, computed tomography or ultrasound), and the radioactive label can be a gamma-, beta-, alpha-, Auger electron-, or positron-emitting isotope. A diagnostic agent is a molecule which is administered conjugated to an antibody moiety, i.e., antibody or antibody fragment, or subfragment, and is useful in diagnosing or detecting a disease by locating the cells comprising the antigen.

Useful diagnostic agents include, but are not limited to, radioisotopes, dyes (such as with the biotin-streptavidin complex), contrast agents, fluorescent compounds or molecules and enhancing agents (e.g., paramagnetic ions) for magnetic resonance imaging (MRI). In certain embodiments, the diagnostic agents are selected from the group consisting of radioisotopes, enhancing agents for use in magnetic resonance imaging, and fluorescent compounds. Chelates may be coupled to the presently disclosed anti-CD33 antibodies or antigen-binding fragments thereof using standard chemistries. The chelate is normally linked to the antibody by a group which enables formation of a bond to the molecule with minimal loss of immunoreactivity and minimal aggregation and/or internal cross-linking.

### 5.7. Kits

The presently disclosed subject matter provides kits for treatment or ameliorating a disease or disorder associated with CD33 (e.g., AML), and/or detecting CD33. In certain embodiments, the kit comprises the anti-CD33 antibodies or antigen-binding fragments thereof, the immunoconjugate, the multi-specific molecule, or the composition disclosed herein. In certain embodiments, the kit comprises a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In certain embodiments, the kit further comprises instructions for administering the anti-CD33 antibodies or antigen-binding fragments thereof, the immunoconjugate, the multi-specific molecule, or the composition disclosed herein to a subject in need the treatment. The instructions can generally include information about the use of the anti-CD33 antibodies or antigen-binding fragments thereof, the immunoconjugate, the multi-specific molecule, and the composition disclosed herein for the treatment or ameliorating a disease or disorder. In certain embodiments, the instructions include at least one of the following: description of the therapeutic agent; dosage schedule and administration for treatment and/or prevention of a tumor or neoplasm or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### 5.8. Exemplary Embodiments

A1. In certain non-limiting embodiments, the presently disclosed subject matter provides an anti-CD33 antibody or an antigen-binding fragment thereof, comprising a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, SEQ ID NO: 18, SEQ ID NO: 28, SEQ ID NO: 35, SEQ ID NO: 43, SEQ ID NO: 52, SEQ ID NO: 62, SEQ ID NO: 72, SEQ ID NO: 81, or SEQ ID NO: 88.

A2. In certain non-limiting embodiments, the presently disclosed subject matter provides an anti-CD33 antibody or an antigen-binding fragment thereof, comprising a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, SEQ ID NO: 19, SEQ ID NO: 29, SEQ ID NO: 44, SEQ ID NO: 53, SEQ ID NO: 63, SEQ ID NO: 73, SEQ ID NO: 82, SEQ ID NO: 89, or SEQ ID NO: 92.

A3. In certain non-limiting embodiments, the presently disclosed subject matter provides an anti-CD33 antibody or an antigen-binding fragment thereof, comprising
(a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, SEQ ID NO: 18, SEQ ID NO: 28, SEQ ID NO: 35, SEQ ID NO: 43, SEQ ID NO: 52, SEQ ID NO: 62, SEQ ID NO: 72, SEQ ID NO: 81, or SEQ ID NO: 88; and
(b) a light chain variable region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 9, SEQ ID NO: 19, SEQ ID NO: 29, SEQ ID NO: 44, SEQ ID NO: 53, SEQ ID NO: 63, SEQ ID NO: 73, SEQ ID NO: 82, SEQ ID NO: 89, or SEQ ID NO: 92.

A4. In certain non-limiting embodiments, the presently disclosed subject matter provides an anti-CD33 antibody or an antigen-binding fragment thereof, comprising a heavy chain variable region and a light chain variable region, wherein the heavy chain variable region and the light chain variable region are selected from the group consisting of:
(a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9;
(b) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 18, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 19;
(c) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 28, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 29;
(d) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 35, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 29;
(e) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 43, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 44;
(f) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 52, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 53;
(g) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 62, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 63;
(h) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 72, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 73;
(i) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 81, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 81;
(j) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 88, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 89; and
(k) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 92.

A5. In certain non-limiting embodiments, the presently disclosed subject matter provides an anti-CD33 antibody or an antigen-binding fragment thereof, comprising a heavy chain variable region comprising the amino acid sequence set forth in SEQ ID NO: 8, SEQ ID NO: 18, SEQ ID NO: 28, SEQ ID NO: 35, SEQ ID NO: 43, SEQ ID NO: 52, SEQ ID NO: 62, SEQ ID NO: 72, SEQ ID NO: 81, or SEQ ID NO: 88.

A6. In certain non-limiting embodiments, the presently disclosed subject matter provides an anti-CD33 antibody or an antigen-binding fragment thereof, comprising a light chain variable region comprising the amino acid sequence set forth in SEQ ID NO: 9, SEQ ID NO: 19, SEQ ID NO: 29, SEQ ID NO: 44, SEQ ID NO: 53, SEQ ID NO: 63, SEQ ID NO: 73, SEQ ID NO: 82, SEQ ID NO: 89, or SEQ ID NO: 92.

A7. In certain non-limiting embodiments, the presently disclosed subject matter provides an anti-CD33 antibody or an antigen-binding fragment thereof, comprising
(a) a heavy chain variable region comprising the amino acid sequence set forth in SEQ ID SEQ ID NO: 8, SEQ ID NO: 18, SEQ ID NO: 28, SEQ ID NO: 35, SEQ ID NO: 43, SEQ ID NO: 52, SEQ ID NO: 62, SEQ ID NO: 72, SEQ ID NO: 81, or SEQ ID NO: 88; and
(b) a light chain variable region comprising the amino acid sequence set forth in SEQ ID NO: 9, SEQ ID NO: 19, SEQ ID NO: 29, SEQ ID NO: 44, SEQ ID NO: 53, SEQ ID NO: 63, SEQ ID NO: 73, SEQ ID NO: 82, SEQ ID NO: 89, or SEQ ID NO: 92.

A8. The foregoing antibody or antigen-binding fragment thereof of any one of A1-A7, wherein
(a) the heavy chain variable region comprises the amino acid sequence set forth in SEQ ID NO: 8, and the light chain variable region comprises the amino acid sequence set forth in SEQ ID NO: 9;
(b) the heavy chain variable region comprises the amino acid sequence set forth in SEQ ID NO: 18, and the light chain variable region comprises the amino acid sequence set forth in SEQ ID NO: 19;
(c) the heavy chain variable region comprises the amino acid sequence set forth in SEQ ID NO: 28, and the light chain variable region comprises the amino acid sequence set forth in SEQ ID NO: 29;
(d) the heavy chain variable region comprises the amino acid sequence set forth in SEQ ID NO: 35, and the light chain variable region comprises the amino acid sequence set forth in SEQ ID NO: 29;
(e) the heavy chain variable region comprises the amino acid sequence set forth in SEQ ID NO: 43, and the light chain variable region comprises the amino acid sequence set forth in SEQ ID NO: 44;
(f) the heavy chain variable region comprises the amino acid sequence set forth in SEQ ID NO: 52, and the light chain variable region comprises the amino acid sequence set forth in SEQ ID NO: 53;
(g) the heavy chain variable region comprises the amino acid sequence set forth in SEQ ID NO: 62, and the light chain variable region comprises the amino acid sequence set forth in SEQ ID NO: 63;
(h) the heavy chain variable region comprises the amino acid sequence set forth in SEQ ID NO: 72, and the light chain variable region comprises the amino acid sequence set forth in SEQ ID NO: 73;
(i) the heavy chain variable region comprises the amino acid sequence set forth in SEQ ID NO: 81, and the light chain variable region comprises the amino acid sequence set forth in SEQ ID NO: 82;
(j) the heavy chain variable region comprises the amino acid sequence set forth in SEQ ID NO: 88, and the light chain variable region comprises the amino acid sequence set forth in SEQ ID NO: 89; or
(k) the heavy chain variable region comprises the amino acid sequence set forth in SEQ ID NO: 98, and the light chain variable region comprises the amino acid sequence set forth in SEQ ID NO: 92.

A9. The foregoing anti-CD33 antibody or an antigen-binding fragment thereof of A8, wherein:
(a) the heavy chain variable region comprises the amino acid sequence set forth in SEQ ID NO: 8, and the light chain variable region comprises the amino acid sequence set forth in SEQ ID NO: 9;
(b) the heavy chain variable region comprises the amino acid sequence set forth in SEQ ID NO: 18, and the light chain variable region comprises the amino acid sequence set forth in SEQ ID NO: 19; or
(c) the heavy chain variable region comprises the amino acid sequence set forth in SEQ ID NO: 28, and the light chain variable region comprises the amino acid sequence set forth in SEQ ID NO: 29.

A10. In certain non-limiting embodiments, the presently disclosed subject matter provides an anti-CD33 antibody or an antigen-binding fragment thereof, comprising a heavy chain variable region that comprises CDR1, CDR2, and CDR3 domains; and a light chain variable region that comprises CDR1, CDR2, and CDR3 domains, wherein the heavy chain variable region and light chain variable region CDR3 domains are selected from the group consisting of:
(a) a heavy chain variable region CDR3 comprising the amino acid sequence set forth in SEQ ID NO: 4 or a conservative modification thereof; and a light chain variable region CDR3 comprising the amino acid sequence set forth in SEQ ID NO: 7 or a conservative modification thereof;
(b) a heavy chain variable region CDR3 comprising the amino acid sequence set forth in SEQ ID NO: 14 or a conservative modification thereof; and a light chain variable region CDR3 comprising the amino acid sequence set forth in SEQ ID NO: 17 or a conservative modification thereof;
(c) a heavy chain variable region CDR3 comprising the amino acid sequence set forth in SEQ ID NO: 24 or a conservative modification thereof; and a light chain variable region CDR3 comprising the amino acid sequence set forth in SEQ ID NO: 27 or a conservative modification thereof;
(d) a heavy chain variable region CDR3 comprising the amino acid sequence set forth in SEQ ID NO: 34 or a conservative modification thereof; and a light chain variable region CDR3 comprising the amino acid sequence set forth in SEQ ID NO: 27 or a conservative modification thereof;
(e) a heavy chain variable region CDR3 comprising the amino acid sequence set forth in SEQ ID NO: 39 or a conservative modification thereof; and a light chain variable region CDR3 comprising the amino acid sequence set forth in SEQ ID NO: 42 or a conservative modification thereof;
(f) a heavy chain variable region CDR3 comprising the amino acid sequence set forth in SEQ ID NO: 48 or a conservative modification thereof; and a light chain variable region CDR3 comprising the amino acid sequence set forth in SEQ ID NO: 51 or a conservative modification thereof;
(g) a heavy chain variable region CDR3 comprising the amino acid sequence set forth in SEQ ID NO: 58 or a conservative modification thereof; and a light chain variable region CDR3 comprising the amino acid sequence set forth in SEQ ID NO: 61 or a conservative modification thereof;
(h) a heavy chain variable region CDR3 comprising the amino acid sequence set forth in SEQ ID NO: 68 or a conservative modification thereof; and a light chain variable region CDR3 comprising the amino acid sequence set forth in SEQ ID NO: 71 or a conservative modification thereof;
(i) a heavy chain variable region CDR3 comprising the amino acid sequence set forth in SEQ ID NO: 77 or a conservative modification thereof; and a light chain variable region CDR3 comprising the amino acid sequence set forth in SEQ ID NO: 80 or a conservative modification thereof; or
(j) a heavy chain variable region CDR3 comprising the amino acid sequence set forth in SEQ ID NO: 87 or a conservative modification thereof; and a light chain variable region CDR3 comprising the amino acid sequence set forth in SEQ ID NO: 42 or a conservative modification thereof.

A11. The foregoing antibody or antigen-binding fragment thereof of A10, wherein the heavy chain variable region and light chain variable region CDR2 domains are selected from:
(a) a heavy chain variable region CDR2 comprising the amino acid sequence set forth in SEQ ID NO: 3 or a conservative modification thereof; and a light chain variable region CDR2 comprising the amino acid sequence set forth in SEQ ID NO: 6 or a conservative modification thereof;
(b) a heavy chain variable region CDR2 comprising the amino acid sequence set forth in SEQ ID NO: 13 or a conservative modification thereof; and a light chain variable region CDR2 comprising the amino acid sequence set forth in SEQ ID NO: 16 or a conservative modification thereof;
(c) a heavy chain variable region CDR2 comprising the amino acid sequence set forth in SEQ ID NO: 23 or a conservative modification thereof; and a light chain variable region CDR2 comprising the amino acid sequence set forth in SEQ ID NO: 26 or a conservative modification thereof;
(d) a heavy chain variable region CDR2 comprising the amino acid sequence set forth in SEQ ID NO: 33 or a conservative modification thereof; and a light chain variable region CDR2 comprising the amino acid sequence set forth in SEQ ID NO: 26 or a conservative modification thereof;
(e) a heavy chain variable region CDR2 comprising the amino acid sequence set forth in SEQ ID NO: 38 or a conservative modification thereof; and a light chain variable region CDR2 comprising the amino acid sequence set forth in SEQ ID NO: 41 or a conservative modification thereof;
(f) a heavy chain variable region CDR2 comprising the amino acid sequence set forth in SEQ ID NO: 38 or a conservative modification thereof; and a light chain variable region CDR2 comprising the amino acid sequence set forth in SEQ ID NO: 50 or a conservative modification thereof;
(g) a heavy chain variable region CDR2 comprising the amino acid sequence set forth in SEQ ID NO: 57 or a conservative modification thereof; and a light chain variable region CDR2 comprising the amino acid sequence set forth in SEQ ID NO: 60 or a conservative modification thereof;
(h) a heavy chain variable region CDR2 comprising the amino acid sequence set forth in SEQ ID NO: 67 or a conservative modification thereof; and a light chain variable region CDR2 comprising the amino acid sequence set forth in SEQ ID NO: 70 or a conservative modification thereof;
(i) a heavy chain variable region CDR2 comprising the amino acid sequence set forth in SEQ ID NO: 57 or a conservative modification thereof; and a light chain variable region CDR2 comprising the amino acid sequence set forth in SEQ ID NO: 79 or a conservative modification thereof; and
(j) a heavy chain variable region CDR2 comprising the amino acid sequence set forth in SEQ ID NO: 86 or a conservative modification thereof; and a light chain variable region CDR2 comprising the amino acid sequence set forth in SEQ ID NO: 41 or a conservative modification thereof.

A12. The foregoing antibody or antigen-binding fragment thereof of A10 or All, wherein the heavy chain variable region and light chain variable region CDR1 domains are selected from the group consisting of:
(a) a heavy chain variable region CDR1 comprising the amino acid sequence set forth in SEQ ID NO: 2 or a conservative modification thereof; and a light chain variable region CDR1 comprising the amino acid sequence set forth in SEQ ID NO: 5 or a conservative modification thereof;
(b) a heavy chain variable region CDR1 comprising the amino acid sequence set forth in SEQ ID NO: 12 or a conservative modification thereof; and a light chain variable region CDR1 comprising the amino acid sequence set forth in SEQ ID NO: 15 or a conservative modification thereof;
(c) a heavy chain variable region CDR1 comprising the amino acid sequence set forth in SEQ ID NO: 22 or a conservative modification thereof; and a light chain variable region CDR1 comprising the amino acid sequence set forth in SEQ ID NO: 25 or a conservative modification thereof;
(d) a heavy chain variable region CDR1 comprising the amino acid sequence set forth in SEQ ID NO: 32 or a conservative modification thereof; and a light chain variable region CDR1 comprising the amino acid sequence set forth in SEQ ID NO: 25 or a conservative modification thereof;
(c) a heavy chain variable region CDR1 comprising the amino acid sequence set forth in SEQ ID NO: 37 or a conservative modification thereof; and a light chain variable region CDR1 comprising the amino acid sequence set forth in SEQ ID NO: 40 or a conservative modification thereof;
(f) a heavy chain variable region CDR1 comprising the amino acid sequence set forth in SEQ ID NO: 47 or a conservative modification thereof; and a light chain variable region CDR1 comprising the amino acid sequence set forth in SEQ ID NO: 49 or a conservative modification thereof;
(g) a heavy chain variable region CDR1 comprising the amino acid sequence set forth in SEQ ID NO: 56 or a conservative modification thereof; and a light chain variable region CDR1 comprising the amino acid sequence set forth in SEQ ID NO: 59 or a conservative modification thereof;
(h) a heavy chain variable region CDR1 comprising the amino acid sequence set forth in SEQ ID NO: 66 or a conservative modification thereof; and a light chain variable region CDR1 comprising the amino acid sequence set forth in SEQ ID NO: 69 or a conservative modification thereof;
(i) a heavy chain variable region CDR1 comprising the amino acid sequence set forth in SEQ ID NO: 76 or a conservative modification thereof; and a light chain variable region CDR1 comprising the amino acid sequence set forth in SEQ ID NO: 78 or a conservative modification thereof; or
(j) a heavy chain variable region CDR1 comprising the amino acid sequence set forth in SEQ ID NO: 86 or a conservative modification thereof; and a light chain variable region CDR1 comprising the amino acid sequence set forth in SEQ ID NO: 5 or a conservative modification thereof.

A13. The foregoing antibody or antigen-binding fragment thereof of any one of A10-A12, wherein one or more of the CDR sequences have up to about 5 amino acid substitutions.

A14. The foregoing antibody or antigen-binding fragment thereof of any one of A10-A13, wherein one or more of the CDR sequences have up to about 3 amino acid substitutions.

A15. In certain non-limiting embodiments, the presently disclosed subject matter provides an anti-CD33 antibody or an antigen-binding fragment thereof, comprising:
(a) a heavy chain variable region CDR1 comprising a comprising the amino acid sequence set forth in SEQ ID NO: 2, a CDR2 comprising the amino acid sequence set forth in SEQ ID NO: 3, and a CDR3 comprising the amino acid sequence set forth in SEQ ID NO: 4;
(b) a heavy chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14;
(c) a heavy chain variable region comprising a CDR1 comprising the amino acid sequence set forth in SEQ ID NO: 22, a CDR2 comprising the amino acid sequence set forth in SEQ ID NO: 23, and a CDR3 comprising the amino acid sequence set forth in SEQ ID NO: 24;
(d) a heavy chain variable region comprising a CDR1 comprising the amino acid sequence set forth in SEQ ID NO: 32, a CDR2 comprising the amino acid sequence set forth in SEQ ID NO: 33, and a CDR3 comprising the amino acid sequence set forth in SEQ ID NO: 34;
(e) a heavy chain variable region comprising a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39;
(f) a heavy chain variable region comprising a CDR1 comprising the amino acid sequence set forth in SEQ ID NO: 47, a CDR2 comprising the amino acid sequence set forth in SEQ ID NO: 38, and a CDR3 comprising the amino acid sequence set forth in SEQ ID NO: 48;
(g) a heavy chain variable region comprising a CDR1 comprising the amino acid sequence set forth in SEQ ID NO: 56, a CDR2 comprising the amino acid sequence set forth in SEQ ID NO:57, and a CDR3 comprising the amino acid sequence set forth in SEQ ID NO: 58;
(h) a heavy chain variable region comprising a CDR1 comprising the amino acid sequence set forth in SEQ ID NO: 66, a CDR2 comprising the amino acid sequence set forth in SEQ ID NO: 67, and a CDR3 comprising the amino acid sequence set forth in SEQ ID NO: 68;
(i) a heavy chain variable region comprising a CDR1 comprising the amino acid sequence set forth in SEQ ID NO: 76, a CDR2 comprising the amino acid sequence set forth in SEQ ID NO: 57, and a CDR3 comprising the amino acid sequence set forth in SEQ ID NO: 77; or
(j) a heavy chain variable region comprising a CDR1 comprising the amino acid sequence set forth in SEQ ID NO: 85, a CDR2 comprising the amino acid sequence set forth in SEQ ID NO: 86, and a CDR3 comprising the amino acid sequence set forth in SEQ ID NO: 87.

A16. In certain non-limiting embodiments, the presently disclosed subject matter provides an anti-CD33 antibody or an antigen-binding fragment thereof, comprising:
(a) a light chain variable region comprising a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7;
(b) a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17;
(c) a light chain variable region comprising a CDR1 comprising the amino acid sequence set forth in SEQ ID NO: 25, a CDR2 comprising the amino acid sequence set forth in SEQ ID NO: 26, and a CDR3 comprising the amino acid sequence set forth in SEQ ID NO: 27;
(d) a light chain variable region comprising a CDR1 comprising the amino acid sequence set forth in SEQ ID NO: 40, a CDR2 comprising the amino acid sequence set forth in SEQ ID NO: 41, and a CDR3 comprising the amino acid sequence set forth in SEQ ID NO: 42;
(e) a light chain variable region comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51;
(f) a light chain variable region comprising a CDR1 comprising the amino acid sequence set forth in SEQ ID NO: 59, a CDR2 comprising the amino acid sequence set forth in SEQ ID NO: 60, and a CDR3 comprising the amino acid sequence set forth in SEQ ID NO: 61;
(g) a light chain variable region comprising a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71;
(h) a light chain variable region comprising a CDR1 comprising the amino acid sequence set forth in SEQ ID NO: 78, a CDR2 comprising the amino acid sequence set forth in SEQ ID NO: 79, and a CDR3 comprising the amino acid sequence set forth in SEQ ID NO: 80; or
(i) a light chain variable region comprising a CDR1 comprising the amino acid sequence set forth in SEQ ID NO: 5, a CDR2 comprising the amino acid sequence set forth in SEQ ID NO: 41, and a CDR3 comprising the amino acid sequence set forth in SEQ ID NO: 42.

A17. In certain non-limiting embodiments, the presently disclosed subject matter provides an anti-CD33 antibody or an antigen-binding fragment thereof, comprising:
(a) a heavy chain variable region comprising a CDR1 comprising the amino acid sequence set forth in SEQ ID NO: 2, a CDR2 comprising the amino acid sequence set forth in SEQ ID NO: 3, and a CDR3 comprising the amino acid sequence set forth in SEQ ID NO: 4; and a light chain variable region comprising a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7;
(b) a heavy chain variable region comprising a CDR1 comprising the amino acid sequence set forth in SEQ ID NO: 12, a CDR2 comprising an amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17;
(c) a heavy chain variable region comprising a CDR1 comprising the amino acid sequence set forth in SEQ ID NO: 22, a CDR2 comprising the amino acid sequence set forth in SEQ ID NO: 23, and a CDR3 comprising the amino acid sequence set forth in SEQ ID NO: 24; and a light chain variable region comprising a CDR1 comprising the amino acid sequence set forth in SEQ ID NO: 25, a CDR2 comprising the amino acid sequence set forth in SEQ ID NO: 26, and a CDR3 comprising the amino acid sequence set forth in SEQ ID NO: 27;
(d) a heavy chain variable region comprising a CDR1 comprising the amino acid sequence set forth in SEQ ID NO: 32, a CDR2 comprising the amino acid sequence set forth in SEQ ID NO: 33, and a CDR3 comprising the amino acid sequence set forth in SEQ ID NO: 34; and a light chain variable region comprising a CDR1 comprising the amino acid sequence set forth in SEQ ID NO: 25, a CDR2 comprising the amino acid sequence set forth in SEQ ID NO: 26, and a CDR3 comprising the amino acid sequence set forth in SEQ ID NO: 27;
(e) a heavy chain variable region comprising a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39; and a light chain variable region comprising a CDR1 comprising the amino acid sequence set forth in SEQ ID NO: 40, a CDR2 comprising the amino acid sequence set forth in SEQ ID NO: 41, and a CDR3 comprising the amino acid sequence set forth in SEQ ID NO: 42;
(f) a heavy chain variable region comprising a CDR1 comprising the amino acid sequence set forth in SEQ ID NO: 47, a CDR2 comprising the amino acid sequence set forth in SEQ ID NO: 38, and a CDR3 comprising the amino acid sequence set forth in SEQ ID NO: 48; and a light chain variable region comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51;
(g) a heavy chain variable region comprising a CDR1 comprising the amino acid sequence set forth in SEQ ID NO: 56, a CDR2 comprising the amino acid sequence set forth in SEQ ID NO: 57, and a CDR3 comprising the amino acid sequence set forth in SEQ ID NO: 58; and a light chain variable region comprising a CDR1 comprising the amino acid sequence set forth in SEQ ID NO: 59, a CDR2 comprising the amino acid sequence set forth in SEQ ID NO: 60, and a CDR3 comprising the amino acid sequence set forth in SEQ ID NO: 61;
(h) a heavy chain variable region comprising a CDR1 comprising the amino acid sequence set forth in SEQ ID NO: 66, a CDR2 comprising the amino acid sequence set forth in SEQ ID NO: 67, and a CDR3 comprising the amino acid sequence set forth in SEQ ID NO: 68; and a light chain variable region comprising a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71;
(i) a heavy chain variable region comprising a CDR1 comprising the amino acid sequence set forth in SEQ ID NO: 76, a CDR2 comprising the amino acid sequence set forth in SEQ ID NO: 57, and a CDR3 comprising the amino acid sequence set forth in SEQ ID NO: 77; and a light chain variable region comprising a CDR1 comprising the amino acid sequence set forth in SEQ ID NO: 78, a CDR2 comprising the amino acid sequence set forth in SEQ ID NO: 79, and a CDR3 comprising the amino acid sequence set forth in SEQ ID NO: 80; or
(j) a heavy chain variable region comprising a CDR1 comprising the amino acid sequence set forth in SEQ ID NO: 85, a CDR2 comprising the amino acid sequence set forth in SEQ ID NO: 86, and a CDR3 comprising the amino acid sequence set forth in SEQ ID NO: 87; and a light chain variable region comprising a CDR1 comprising the amino acid sequence set forth in SEQ ID NO: 5, a CDR2 comprising the amino acid sequence set forth in SEQ ID NO: 41, and a CDR3 comprising the amino acid sequence set forth in SEQ ID NO: 42.

A18. The foregoing antibody or antigen-binding fragment thereof of A17, wherein:
(a) a heavy chain variable region comprising a CDR1 comprising the amino acid sequence set forth in SEQ ID NO: 2, a CDR2 comprising the amino acid sequence set forth in SEQ ID NO: 3, and a CDR3 comprising the amino acid sequence set forth in SEQ ID NO: 4; and a light chain variable region comprising a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7;
(b) a heavy chain variable region comprising a CDR1 comprising the amino acid sequence set forth in SEQ ID NO: 12, a CDR2 comprising an amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17; or
(c) a heavy chain variable region comprising a CDR1 comprising the amino acid sequence set forth in SEQ ID NO: 22, a CDR2 comprising the amino acid sequence set forth in SEQ ID NO: 23, and a CDR3 comprising the amino acid sequence set forth in SEQ ID NO: 24; and a light chain variable region comprising a CDR1 comprising the amino acid sequence set forth in SEQ ID NO: 25, a CDR2 comprising the amino acid sequence set forth in SEQ ID NO: 26, and a CDR3 comprising the amino acid sequence set forth in SEQ ID NO: 27.

A19. The foregoing antibody or antigen-binding fragment thereof of any one of A1-A18, wherein the antibody comprises a comprises a heavy chain constant region and/or a light chain constant region.

A20. The foregoing antibody or antigen-binding fragment thereof of A19, wherein:
(a) the heavy chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 95; and/or
(b) the light chain constant region comprises an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 96.

A21. The foregoing antibody or antigen-binding fragment thereof of A19 or A20, wherein:
(a) the heavy chain constant region comprises the amino acid sequence set forth in SEQ ID NO: 95; and/or
(b) the light chain constant region comprises the amino acid sequence set forth in SEQ ID NO: 96 or SEQ ID NO: 99.

A22. The foregoing antibody or antigen-binding fragment thereof of any one of A1-A21, wherein the antibody or an antigen-binding fragment thereof comprises:
(a) a heavy chain comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 97, SEQ ID NO: 100, or SEQ ID NO: 102; and/or
(b) a light chain comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 98, SEQ ID NO: 101, or SEQ ID NO: 103.

A23. The foregoing antibody or antigen-binding fragment thereof of any one of A1-A21, wherein the antibody or an antigen-binding fragment thereof comprises:
(a) a heavy chain comprising the amino acid sequence set forth in SEQ ID NO: 97, SEQ ID NO: 100, or SEQ ID NO: 102; and/or
(b) a light chain comprising the amino acid sequence set forth in SEQ ID NO: 98, SEQ ID NO: 101, or SEQ ID NO: 103.

A24. The foregoing antibody or antigen-binding fragment thereof of any one of A1-A23, wherein the antibody or antigen-binding fragment thereof binds to a CD33 comprising the amino acid sequence set forth in SEQ ID NO: 215 or a fragment thereof.

A25. The foregoing antibody or antigen-binding fragment thereof of any one of A1-A24, wherein the antibody comprises a human variable region framework region.

A26. The foregoing antibody or antigen-binding fragment thereof of any one of A1-A25, which is a fully human or an antigen-binding fragment thereof.

A27. The foregoing antibody or antigen-binding fragment thereof of any one of A1-A24, which is a chimeric antibody or an antigen-binding fragment thereof.

A28. The foregoing antibody or antigen-binding fragment thereof of any one of A1-A24, which is a humanized antibody or an antigen-binding fragment thereof.

A29. The foregoing antibody or antigen-binding fragment thereof of any one of A1-A28, wherein the antigen-binding fragment is a Fab, Fab′, F(ab′)2, variable fragment (Fv), or single chain variable region (scFv).

A30. The foregoing antibody or antigen-binding fragment thereof of A29, wherein the antigen antigen-binding fragment is an scFv.

A31. In certain non-limiting embodiments, the presently disclosed subject matter provides an antibody or an antigen-binding fragment thereof, which cross-competes for binding to CD33 with an antibody or an antigen-binding fragment thereof of any one of A1-A30.

A32. In certain non-limiting embodiments, the presently disclosed subject matter provides an antibody or an antigen-binding fragment thereof, which binds to the same epitope region on CD33 with an antibody or an antigen-binding fragment thereof of any one of claims A1-A30.

B1. In certain non-limiting embodiments, the presently disclosed subject matter provides a composition comprising the antibody or antigen-binding fragment thereof of any one of A1-A32.

B2. The foregoing composition of B1, which is a pharmaceutical composition that further comprises a pharmaceutically acceptable carrier.

C1. In certain non-limiting embodiments, the presently disclosed subject matter provides an immunoconjugate comprising the antibody or antigen-binding fragment thereof of any one of A1-A32, linked to a therapeutic agent.

C2. The foregoing immunoconjugate of C1, wherein the therapeutic agent is a drug, a cytotoxin, or a radioactive isotope.

D1. In certain non-limiting embodiments, the presently disclosed subject matter provides a composition comprising the immunoconjugate of C1 or C2.

D2. The foregoing composition of D1, which is a pharmaceutical composition that further comprises a pharmaceutically acceptable carrier.

E1. In certain non-limiting embodiments, the presently disclosed subject matter provides a multi-specific molecule comprising the antibody or antigen-binding fragment thereof of any one of A1-A32, linked to one or more functional moieties.

E2. The foregoing multi-specific molecule of E2, wherein the one or more functional moieties have a different binding specificity than the antibody or antigen binding fragment thereof.

F1. In certain non-limiting embodiments, the presently disclosed subject matter provides a composition comprising the multi-specific molecule of E1 or E2.

F2. The foregoing composition of F1, which is a pharmaceutical composition that further comprises a pharmaceutically acceptable carrier.

G1. In certain non-limiting embodiments, the presently disclosed subject matter provides a nucleic acid that encodes an antibody or antigen-binding fragment thereof of any one of A1-A32.

G2. In certain non-limiting embodiments, the presently disclosed subject matter provides a nucleic acid that encodes a heavy chain variable region of an antibody or antigen-binding fragment thereof of any one of A1-A32.

G3. The foregoing nucleic acid of G2, comprising a nucleotide sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the nucleotide sequence set forth in SEQ ID NO: 10, SEQ ID NO: 20, SEQ ID NO: 30, SEQ ID NO: 36, SEQ ID NO: 45, SEQ ID NO: 54, SEQ ID NO: 64, SEQ ID NO: 74, SEQ ID NO: 83, SEQ ID NO: 90, or SEQ ID NO: 93.

G4. The foregoing nucleic acid of G2 or G3, comprising the nucleotide sequence set forth in SEQ ID NO: 10, SEQ ID NO: 20, SEQ ID NO: 30, SEQ ID NO: 36, SEQ ID NO: 45, SEQ ID NO: 54, SEQ ID NO: 64, SEQ ID NO: 74, SEQ ID NO: 83, SEQ ID NO: 90, or SEQ ID NO: 93.

G5. In certain non-limiting embodiments, the presently disclosed subject matter provides a nucleic acid that encodes a light chain variable region of an antibody or antigen-binding fragment thereof of any one of A1-A32.

G6. The foregoing nucleic acid of G5, comprising a nucleotide sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the nucleotide sequence set forth in SEQ ID NO: 11, SEQ ID NO: 21, SEQ ID NO: 31, SEQ ID NO: 46, SEQ ID NO: 55, SEQ ID NO: 65, SEQ ID NO: 75, SEQ ID NO: 84, SEQ ID NO: 91, or SEQ ID NO: 94.

G7. The foregoing nucleic acid of G6 or G7, comprising the nucleotide sequence set forth in SEQ ID NO: 11, SEQ ID NO: 21, SEQ ID NO: 31, SEQ ID NO: 46, SEQ ID NO: 55, SEQ ID NO: 65, SEQ ID NO: 75, SEQ ID NO: 84, SEQ ID NO: 91, or SEQ ID NO: 94.

H1. In certain non-limiting embodiments, the presently disclosed subject matter provides a vector comprising the nucleic acid of any one of G1-G7.

J1. In certain non-limiting embodiments, the presently disclosed subject matter provides a host cell comprising the vector of H1.

K1. In certain non-limiting embodiments, the presently disclosed subject matter provides a method for detecting CD33 in a whole cell, a tissue, or a blood sample, comprising:
contacting a cell, tissue or blood sample with the antibody or antigen-binding fragment thereof of any one of A1-A32, wherein the antibody or antigen-binding fragment thereof comprises a detectable label; and
determining the amount of the labeled antibody or antigen-binding fragment thereof bound to the cell, tissue or blood sample by measuring the amount of detectable label associated with said cell or tissue, wherein the amount of bound antibody or antigen-binding fragment thereof indicates the amount of CD33 in the cell, tissue or blood sample.

L1. In certain non-limiting embodiments, the presently disclosed subject matter provides a method of treating or ameliorating a disease or disorder associated with CD33 in a subject, comprising administering to the subject the antibody or antigen-binding fragment thereof of any one of A1-A32, the immunoconjugate of C1 or C2, the multi-specific molecule of E1 or E2, or the composition of any one of B1, B2, D1, D2, F1, and F2.

L2. The foregoing method of L1, wherein the disease or disorder is a tumor.

L3. In certain non-limiting embodiments, the presently disclosed subject matter provides a method of reducing tumor burden in a subject, comprising administering to the subject an antibody or antigen-binding fragment thereof of any one of A1-A32, the immunoconjugate of C1 or C2, the multi-specific molecule of E1 or E2, or the composition of any one of B1, B2, D1, D2, F1, and F2.

L4. The foregoing method of L3, wherein the method reduces the number of the tumor cells, reduces the tumor size, and/or eradicates the tumor in the subject.

L5. In certain non-limiting embodiments, the presently disclosed subject matter provides a method of treating and/or preventing a tumor in a subject, comprising administering to the subject an antibody or antigen-binding fragment thereof of any one of A1-A32, the immunoconjugate of C1 or C2, the multi-specific molecule of E1 or E2, or the composition of any one of B1, B2, D1, D2, F1, and F2.

L6. In certain non-limiting embodiments, the presently disclosed subject matter provides a method of increasing or lengthening survival of a subject having a tumor, comprising administering to the subject an antibody or antigen-binding fragment thereof of any one of A1-A32, the immunoconjugate of C1 or C2, the multi-specific molecule of E1 or E2, or the composition of any one of B1, B2, D1, D2, F1, and F2.

L7. The foregoing method of L6, wherein the method reduces or eradicates tumor burden in the subject.

L8. The foregoing method of any one of L1-L7, wherein the tumor is cancer.

L9. The foregoing method of any one of L1-L8, wherein the tumor is hematological cancer or solid tissue cancer.

L10. The foregoing method of L9, wherein the hematological cancer is selected from the group consisting of acute myeloid leukemia (AML), myelodysplastic syndromes (MDS), acute lymphoblastic leukemia (ALL), chronic lymphocytic leukemia (CLL), myeloproliferative neoplasms (MPNs), and chronic myeloid neoplasms.

L11. The foregoing method of L10, wherein the hematological cancer is acute myeloid leukemia (AML).

L12. The foregoing method of any one of L1-L11, wherein the subject is a human.

M1. In certain non-limiting embodiments, the presently disclosed subject matter provides a kit for treating or ameliorating a disease or disorder in a subject, reducing tumor burden in a subject, treating and/or preventing a tumor in a subject, and/or increasing or lengthening survival of a subject having a tumor, comprising the antibody or antigen-binding fragment thereof of any one of A1-A32, the immunoconjugate of C1 or C2, the multi-specific molecule of E1 or E2, or the composition of any one of B1, B2, D1, D2, F1, and F2.

M2. The kit of claim 65, wherein the kit further comprises written instructions for using the antibody or antigen-binding fragment thereof, immunoconjugate, multi-specific molecule, or composition for treating or ameliorating a disease or disorder in a subject, treating or ameliorating a disease or disorder in a subject, reducing tumor burden in a subject, treating and/or preventing a tumor in a subject, and/or increasing or lengthening survival of a subject having a tumor.

### 6. EXAMPLES

The following examples are put forth so as to provide those of ordinary skill in the art with a complete disclosure and description of how to make and use the antibodies, multi-specific antibodies, compositions comprising thereof, screening, and therapeutic methods of the presently disclosed subject matter, and are not intended to limit the scope of what the inventors regard as their presently disclosed subject matter. It is understood that various other embodiments may be practiced, given the general description provided above.

### Example 1—Generation of the Presently Disclosed Anti-CD33 Antibodies and scFvs

Summary of Lead Identification. Antibody generation was carried out by immunizing AlivaMab® mice (Ablexis) with CD33 coding plasmid DNA and recombinant proteins either sourced commercially or produced in-house. Hybridoma were generated by electrofusion, and the supernatants were screened by enzyme-linked immunosorbent assay (ELISA) on recombinant proteins as well as by flow cytometry on cells overexpressing CD33 and AML cell lines with endogenous CD33 expression. After completion of subcloning and sequencing, 7 unique monoclonal antibodies (mAbs) were identified. Two antibodies bound to the IgC2 domain, and 5 to the IgV domain. Chimeric antigen receptors (CARs) were generated using variable Heavy (V_{H}) and variable Light (V_{L}) chain sequences formatted into single chain fragment variable (scFvs) domains as building blocks.

The 3-P14 and 4-B2 antibodies were selected as the front-runner lead candidates based on the following biological and functional properties:
Membrane-proximal epitope (IgC2 domain)
Strong binding to various AML lines with single- to double-digit nM EC50

One additional antibody, 1J19 which targets membrane-distal IgV domain, also exhibited superior potency when compared to the reference antibody. As a result, this clone was included as a backup molecule.

Antibody Generation. Recombinant human CD33 protein was sourced commercially from R&D Systems and Acro Biosystems. Mouse and cynomolgus monkey CD33 proteins were purchased from Sino Biological. The extracellular domains (ECD) of human CD33 (residues 18-259; European Nucleotide Archive #AAA51948) fused via carboxy-terminus to human IgG1 Fc or a 6xHis-tag, as well as cynomolgus monkey CD33 (residues 35-275) and mouse CD33 (residues 18-240) fused via their carboxy-terminus to a 6xHis-tag, were used. In addition, the human CD33 IgC2 type domain (residue 140-259) with a terminal mouse IgG1 Fc or 6xHis-tag, was produced at LakePharma.

DNA coding for human CD33 IgC2 extracellular domain or IgC2 followed by the CD33 transmembrane segment was cloned into vector for DNA immunizations. All proteins and DNA reagents were subjected to rigorous quality control prior to use for antibody generation, screening and characterization. Multiple cohorts of AlivaMab® mice were immunized with recombinant proteins or plasmid DNA coding for the CD33 IgC2 type domain as shown in Table 13. The immunization approach for cohorts 2 and 3 aimed at developing cross-reactive antibodies, whereas the strategy for cohorts 4-6 was designed to generate membrane-proximal domain antibodies. Complete Freund's Adjuvant (CFA) was used for the prime injection, followed by 8-12 boosts in TiterMax or RIBI over the course of 28-34 days. Proteins were administered via the hock route, while DNA was injected into the tail vein.

TABLE 13
Immunization cohorts and immunogens¹

| Cohort | Number of | Immunogen |
|---|---|---|
| 1 | 4 × AMM K/X | huCD33-ECD Fc chimera |
| 2 | 4 × AMM K/X | huCD33-ECD Fc chimera + final boost |
| | | cynoCD33- |
| 3 | 4 × AMM K/X | huCD33-ECD Fc chimera + moCD33-ECD His |
| 4 | 4 × AMM K/X | huCD33-ECD-IgC2 moFc chimera |
| 5 | 3 × AMM K/X | DNA coding for huCD33-IgC2-TM |
| 6 | 3 × AMM K/X | DNA coding for huCD33-ECD-IgC2-His |

¹AMM K/X: AlivaMab ® mice with kappa and lambda human light chains: hu: human; mo: mouse; cyno: cynomolgus.

Serum was collected on days 17, 24, 28 and 31, and the immune response was analyzed by ELISA using recombinant human CD33 ECD-6xHis and CD33 IgC2-6xHis proteins. Mice from cohorts 1, 2 and 4 developed midpoint titers greater than 1:10,000 and were selected for the final boost, hybridoma generation and screening.

Three days after the final boost, lymph nodes were collected and pooled for each cohort, followed by isolation of IgG-producing B cells by magnetic sorting. The enriched B cells were electrofused with mouse myeloma cells to generate hybridoma, which were plated for screening.

Hybridoma screening. Hybridoma supernatants were analyzed by screening. Binding to human CD33 (full ECD and IgC2), cynomolgus monkey and mouse CD33 was analyzed by ELISA. Tertiary screening was carried out by flow cytometry utilizing cells stably overexpressing human CD33 (NIH-3T3 full-length and IgC2 domain only) and AML cell lines that endogenously express CD33 (U937). NALM6 cells, which are CD33 negative, were used to test for non-specific binding.

The primary screen by ELISA using soluble, His-tagged human CD33 (full-length and IgC2) resulted in 96 hits. A secondary screen using human, cynomolgus and mouse CD33 resulted in 53 confirmed binders. In addition, 18 clones were cross-reactive with cynomolgus monkey CD33, and 3 supernatants exhibited weak binding to mouse CD33. All 53 confirmed supernatants were tested for binding to CD33 in its native, membrane-bound form on 3T3 cells overexpressing human CD33 (full-length or IgC2 domain). Furthermore, 31 supernatants gave a signal above background on these cells, with 5 clones targeting the IgC2 domain. Binding was tested on the AML cell lines U937 and Set2. All 31 clones were confirmed on these AML lines.

Upon completion of the binding and cross-reactivity experiments delineated in the screening funnel, 10 hybridoma were selected for subcloning. These included 4 antibodies that were cross-reactive with cynomolgus CD33 and 3 antibodies were IgC2 domain specific. All 10 clones showed consistent binding when tested by ELISA (see Table 14), or flow cytometry on over expressing as well as AML lines (see Table 15).

TABLE 14
Binding of selected parental clones to recombinant proteins².

| | huCD33- | cynoCD33- | moCD33- | huCD33IgC2- |
|---|---|---|---|---|
| Antibody | His | His | His | His |
| 1A20 | 117477 | 35495 | 238 | 257 |
| 1H19 | 131738 | 22511 | 653 | 58888 |
| 1J19 | 95614 | 2343 | 1804 | 15162 |
| 1P13 | 116818 | 100568 | 1806 | 2553 |
| 1P23 | 41802 | 5160 | 259 | 147 |
| 2F18 | 251476 | 197236 | 4007 | 20388 |
| 2N3 | 723580 | 3911 | 1730 | 1794 |
| 3P14 | 545702 | 12656 | 13674 | 107398 |
| 4B2 | 392249 | 589 | 383 | 101041 |
| 4P3 | 484133 | 548 | 429 | 429 |

²Relative light unit (RLU) values given. A signal greater than 10,000 was considered specific.

TABLE 15
Binding of selected parental hybridoma supernatants
to CD33⁺ and CD33⁻ cells³.

| | 3T3- | 3T3- | | | Parental- |
|---|---|---|---|---|---|
| Antibody | huCD33 | huCD33IgC2 | U937 | NALM6 | 3T3 |
| 1A20 | 1978083 | 7950 | 558301 | 5271 | 4446 |
| 1H19 | 1752820 | 10513 | 479140 | 5694 | 4334 |
| 1J19 | 3858744 | 8034 | 338841 | 6469 | 5074 |
| 1P13 | 2152724 | 23880 | 558987 | 9734 | 6777 |
| 1P23 | 4082519 | 7728.5 | 455774 | 6069 | 4447 |
| 2F18 | 3563970 | 8064 | 351310 | 6326 | 4579 |
| 2N3 | 4211927 | 7896 | 409016 | 6862 | 4849 |
| 3P14 | 3059525 | 543669 | 248143 | 6656 | 5180 |
| 4B2 | 2820034 | 357782 | 152109 | 5389 | 4746 |
| 4P3 | 1319440 | 391433 | 187730 | 5708 | 4849 |

³Mean fluorescence intensity (MFI) values given. A signal >100,000 was used as cut off.

Subcloning. Subcloning was carried out by limiting dilution. Four subclones of each parent hybridoma were selected and tested by flow cytometry using 3T3 CD33, 3T3 CD33-IgC2, 3T3 parental, U937 and NALM6 cell lines.

### Example 2—Binding Characterization of the Presently Disclosed Anti-CD33 Antibodies

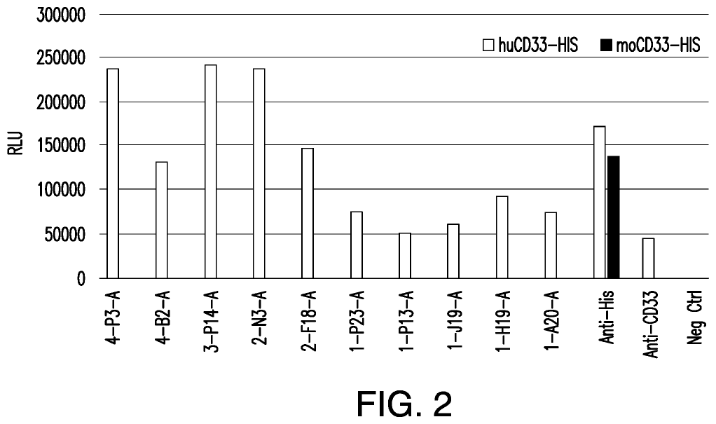

One subclone from each parental antibody was selected and purified from a 30-ml hybridoma supernatant culture using Protein G or Protein A depending on the murine IgG isotype subclass. The purified antibodies were characterized by FACS and ELISA as follows:
ELISA binding to huCD33(ECD)-His and moCD33(ECD)-His;
FACS binding to huCD33 (FL)-3T3, U937 and U937 hCD33KO cells; and
FACS EC50 on two different CD33 positive AML lines (U937 and Set2; see FIG. 2 and Table 16)

TABLE 16
Binding of 10 clonal mAbs to two AML lines

| Antibody | U937 EC50 (nM) | Set2 EC50 (nM) |
|---|---|---|
| 1-A20-A | 0.6 | 0.4 |
| 1-H19-A | 0.866 | 0.4 |
| 1-J19-A | 10.2 | 7.93 |
| 1-P13-A | 6.33 | 5 |
| 1-P23-A | 1.07 | 1.4 |
| 2-F18-A | 11.87 | 24.2 |
| 2-N3-A | 0.4 | 0.466 |
| 3-P14-A | 3.27 | 2.4 |
| 4-B2-A | 40.53 | 28.53 |
| 4-P3-A | 2.5 | 2 |

In order to establish the target candidate profile (TCP), 3P14 and 4B2 in V_{H}-V_{L} scFv orientation (designated as “TDI-Y-006” and “TDI-Y-007”, respectively) and 1J19 were selected. The targets were tested in a series of in vitro binding and functional assays. For binding studies, the antibodies were produced recombinantly with a human IgG1 constant region. Antibody H195 (lintuzumab) was included as a reference for comparison.

TDI-Y-006 and TDI-Y-007 bound to recombinant human CD33 in solution with single-digit nM affinity (K_{D}=1.2 and 1.29 nM, respectively). Cross-reactivity with cynomolgus monkey and mouse CD33 was not observed. TDI-Y-006 and TDI-Y-007 bound to a NIH-3T3 cell line overexpressing CD33 with EC50 of 6.54 nM and 17 nM, respectively. In addition, TDI-Y-006 and TDI-Y-007 bound to U937 cells, a human AML cell line that endogenously expresses CD33 with EC50 values of 8.5 nM and 45 nM, respectively.

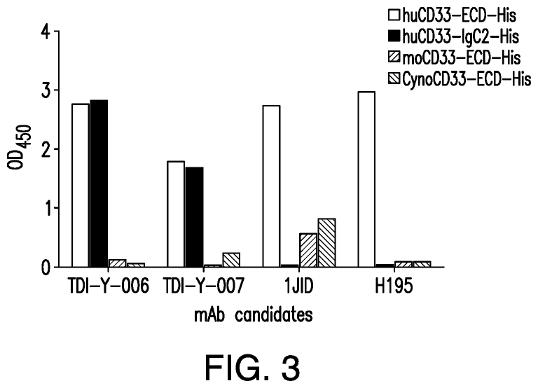

Binding to Recombinant CD33 Protein. Binding to CD33 protein was carried out by ELISA using human CD33-His (full-length or IgC2 domain), cynomolgus monkey CD33-His and mouse CD33-His. Recombinant proteins at 5 μg/mL were captured by pre-blocked Ni-NTA plates. Candidate antibodies were added at 10 μg/mL in triplicate and detected using horseradish peroxidase (HRP)-conjugated anti-human Fc antibody. As shown in FIG. 3, TDI-Y-006 and TDI-Y-007 bound to both human CD33 ECD and IgC2 domain protein. No binding was detected to cynomolgus or mouse CD33 for either TDI-Y-006 and TDI-Y-007. 1J19 and H195 only bound to full-length human CD33 ECD but not to IgC2 as these antibodies target the IgV domain. 1J19 showed moderate levels of cross-reactivity to cynoCD33.

The binding affinity to human CD33 protein was measured by biolayer interferometry (BLI) using an Octet Red96e. All experiments were carried out using kinetic buffer (PBS pH 7.4, 0.01% BSA, 0.002% Tween-20). The antibodies were captured by an anti-huFc biosensor and a 7-point, 2-fold dilution series of huCD33-His was used as analyte. The data was processed by double reference subtraction, and response curves were globally fit to a 1:1 Langmuir binding model. The results are shown in Table 17 below. TDI-Y-006, TDI-Y-007, and 1J19 had dissociation constants (K_{D}) of 1-2 nM, with slight variations in on-rate and off-rate. The binding affinities are similar to the H195 reference antibody.

TABLE 17
Binding kinetics of TDI-Y-006, TDI-
Y-007 and 1J19 to CD33 in solution.

| Antibody | kon (1/Ms) | koff | K_{D} (nM) |
|---|---|---|---|
| TDI-Y-006 | 2.96 × 10⁵ | 3.59 × 10⁻⁴ | 1.21 |
| TDI-Y-007 | 4.64 × 10⁵ | 5.97 × 10⁻⁵ | 1.29 |
| 1J19 | 2.66 × 10⁵ | 2.93 × 10⁻⁴ | 1.1 |
| H195 | 3.58 × 10⁵ | 4.38 × 10⁻⁴ | 1.22 |

The binding affinity to cynomolgus monkey CD33 was also measured by BLI using an Octet Red96e as described above. None of the antibodies, including H195, showed binding to cynomolgus monkey CD33 at 500 nM concentration.

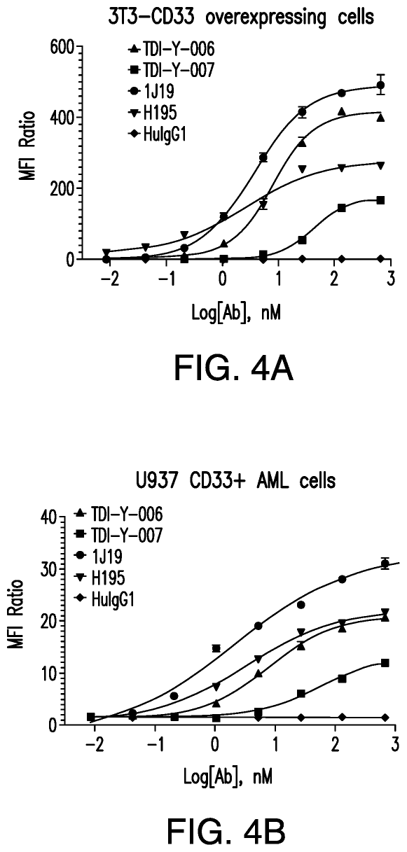

Binding to CD33 on Cells. To assess the binding of TDI-Y-006, TDI-Y-007 and 1J19 to cell surface-bound CD33, 3T3 cells overexpressing CD33, as well as the U937 AML cell line, which expresses endogenous CD33, were used. U937 CD33KO or NALM6 were included as negative controls. Briefly, NALM6, U937 CD33+ and KO cells were blocked with human IgG Fc for 20 minutes on ice. The recombinant antibodies were then serially diluted starting at 100 μg/mL concentration and added for 30 minutes on ice. AlexaFluor 647-conjugated goat anti-human F(ab′)2 was added to cells for 30 minutes on ice, washed and analyzed by flow cytometry, normalized to secondary only staining (MFI ratio). EC50 values were determined by non-linear regression. TDI-Y-006 bound to 3T3-CD33 overexpressing and U937 cells with EC50 values of 5 and 8.5 nM, respectively. TDI-Y-007 bound 3T3-CD33 and U937 cells with EC50 values of 17 and 45 nM, respectively. 1J19 and H195 bound 3T3-CD33 and U937 cells with EC50 values in the single-digit nM range (FIGS. 4A and 4B; and Table 18). No binding was observed with NALM6 (CD33 negative) or U937CD33KO at 666 nM (data not shown).

TABLE 18
EC50 of tested antibodies on overexpressing
(3T3) and AML line (U937)

| Antibody | 3T3-CD33 EC50 (nM) | U937 EC50 (nM) |
|---|---|---|
| TDI-Y-006 | 5 | 8.5 |
| TDI-Y-007 | 17 | 44.9 |
| 1J19 | 6.54 | 2.16 |
| H195 | 4.8 | 0.4 |

A 40-fold difference between K_{D} in solution and EC50 on cells was observed for TDI-Y-007. The epitope for this antibody may be close to the cell membrane and thus less accessible on cells than to a soluble protein. The epitope of TDI-Y-006, which has K_{D} and EC50 values within 5- to 7-fold and was distinct from TDI-Y-007 (see below), may be located in a part of the IgC2 domain that is equally accessible in both recombinant protein as well as on cell surface.

Epitope Binning. Epitope binning was carried out by a BLI competition experiment using an Octet Red96e. Human CD33-His was captured by an anti-penta-His biosensor. A 4×4 matrix with TDI-Y-006, TDI-Y-007, 1J19 and H195 was tested. A reference biosensor was used to determine the overall capture level for each antibody. After pre-binding the antibodies at saturation levels, the biosensors were dipped into antibody solutions to assess competition. The data is shown in Table 19. When TDI-Y-007 was pre-bound, TDI-Y-006 still showed significant capture. However, when TDI-Y-006 was pre-bound, binding of TDI-Y-007 was blocked. This suggests that TDI-Y-006 and TDI-Y-007 have epitopes that are in close proximity or may be partially overlapping but are not identical. Similarly, 1J19 and H195 have epitopes that partially overlap. No competition was observed between antibodies binding to the membrane-proximal IgC2 domain (TDI-Y-006 and TDI-Y-007) and antibodies binding to the membrane-distal IgV domain (1J19 and H195).

TABLE 19
Epitope Binning of CD33 lead mAbs⁴.

| Antibody | TDI-Y-006 | TDI-Y-007 | 1J19 | H195 |
|---|---|---|---|---|
| TDI-Y-006 | 0.1185 | 0.0001 | 0.3922 | 0.5819 |
| TDI-Y-007 | 0.5826 | 0.1576 | 0.4375 | 0.743 |
| 1J19 | 0.8614 | 0.3356 | 0.0683 | 0.4154 |
| H195 | 0.7363 | 0.2284 | 0.028 | 0.0356 |
| Reference Biosensor | 0.8595 | 0.2991 | 0.432 | 0.743 |

⁴The values under “Reference Biosensor” indicate the total binding capacity of each antibody to CD33. The lower the value compared to reference sensor, the higher the degree of competition between two pairs of antibodies tested.

A summary of the characteristics of the TDI-Y-006 and TDI-Y-007 antibodies is provided in Table 20 below.

TABLE 20
Pharmacological Properties

| Parameter | TDI-Y-006 | TDI-Y-007 |
|---|---|---|
| Binding affinity to | K_{D}: 1.2 nM | K_{D}: 1.29 nM |
| soluble CD33 | kₒₙ: 2.96 × 10⁵ M⁻¹s⁻¹ | kₒₙ: 4.64 × 105 M⁻¹s⁻¹ |
| | k_{off}: 3.59 × 10⁻⁴ s⁻¹ | K_{off}: 5.97 × 10⁻⁵ s⁻¹ |
| Binding to 3T3 cells | EC50 = 5 nM | EC50 = 17 nM |
| overexpressing CD33 | | |
| Binding to AML cell | EC50 = 8.5 nM | EC50 = 45 nM |
| line (U937) | | |
| Cross-reactivity to | No binding at 500 nM | No binding at 500 nM |
| cynomolgus CD33 | | |
| Cross-reactivity to | No binding at 500 nM | No binding at 500 nM |
| mouse CD33 | | |

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An anti-CD33 antibody or an antigen-binding fragment thereof, comprising:
(a) a heavy chain variable region comprising a CDR1 comprising the amino acid sequence set forth in SEQ ID NO: 2, a CDR2 comprising the amino acid sequence set forth in SEQ ID NO: 3, and a CDR3 comprising the amino acid sequence set forth in SEQ ID NO: 4; and a light chain variable region comprising a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7;
(b) a heavy chain variable region comprising a CDR1 comprising the amino acid sequence set forth in SEQ ID NO: 12, a CDR2 comprising an amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17;
(c) a heavy chain variable region comprising a CDR1 comprising the amino acid sequence set forth in SEQ ID NO: 22, a CDR2 comprising the amino acid sequence set forth in SEQ ID NO: 23, and a CDR3 comprising the amino acid sequence set forth in SEQ ID NO: 24; and a light chain variable region comprising a CDR1 comprising the amino acid sequence set forth in SEQ ID NO: 25, a CDR2 comprising the amino acid sequence set forth in SEQ ID NO: 26, and a CDR3 comprising the amino acid sequence set forth in SEQ ID NO: 27;
(d) a heavy chain variable region comprising a CDR1 comprising the amino acid sequence set forth in SEQ ID NO: 32, a CDR2 comprising the amino acid sequence set forth in SEQ ID NO: 33, and a CDR3 comprising the amino acid sequence set forth in SEQ ID NO: 34; and a light chain variable region comprising a CDR1 comprising the amino acid sequence set forth in SEQ ID NO: 25, a CDR2 comprising the amino acid sequence set forth in SEQ ID NO: 26, and a CDR3 comprising the amino acid sequence set forth in SEQ ID NO: 27;
(c) a heavy chain variable region comprising a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39; and a light chain variable region comprising a CDR1 comprising the amino acid sequence set forth in SEQ ID NO: 40, a CDR2 comprising the amino acid sequence set forth in SEQ ID NO: 41, and a CDR3 comprising the amino acid sequence set forth in SEQ ID NO: 42;
(f) a heavy chain variable region comprising a CDR1 comprising the amino acid sequence set forth in SEQ ID NO: 47, a CDR2 comprising the amino acid sequence set forth in SEQ ID NO: 38, and a CDR3 comprising the amino acid sequence set forth in SEQ ID NO: 48; and a light chain variable region comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51;
(g) a heavy chain variable region comprising a CDR1 comprising the amino acid sequence set forth in SEQ ID NO: 56, a CDR2 comprising the amino acid sequence set forth in SEQ ID NO: 57, and a CDR3 comprising the amino acid sequence set forth in SEQ ID NO: 58; and a light chain variable region comprising a CDR1 comprising the amino acid sequence set forth in SEQ ID NO: 59, a CDR2 comprising the amino acid sequence set forth in SEQ ID NO: 60, and a CDR3 comprising the amino acid sequence set forth in SEQ ID NO: 61;
(h) a heavy chain variable region comprising a CDR1 comprising the amino acid sequence set forth in SEQ ID NO: 66, a CDR2 comprising the amino acid sequence set forth in SEQ ID NO: 67, and a CDR3 comprising the amino acid sequence set forth in SEQ ID NO: 68; and a light chain variable region comprising a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71;
(i) a heavy chain variable region comprising a CDR1 comprising the amino acid sequence set forth in SEQ ID NO: 76, a CDR2 comprising the amino acid sequence set forth in SEQ ID NO: 57, and a CDR3 comprising the amino acid sequence set forth in SEQ ID NO: 77; and a light chain variable region comprising a CDR1 comprising the amino acid sequence set forth in SEQ ID NO: 78, a CDR2 comprising the amino acid sequence set forth in SEQ ID NO: 79, and a CDR3 comprising the amino acid sequence set forth in SEQ ID NO: 80; or
(j) a heavy chain variable region comprising a CDR1 comprising the amino acid sequence set forth in SEQ ID NO: 85, a CDR2 comprising the amino acid sequence set forth in SEQ ID NO: 86, and a CDR3 comprising the amino acid sequence set forth in SEQ ID NO: 87; and a light chain variable region comprising a CDR1 comprising the amino acid sequence set forth in SEQ ID NO: 5, a CDR2 comprising the amino acid sequence set forth in SEQ ID NO: 41, and a CDR3 comprising the amino acid sequence set forth in SEQ ID NO: 42.

2. The antibody or antigen-binding fragment thereof of claim 1, wherein:
(a) a heavy chain variable region comprising a CDR1 comprising the amino acid sequence set forth in SEQ ID NO: 2, a CDR2 comprising the amino acid sequence set forth in SEQ ID NO: 3, and a CDR3 comprising the amino acid sequence set forth in SEQ ID NO: 4; and a light chain variable region comprising a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7;
(b) a heavy chain variable region comprising a CDR1 comprising the amino acid sequence set forth in SEQ ID NO: 12, a CDR2 comprising an amino acid sequence set forth in SEQ ID NO: 13, and a CDR3 comprising the amino acid sequence set forth in SEQ ID NO: 14; and a light chain variable region comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17; or
(c) a heavy chain variable region comprising a CDR1 comprising the amino acid sequence set forth in SEQ ID NO: 22, a CDR2 comprising the amino acid sequence set forth in SEQ ID NO: 23, and a CDR3 comprising the amino acid sequence set forth in SEQ ID NO: 24; and a light chain variable region comprising a CDR1 comprising the amino acid sequence set forth in SEQ ID NO: 25, a CDR2 comprising the amino acid sequence set forth in SEQ ID NO: 26, and a CDR3 comprising the amino acid sequence set forth in SEQ ID NO: 27.

3. The antibody or antigen-binding fragment thereof of claim 1, comprising
(a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, SEQ ID NO: 18, SEQ ID NO: 28, SEQ ID NO: 35, SEQ ID NO: 43, SEQ ID NO: 52, SEQ ID NO: 62, SEQ ID NO: 72, SEQ ID NO: 81, or SEQ ID NO: 88; and
(b) a light chain variable region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 9, SEQ ID NO: 19, SEQ ID NO: 29, SEQ ID NO: 44, SEQ ID NO: 53, SEQ ID NO: 63, SEQ ID NO: 73, SEQ ID NO: 82, SEQ ID NO: 89, or SEQ ID NO: 92.

4. The antibody or antigen-binding fragment thereof of claim 1, comprising:
(a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9;
(b) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 18, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 19;
(c) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 28, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 29;
(d) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 35, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 29;
(e) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 43, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 44;
(f) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 52, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 53;
(g) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 62, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 63;
(h) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 72, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 73;
(i) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 81, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 81;
(j) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 88, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 89; or
(k) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 92.

5. The antibody or antigen-binding fragment thereof claim 4, wherein
(a) the heavy chain variable region comprises the amino acid sequence set forth in SEQ ID NO: 8, and the light chain variable region comprises the amino acid sequence set forth in SEQ ID NO: 9;
(b) the heavy chain variable region comprises the amino acid sequence set forth in SEQ ID NO: 18, and the light chain variable region comprises the amino acid sequence set forth in SEQ ID NO: 19;
(c) the heavy chain variable region comprises the amino acid sequence set forth in SEQ ID NO: 28, and the light chain variable region comprises the amino acid sequence set forth in SEQ ID NO: 29;
(d) the heavy chain variable region comprises the amino acid sequence set forth in SEQ ID NO: 35, and the light chain variable region comprises the amino acid sequence set forth in SEQ ID NO: 29;
(e) the heavy chain variable region comprises the amino acid sequence set forth in SEQ ID NO: 43, and the light chain variable region comprises the amino acid sequence set forth in SEQ ID NO: 44;
(f) the heavy chain variable region comprises the amino acid sequence set forth in SEQ ID NO: 52, and the light chain variable region comprises the amino acid sequence set forth in SEQ ID NO: 53;
(g) the heavy chain variable region comprises the amino acid sequence set forth in SEQ ID NO: 62, and the light chain variable region comprises the amino acid sequence set forth in SEQ ID NO: 63;
(h) the heavy chain variable region comprises the amino acid sequence set forth in SEQ ID NO: 72, and the light chain variable region comprises the amino acid sequence set forth in SEQ ID NO: 73;
(i) the heavy chain variable region comprises the amino acid sequence set forth in SEQ ID NO: 81, and the light chain variable region comprises the amino acid sequence set forth in SEQ ID NO: 82;
(j) the heavy chain variable region comprises the amino acid sequence set forth in SEQ ID NO: 88, and the light chain variable region comprises the amino acid sequence set forth in SEQ ID NO: 89; or
(k) the heavy chain variable region comprises the amino acid sequence set forth in SEQ ID NO: 98, and the light chain variable region comprises the amino acid sequence set forth in SEQ ID NO: 92.

6. The anti-CD33 antibody or an antigen-binding fragment thereof of claim 5, wherein:
(a) the heavy chain variable region comprises the amino acid sequence set forth in SEQ ID NO: 8, and the light chain variable region comprises the amino acid sequence set forth in SEQ ID NO: 9;
(b) the heavy chain variable region comprises the amino acid sequence set forth in SEQ ID NO: 18, and the light chain variable region comprises the amino acid sequence set forth in SEQ ID NO: 19; or
(c) the heavy chain variable region comprises the amino acid sequence set forth in SEQ ID NO: 28, and the light chain variable region comprises the amino acid sequence set forth in SEQ ID NO: 29.

7. The antibody or antigen-binding fragment thereof of claim 1, wherein the antibody comprises a comprises a heavy chain constant region and/or a light chain constant region.

8. The antibody or antigen-binding fragment thereof of claim 7, wherein:
(a) the heavy chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 95; and/or
(b) the light chain constant region comprises an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 96.

9. The antibody or antigen-binding fragment thereof of claim 7, wherein:
(a) the heavy chain constant region comprises the amino acid sequence set forth in SEQ ID NO: 95; and/or
(b) the light chain constant region comprises the amino acid sequence set forth in SEQ ID NO: 96 or SEQ ID NO: 99.

10. The antibody or antigen-binding fragment thereof of claim 1, wherein the antibody or an antigen-binding fragment thereof comprises:
(a) a heavy chain comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 97, SEQ ID NO: 100, or SEQ ID NO: 102; and/or
(b) a light chain comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 98, SEQ ID NO: 101, or SEQ ID NO: 103.

11. The antibody or antigen-binding fragment thereof of claim 1, wherein the antibody or an antigen-binding fragment thereof comprises:
(a) a heavy chain comprising the amino acid sequence set forth in SEQ ID NO: 97, SEQ ID NO: 100, or SEQ ID NO: 102; and/or
(b) a light chain comprising the amino acid sequence set forth in SEQ ID NO: 98, SEQ ID NO: 101, or SEQ ID NO: 103.

12. The antibody or antigen-binding fragment thereof of claim 1, wherein (a) the antibody comprises a human variable region framework region; (b) the antibody is a fully human antibody or an antigen-binding fragment thereof; (c) the antibody is a chimeric antibody or an antigen-binding fragment thereof; or (d) the antibody is a humanized antibody or an antigen-binding fragment thereof.

13. The antibody or antigen-binding fragment thereof of claim 1, wherein the antigen-binding fragment is a Fab, Fab′, F(ab′)₂, variable fragment (Fv), or single chain variable region (scFv).

14. A composition comprising the antibody or antigen-binding fragment thereof of claim 1.

15. An immunoconjugate comprising the antibody or antigen-binding fragment thereof of claim 1, linked to a therapeutic agent.

16. The immunoconjugate of claim 15, wherein the therapeutic agent is a drug, a cytotoxin, or a radioactive isotope.

17. A composition comprising the immunoconjugate of claim 14.

18. A multi-specific molecule comprising the antibody or antigen-binding fragment thereof of claim 1, linked to one or more functional moieties.

19. A composition comprising the multi-specific molecule of claim 18.

20. A nucleic acid that encodes an antibody or antigen-binding fragment thereof of claim 1.

21. A vector comprising the nucleic acid of claim 20.

22. A host cell comprising the vector of claim 21.

23. A method for detecting CD33 in a whole cell, a tissue, or a blood sample, comprising:
contacting a cell, tissue or blood sample with the antibody or antigen-binding fragment thereof of claim 1, wherein the antibody or antigen-binding fragment thereof comprises a detectable label; and
determining the amount of the labeled antibody or antigen-binding fragment thereof bound to the cell, tissue or blood sample by measuring the amount of detectable label associated with said cell or tissue, wherein the amount of bound antibody or antigen-binding fragment thereof indicates the amount of CD33 in the cell, tissue or blood sample.

24. A method of treating or ameliorating a disease or disorder associated with CD33, reducing tumor burden in a subject, treating and/or preventing a tumor in a subject, and/or increasing or lengthening survival of a subject, comprising administering to the subject an antibody or antigen-binding fragment thereof of claim 1, wherein the tumor is associated with CD33.

25. The method of claim 24, wherein the tumor is hematological cancer or solid tissue cancer.

26. The method of claim 25, wherein the hematological cancer is selected from the group consisting of acute myeloid leukemia (AML), myelodysplastic syndromes (MDS), acute lymphoblastic leukemia (ALL), chronic lymphocytic leukemia (CLL), myeloproliferative neoplasms (MPNs), and chronic myeloid neoplasms.

27. A kit for treating or ameliorating a disease or disorder in a subject, reducing tumor burden in a subject, treating and/or preventing a tumor in a subject, and/or increasing or lengthening survival of a subject having a tumor, comprising the antibody or antigen-binding fragment thereof claim 1.


SEQUENCE LISTING (109 sequences)

SEQ ID NO: 1 - protein, 364 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 2 - protein, 8 aa, synthetic construct
GFTFSTYA

SEQ ID NO: 3 - protein, 8 aa, synthetic construct
ISGRGGST

SEQ ID NO: 4 - protein, 14 aa, synthetic construct
AGRGDYYYYYGMDV

SEQ ID NO: 5 - protein, 11 aa, synthetic construct
QSLVYSDGNTY

SEQ ID NO: 6 - skipped

SEQ ID NO: 7 - protein, 9 aa, synthetic construct
MQSTQFPHT

SEQ ID NO: 8 - protein, 121 aa, synthetic construct
EVQLLESGGGLVQPGGSLRLSCAASGFTFSTYAMSWVRQAPGKGLEWVSAISGRGGSTYYTDSVKGRFTISRDNSKNTVSLQMNSLRAEDTAVYYCAGRGDYYYYYGMDVWGQGTTVTVSA

SEQ ID NO: 9 - protein, 112 aa, synthetic construct
DIVMTQSPLSSPVTLGQPASFSCRSSQSLVYSDGNTYLSWLQQRPGQPPRLLIYKISNRFSGVPDRFSGSGAGTDFTLKISRVEAEDVGVYYCMQSTQFPHTFGQGTKLEIK

SEQ ID NO: 10 - DNA, 363 bp, synthetic construct
  source 1..363: mol_type = other DNA
gaggtgcagctgttggagtctgggggaggcttggtccagcctggggggtccctgagactctcctgtgcagcctctggattcacctttagcacctatgccatgagctgggtccgccaggctccagggaaggggctggagtgggtctcagctattagtggtcgtggtggtagcacatactacacagactccgtgaagggccggttcaccatctccagagacaattccaagaacacggtgtctctgcaaatgaacagcctgagagccgaggacacggccgtatattactgtgcgggccggggagattactactactactacggtatggacgtctggggccaagggaccacggtcaccgtctccgca

SEQ ID NO: 11 - DNA, 336 bp, synthetic construct
  source 1..336: mol_type = other DNA
gatattgtgatgacccagagtccactctcctcacctgtcacccttggacagccggcctccttctcctgcaggtctagtcaaagcctcgtatacagtgatggaaacacctacttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcatgcaatctacacaatttcctcacacttttggccaggggaccaagctggagatcaaa

SEQ ID NO: 12 - protein, 8 aa, synthetic construct
GFIFSSNA

SEQ ID NO: 13 - protein, 8 aa, synthetic construct
ISGYGGNT

SEQ ID NO: 14 - protein, 14 aa, synthetic construct
AKWGTYIVGATGDY

SEQ ID NO: 15 - protein, 9 aa, synthetic construct
SNDVGGYNY

SEQ ID NO: 16 - skipped

SEQ ID NO: 17 - protein, 10 aa, synthetic construct
SSYAGSNNWV

SEQ ID NO: 18 - protein, 121 aa, synthetic construct
EVHLLESGGGLVQPGGSLRLSCAASGFIFSSNAMSWVRQAPGKGLEWVSAISGYGGNTYYADSVKGRFTISRDNSKNTLYLQMNSLRAEDTAVYYCAKWGTYIVGATGDYWGQGTLVTVSS

SEQ ID NO: 19 - protein, 110 aa, synthetic construct
QSALTQPPSASGSPGQSVTISCTGTSNDVGGYNYVSWYQQHPGKAPKLLIYEVSKRPSGVPDRFSGSQSGNTASLTVSGLQAEDEADYYCSSYAGSNNWVFGGGTKLTVL

SEQ ID NO: 20 - DNA, 363 bp, synthetic construct
  source 1..363: mol_type = other DNA
gaggtgcacctgttggagtctgggggaggcttggtacagcctggggggtccctgagactctcctgtgcagcctctggattcatctttagcagcaatgccatgagctgggtccgccaggctccagggaagggactggagtgggtctcagctattagtggttatggtggtaacacatactacgcagactccgtgaagggccggttcaccatctccagagacaattccaagaacacgctatatctgcaaatgaacagcctgagagccgaggacacggccgtatattactgtgcgaaatgggggacttatatagtgggagctacgggtgactactggggccagggaactctggtcaccgtctcctca

SEQ ID NO: 21 - DNA, 330 bp, synthetic construct
  source 1..330: mol_type = other DNA
cagtctgccctgactcagcctccctccgcgtccgggtctcctggacagtcagtcaccatctcctgcactggaaccagcaatgacgttggtggttataactatgtctcctggtaccaacagcacccaggcaaagcccccaaactcttgatttatgaggtcagtaagcggccctcaggggtccctgatcgcttctctggctcccagtctggcaacacggcctccctgaccgtctctgggctccaggctgaggatgaggctgattattactgcagctcatatgcaggcagcaacaattgggtgttcggcggagggaccaagctgaccgtccta

SEQ ID NO: 22 - protein, 10 aa, synthetic construct
GDSVSSNSAA

SEQ ID NO: 23 - protein, 9 aa, synthetic construct
TYFRSKWYN

SEQ ID NO: 24 - protein, 10 aa, synthetic construct
ASEGGSYYDH

SEQ ID NO: 25 - protein, 6 aa, synthetic construct
QGISNW

SEQ ID NO: 26 - skipped

SEQ ID NO: 27 - protein, 9 aa, synthetic construct
QQADSFPFT

SEQ ID NO: 28 - protein, 120 aa, synthetic construct
QVQLQQSGPGLVKPSQTLSLTCAISGDSVSSNSAAWNWIRQSPSRGLEWLGRTYFRSKWYNVYAVSVKSRITINPDTSKNQFSLQLNSVTPEDTAVYYCASEGGSYYDHWGQGTLVTVSS

SEQ ID NO: 29 - protein, 107 aa, synthetic construct
DIQMTQSPSSVSASVGDRVTITCRASQGISNWLTWYQQKPGKAPKLLIYAASSLQSGVPSRFSGSGSGTDFTLTISSLQPEDFATYYCQQADSFPFTFGPGTKVDIK

SEQ ID NO: 30 - DNA, 360 bp, synthetic construct
  source 1..360: mol_type = other DNA
caggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatacttcaggtccaagtggtataatgtttatgcagtgtctgtgaagagtcgaataaccatcaacccagacacatccaagaaccagttctccctgcagctgaactctgtgactcccgaggacacggctgtgtattattgtgcaagcgagggtgggagctattatgaccactggggccagggaaccctggtcaccgtctcctca

SEQ ID NO: 31 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagtaattggttaacctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctgacagtttcccattcactttcggccctgggaccaaagtggatatcaaa

SEQ ID NO: 32 - protein, 10 aa, synthetic construct
GFSLSTSGMC

SEQ ID NO: 33 - protein, 7 aa, synthetic construct
IDWDDDK

SEQ ID NO: 34 - protein, 14 aa, synthetic construct
ARTPYSGSYNWFDP

SEQ ID NO: 35 - protein, 122 aa, synthetic construct
QVTLRESGPALVKPTQTLTLTCTFSGFSLSTSGMCVSWIRQPPGKALEWLALIDWDDDKYYSTSLKTRLTISKDTSKNQVVLTMTNMDPVDTATYYCARTPYSGSYNWFDPWGQGTLVTVSS

SEQ ID NO: 36 - DNA, 366 bp, synthetic construct
  source 1..366: mol_type = other DNA
caggtcaccttgagggagtctggtcctgcgctggtgaaacccacacagaccctcacactgacctgcaccttctctgggttctcactcagcactagtggaatgtgtgtgagctggatccgtcagcccccagggaaggccctggagtggcttgcactcattgattgggatgatgataaatactacagcacatctctgaagaccaggctcaccatctccaaggacacctccaaaaaccaggtggtccttacaatgaccaacatggaccctgtggacacagccacgtattattgtgcacggaccccctatagtgggagctacaactggttcgacccctggggccagggaaccctggtcaccgtctcctca

SEQ ID NO: 37 - protein, 8 aa, synthetic construct
GFTFSTYG

SEQ ID NO: 38 - protein, 8 aa, synthetic construct
ISYDGSNK

SEQ ID NO: 39 - protein, 10 aa, synthetic construct
ARGRVGTLDY

SEQ ID NO: 40 - protein, 11 aa, synthetic construct
RGLVYSDVNTN

SEQ ID NO: 41 - skipped

SEQ ID NO: 42 - protein, 9 aa, synthetic construct
MQGTHWPWT

SEQ ID NO: 43 - protein, 117 aa, synthetic construct
QVQLVESGGGVVQPGRSLRLSCAASGFTFSTYGMHWVRQAPGKGLEWVAVISYDGSNKYHGDAVKGRFTISRDNSKNTLYLQMNSLRAEDTAVYYCARGRVGTLDYWGQGTLVTVSS

SEQ ID NO: 44 - protein, 112 aa, synthetic construct
DVVMTQSPLSLPVSLGQPASISCRSSRGLVYSDVNTNLNWFQQRPGQSPRRLIYKVSNRDSGVPDRFSGSGSGTDFTLKISRVEAEDVGVYYCMQGTHWPWTFGQGTKVEIK

SEQ ID NO: 45 - DNA, 351 bp, synthetic construct
  source 1..351: mol_type = other DNA
caggtgcagctggtggagtctgggggaggcgtggtccagcctgggaggtccctgagactctcctgtgcagcctctggatttaccttcagtacctatggcatgcactgggtccgccaggctccaggcaaggggctggagtgggtggcagtcatatcatatgatggaagtaataaatatcatggagacgccgtgaagggccggttcaccatctccagagacaattccaagaacacgctgtatctgcaaatgaacagcctgagagctgaggacacggctgtgtattactgtgcgagggggagagtgggaactcttgactattggggccagggaaccctggtcaccgtctcctca

SEQ ID NO: 46 - DNA, 336 bp, synthetic construct
  source 1..336: mol_type = other DNA
gatgttgtgatgactcagtctccactctccctgcccgtcagccttggacagccggcctccatctcctgcaggtctagtcgaggcctcgtatacagtgatgtaaacaccaacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccttggacgttcggccaagggaccaaggtggaaatcaaa

SEQ ID NO: 47 - protein, 8 aa, synthetic construct
GFTFNSYG

SEQ ID NO: 48 - protein, 16 aa, synthetic construct
ARDNYDSSGYNWYFDL

SEQ ID NO: 49 - protein, 6 aa, synthetic construct
SLRSYY

SEQ ID NO: 50 - skipped

SEQ ID NO: 51 - protein, 11 aa, synthetic construct
NSRDSSGNLWV

SEQ ID NO: 52 - protein, 123 aa, synthetic construct
QVQLVESGGGVVQPGRSLRLSCAASGFTFNSYGMHWVRQAPGKGLEWVAIISYDGSNKYYADSVKGRFTISRDNSKNTLYLQMNSLRAEDTAVYYCARDNYDSSGYNWYFDLWGRGTLVTVSS

SEQ ID NO: 53 - protein, 108 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSRDSSGNLWVFGGGTKLTVL

SEQ ID NO: 54 - DNA, 369 bp, synthetic construct
  source 1..369: mol_type = other DNA
caggtgcagctggtggagtctgggggaggcgtggtccagcctgggaggtccctgagactctcctgtgcagcctctggattcaccttcaacagctatggcatgcactgggtccgccaggctccaggcaaggggctggagtgggtggcaattatatcatatgatggaagtaataaatactatgcagactccgtgaagggccgattcaccatctccagagacaattccaagaacacgctgtatctgcaaatgaacagcctgagagctgaggacacggctgtgtattattgtgcgagagataattatgatagtagtggttataactggtacttcgatctctggggccgtggcaccctggtcactgtctcctca

SEQ ID NO: 55 - DNA, 324 bp, synthetic construct
  source 1..324: mol_type = other DNA
tcttctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaacctctgggtgttcggcggagggaccaagctgaccgtccta

SEQ ID NO: 56 - protein, 8 aa, synthetic construct
GDSISSYY

SEQ ID NO: 57 - protein, 7 aa, synthetic construct
IYTSGNT

SEQ ID NO: 58 - protein, 14 aa, synthetic construct
ARDGDNRDSDAFDI

SEQ ID NO: 59 - protein, 7 aa, synthetic construct
QNISSSY

SEQ ID NO: 60 - skipped

SEQ ID NO: 61 - protein, 9 aa, synthetic construct
QQYGSSPLT

SEQ ID NO: 62 - protein, 120 aa, synthetic construct
QVQLQESGPGLVKPSETLSLTCTVSGDSISSYYWSWIRQPAGKGLEWIGRIYTSGNTNYNPSLKSRVTMSVDKSKNQFSLKLRSVTAADTAVYYCARDGDNRDSDAFDIWGQGTMVTVSS

SEQ ID NO: 63 - protein, 102 aa, synthetic construct
SPGTLSLSPGERATLSCRASQNISSSYLAWCQQKPGQAPRLFIYGTSRRATGIPDRFSGSGSGTDFTLTISRLEPEDFAVYYCQQYGSSPLTFGGGTKVEIK

SEQ ID NO: 64 - DNA, 360 bp, synthetic construct
  source 1..360: mol_type = other DNA
caggtgcagctgcaggagtcgggcccaggactggtgaagccttcggagaccctgtccctcacctgcactgtctctggtgactccatcagtagttattactggagctggatccggcagcccgccgggaagggactggagtggattggacgtatctataccagtgggaacaccaactacaacccctccctcaagagtcgagtcaccatgtcagtagacaagtccaagaaccagttctccctgaagctgaggtctgtgaccgccgcggacacggccgtgtattactgtgcgagagatggtgataaccgggactctgatgcttttgatatctggggccaagggacaatggtcaccgtctcttca

SEQ ID NO: 65 - DNA, 324 bp, synthetic construct
  source 1..324: mol_type = other DNA
gaaattgtgttgacgcagtctccaggcaccctgtctttgtctccaggggaaagagccaccctctcctgcagggccagtcagaatattagcagcagctacttagcctggtgccagcagaaacctggccaggctcccaggctcttcatctatggtacatcccgcagggccactggcatcccagacaggttcagtggcagtgggtctgggacagacttcactctcaccatcagcagactggagcctgaagattttgcagtgtattactgtcagcagtatggtagttcacctctcactttcggcggagggaccaaggtggagatcaaa

SEQ ID NO: 66 - protein, 8 aa, synthetic construct
GFSFTSHW

SEQ ID NO: 67 - protein, 8 aa, synthetic construct
IYPGDSDT

SEQ ID NO: 68 - protein, 15 aa, synthetic construct
ARHETGNGYSYGMDV

SEQ ID NO: 69 - protein, 11 aa, synthetic construct
QSLLHSNGYNY

SEQ ID NO: 70 - skipped

SEQ ID NO: 71 - protein, 9 aa, synthetic construct
MQALQPPLT

SEQ ID NO: 72 - protein, 122 aa, synthetic construct
EVQLVQSGADVKKPGESLKISCKGSGFSFTSHWIAWVRQMPGKGLEWMGIIYPGDSDTRYSPSLQGRVTISADKSISTAYLQWSSLKASDTAMYFCARHETGNGYSYGMDVWGQGTTVTVSS

SEQ ID NO: 73 - protein, 112 aa, synthetic construct
DIVMTQSPLSLPVTPGEPASISCRSSQSLLHSNGYNYLDWYLQKPGQSPQLLFSLGSNRASGVPDRFSGSGSGTDFSLKISRVEAEDVGLYYCMQALQPPLTFGGGTKVEIK

SEQ ID NO: 74 - DNA, 366 bp, synthetic construct
  source 1..366: mol_type = other DNA
gaggtgcagctggtgcagtctggagcagacgtgaaaaagcccggggagtctctgaagatctcctgtaagggttctggattcagttttaccagccactggatcgcctgggtgcgccagatgcccgggaaaggcctggagtggatggggataatctatcctggtgactctgataccagatacagcccgtccttacaaggccgggtcaccatctcagccgacaagtccatcagcaccgcctacctgcagtggagcagcctgaaggcctcggacaccgccatgtatttctgtgcgaggcatgaaactgggaatggctactcctacggaatggacgtctggggccaagggaccacggtcaccgtctcctca

SEQ ID NO: 75 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgttctctttgggttctaatcgggcctccggggtccctgacaggttcagtggcagtggatcaggcacagatttttcactgaaaatcagcagagtggaggctgaggatgttgggctttattactgcatgcaagctctacaacctccgctcactttcggcggagggaccaaggtggagatcaaa

SEQ ID NO: 76 - protein, 8 aa, synthetic construct
GGSISTYY

SEQ ID NO: 77 - protein, 14 aa, synthetic construct
ARDGDNRNSDAFDI

SEQ ID NO: 78 - protein, 9 aa, synthetic construct
SSNIGAGYD

SEQ ID NO: 79 - skipped

SEQ ID NO: 80 - protein, 11 aa, synthetic construct
QSYDRSLSGWV

SEQ ID NO: 81 - protein, 120 aa, synthetic construct
QVQLQESGPGLVKPSETLSLTCTVSGGSISTYYWSWIRQPAGKGLEWIGRIYTSGNTNYNPSLKSRVTMSVDTSKNQLSLKLRSVTAADTAVYYCARDGDNRNSDAFDIWGQGTMVTVSS

SEQ ID NO: 82 - protein, 111 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDRSLSGWVFGGGTKLTVL

SEQ ID NO: 83 - DNA, 360 bp, synthetic construct
  source 1..360: mol_type = other DNA
caggtgcagctgcaggagtcgggcccaggactggtgaagccttcggagaccctgtccctcacctgcactgtctctggtggctccatcagtacttattactggagctggatccggcagcccgccgggaagggactggagtggattgggcgtatctataccagtgggaacaccaactacaacccctccctcaagagtcgagtcaccatgtcagtagacacgtccaagaatcagctctccctgaagctgaggtctgtgaccgccgcggacacggccgtgtattactgtgcgagagatggggataaccggaactctgatgcttttgatatttggggccaagggacaatggtcaccgtctcttca

SEQ ID NO: 84 - DNA, 333 bp, synthetic construct
  source 1..333: mol_type = other DNA
cagtctgtgctgacgcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgaccgcagcctgagtggttgggtgttcggcggagggaccaagctgaccgtccta

SEQ ID NO: 85 - protein, 8 aa, synthetic construct
GFTFSAYP

SEQ ID NO: 86 - protein, 8 aa, synthetic construct
ISYDGSNN

SEQ ID NO: 87 - protein, 10 aa, synthetic construct
ARGKVGTLDF

SEQ ID NO: 88 - protein, 117 aa, synthetic construct
QIQLVESGGGVVQPGRSLRLSCAASGFTFSAYPMHWVRQAPGKGLEWVAIISYDGSNNYYADSVKGRFTISRDNSKNTMYLQINSLRAEDTGVYYCARGKVGTLDFWGQGTLVTVSS

SEQ ID NO: 89 - protein, 112 aa, synthetic construct
DVVLTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPWTFGQGTKVEIK

SEQ ID NO: 90 - DNA, 351 bp, synthetic construct
  source 1..351: mol_type = other DNA
cagatacagctggtggagtctgggggaggcgtggtccagcctgggaggtccctgagactctcctgtgcagcctctggattcaccttcagtgcctatcccatgcactgggtccgccaggctccaggcaaggggctggagtgggtggcaattatatcatatgatggaagtaataactactatgcagactccgtgaagggccgattcaccatctccagagacaattccaagaacacgatgtatctgcaaattaacagcctgagagctgaggacacgggtgtgtattactgtgcgcgagggaaagtgggaacccttgacttctggggccagggaaccctggtcaccgtctcctca

SEQ ID NO: 91 - DNA, 336 bp, synthetic construct
  source 1..336: mol_type = other DNA
gatgttgtgctgactcagtctccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccgtggacgttcggccaagggaccaaggtggaaatcaaa

SEQ ID NO: 92 - protein, 112 aa, synthetic construct
DIVMTQSPLSSPVTLGQPASISCRSSQSLVYSDGNTYLSWLQQRPGQPPRLLIYKISNRFSGVPDRFSGSGAGTDFTLKISRVEAEDVGVYYCMQSTQFPHTFGQGTKLEIK

SEQ ID NO: 93 - DNA, 363 bp, synthetic construct
  source 1..363: mol_type = other DNA
gaggtgcagctgttggagtctgggggaggcttggtccagcctggggggtccctgagactctcctgtgcagcctctggattcacctttagcacctatgccatgagctgggtccgccaggctccagggaaggggctggagtgggtctcagctattagtggtcgtggtggtagcacatactacacagactccgtgaagggccggttcaccatctccagagacaattccaagaatacggtgtctctgcaaatgaacagcctgagagccgaggacacggccgtatattactgtgcgggccggggagattactactactactacggtatggacgtctggggccaagggaccacggtcaccgtctccgca

SEQ ID NO: 94 - DNA, 336 bp, synthetic construct
  source 1..336: mol_type = other DNA
gatattgtgatgacccagagtccactctcctcacctgtcacccttggacagccggcctccatctcctgcaggtctagtcaaagcctcgtatacagtgatggaaacacctacttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcatgcaatctacacaatttcctcacacttttggccaggggaccaagctggagatcaaa

SEQ ID NO: 95 - protein, 330 aa, synthetic construct [sequence omitted]

SEQ ID NO: 96 - protein, 107 aa, synthetic construct [sequence omitted]

SEQ ID NO: 97 - protein, 451 aa, synthetic construct
EVQLLESGGGLVQPGGSLRLSCAASGFTFSTYAMSWVRQAPGKGLEWVSAISGRGGSTYYTDSVKGRFTISRDNSKNTVSLQMNSLRAEDTAVYYCAGRGDYYYYYGMDVWGQGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 219 aa, synthetic construct
DIVMTQSPLSSPVTLGQPASFSCRSSQSLVYSDGNTYLSWLQQRPGQPPRLLIYKISNRFSGVPDRFSGSGAGTDFTLKISRVEAEDVGVYYCMQSTQFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 106 aa, synthetic construct
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 100 - protein, 451 aa, synthetic construct
EVHLLESGGGLVQPGGSLRLSCAASGFIFSSNAMSWVRQAPGKGLEWVSAISGYGGNTYYADSVKGRFTISRDNSKNTLYLQMNSLRAEDTAVYYCAKWGTYIVGATGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 216 aa, synthetic construct
QSALTQPPSASGSPGQSVTISCTGTSNDVGGYNYVSWYQQHPGKAPKLLIYEVSKRPSGVPDRFSGSQSGNTASLTVSGLQAEDEADYYCSSYAGSNNWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 102 - protein, 450 aa, synthetic construct
QVQLQQSGPGLVKPSQTLSLTCAISGDSVSSNSAAWNWIRQSPSRGLEWLGRTYFRSKWYNVYAVSVKSRITINPDTSKNQFSLQLNSVTPEDTAVYYCASEGGSYYDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 214 aa, synthetic construct
DIQMTQSPSSVSASVGDRVTITCRASQGISNWLTWYQQKPGKAPKLLIYAASSLQSGVPSRFSGSGSGTDFTLTISSLQPEDFATYYCQQADSFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 19 aa, synthetic construct
GGGGSGGGGSGGGSGGGGS

SEQ ID NO: 105 - protein, 15 aa, synthetic construct
GGGGSGGGGSGGGGS

SEQ ID NO: 106 - protein, 24 aa, synthetic construct
GGGGSGGGGSGGGGSGGGSGGGGS

SEQ ID NO: 107 - protein, 29 aa, synthetic construct
GGGGSGGGGSGGGGSGGGGSGGGSGGGGS

SEQ ID NO: 108 - protein, 5 aa, synthetic construct
GGGGS

SEQ ID NO: 109 - protein, 10 aa, synthetic construct
GGGGSGGGGS